SEQUENCE LISTING (156 sequences)

SEQ ID NO: 1 - protein, 106 aa, Mus musculus
DIVLSQSPAILSASPGEKVTMTCRASSSVNYMHWYQQKPGSSPKPWISATSNLASGVPARFSGSGSGTSYSLTISRVEAEDAATYYCQQWSSNPPTFGGGTMLEIK

SEQ ID NO: 2 - protein, 123 aa, Mus musculus
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGASGIDYAMDYWGQGTSLTVSS

SEQ ID NO: 3 - protein, 10 aa, Mus musculus
RASSSVNYMH

SEQ ID NO: 4 - protein, 7 aa, Mus musculus
ATSNLAS

SEQ ID NO: 5 - protein, 9 aa, Mus musculus
QQWSSNPPT

SEQ ID NO: 6 - protein, 10 aa, Mus musculus
GFTFNNYAMN

SEQ ID NO: 7 - protein, 19 aa, Mus musculus
RIRSKSNNYATFYADSVKD

SEQ ID NO: 8 - protein, 12 aa, Mus musculus
RGASGIDYAMDY

SEQ ID NO: 9 - protein, 10 aa, Mus musculus
RASSSVSYMH

SEQ ID NO: 10 - protein, 9 aa, Mus musculus
QQWSSNPPT

SEQ ID NO: 11 - protein, 10 aa, Mus musculus
GFTFNTYAMN

SEQ ID NO: 12 - protein, 19 aa, Mus musculus
RIRSKSNNYATYYADSVKD

SEQ ID NO: 13 - protein, 12 aa, Mus musculus
RGGKETDYAMDY

SEQ ID NO: 14 - protein, 10 aa, Artificial Sequence
  Synthetic peptide
RASSSVRYMH

SEQ ID NO: 15 - protein, 9 aa, Artificial Sequence
  Synthetic peptide
QQWSYNPPT

SEQ ID NO: 16 - protein, 9 aa, Artificial Sequence
  Synthetic peptide
QQWSKNPPT

SEQ ID NO: 17 - protein, 9 aa, Artificial Sequence
  Synthetic peptide
QQWRKNPPT

SEQ ID NO: 18 - protein, 9 aa, Artificial Sequence
  Synthetic peptide
QQWRSNPPT

SEQ ID NO: 19 - protein, 12 aa, Artificial Sequence
  Synthetic peptide
RGARGIHYAMDY

SEQ ID NO: 20 - protein, 10 aa, Artificial Sequence
  Synthetic peptide
GFTFNKYAMN

SEQ ID NO: 21 - protein, 19 aa, Artificial Sequence
  Synthetic peptide
RIRSKKNNYATFYADSVKD

SEQ ID NO: 22 - protein, 12 aa, Artificial Sequence
  Synthetic peptide
RGARGIDYAMDY

SEQ ID NO: 23 - protein, 12 aa, Artificial Sequence
  Synthetic peptide
RGASGIHYAMDY

SEQ ID NO: 24 - protein, 19 aa, Artificial Sequence
  Synthetic peptide
RIRSKSNRYATFYADSVKD

SEQ ID NO: 25 - protein, 12 aa, Artificial Sequence
  Synthetic peptide
RGAKGIDYAMDY

SEQ ID NO: 26 - protein, 12 aa, Artificial Sequence
  Synthetic peptide
RGASSIDYAMDY

SEQ ID NO: 27 - protein, 19 aa, Artificial Sequence
  Synthetic peptide
RIRSKRNNYATFYADSVKD

SEQ ID NO: 28 - protein, 12 aa, Artificial Sequence
  Synthetic peptide
RGASGINYAMDY

SEQ ID NO: 29 - protein, 12 aa, Artificial Sequence
  Synthetic peptide
RGASKIDYAMDY

SEQ ID NO: 30 - protein, 12 aa, Artificial Sequence
  Synthetic peptide
RGASGIAYAMDY

SEQ ID NO: 31 - protein, 12 aa, Artificial Sequence
  Synthetic peptide
RGASGIRYAMDY

SEQ ID NO: 32 - protein, 12 aa, Artificial Sequence
  Synthetic peptide
RGASGIDYAMDK

SEQ ID NO: 33 - protein, 213 aa, Mus musculus
DIVLSQSPAILSASPGEKVTMTCRASSSVNYMHWYQQKPGSSPKPWISATSNLASGVPARFSGSGSGTSYSLTISRVEAEDAATYYCQQWSSNPPTFGGGTMLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 455 aa, Mus musculus
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGASGIDYAMDYWGQGTSLTVSSEFASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 369 bp, Mus musculus
gaagtgatgctggtggagtctggtggaggattggtgcagcctaaagggtcattgaaactctcatgtgcagcctctggattcaccttcaataactacgccatgaattgggtccgccaggctccaggaaagggtttggaatgggttgctcgcataagaagtaaaagtaataattatgcaacattttatgccgattcagtgaaagacaggttcaccatctccagagatgattcacaaagcatgctctatctgcaaatgaacaacttgaaaactgaggacacagccatgtattactgtgtgagacggggggcttcagggattgactatgctatggactactggggtcaaggaacctcactcaccgtctcctca

SEQ ID NO: 36 - DNA, 318 bp, Mus musculus
gatatcgttctctcccagtctccagcaatcctgtctgcatctccaggggaaaaggtcacaatgacttgcagggccagctcaagtgtaaattacatgcactggtaccagcagaagccaggatcctcccccaaaccctggatttctgccacatccaacctggcttctggagtccctgctcgcttcagtggcagtgggtctgggacctcttactctctcacaatcagcagagtggaggctgaagatgctgccacttattactgccagcagtggagtagtaacccacccacgttcggaggggggaccatgctggaaataaaa

SEQ ID NO: 37 - protein, 23 aa, Mus musculus
DIVLSQSPAILSASPGEKVTMTC

SEQ ID NO: 38 - protein, 15 aa, Mus musculus
WYQQKPGSSPKPWIS

SEQ ID NO: 39 - protein, 32 aa, Mus musculus
GVPARFSGSGSGTSYSLTISRVEAEDAATYYC

SEQ ID NO: 40 - protein, 10 aa, Mus musculus
FGGGTMLEIK

SEQ ID NO: 41 - protein, 25 aa, Mus musculus
EVMLVESGGGLVQPKGSLKLSCAAS

SEQ ID NO: 42 - protein, 14 aa, Mus musculus
WVRQAPGKGLEWVA

SEQ ID NO: 43 - protein, 32 aa, Mus musculus
RFTISRDDSQSMLYLQMNNLKTEDTAMYYCVR

SEQ ID NO: 44 - protein, 11 aa, Mus musculus
WGQGTSLTVSS

SEQ ID NO: 45 - protein, 106 aa, Mus musculus
DIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGTKLEIK

SEQ ID NO: 46 - protein, 123 aa, Mus musculus
EVMLVESGEGLVQPKGSLKLSCAASGFTFNTYAMNWVRQAPGKGLEWVARIRSKSNNYATYYADSVKDRFTISRDDSQSMLYLQMNNLKTEDTAMYYCVRRGGKETDYAMDYWGQGTSVTVSS

SEQ ID NO: 47 - protein, 106 aa, Artificial Sequence
  Synthetic peptide
DIVLSQSPAILSASPGEKVTMTCRASSSVRYMHWYQQKPGSSPKPWISATSNLASGVPARFSGSGSGTSYSLTISRVEAEDAATYYCQQWSYNPPTFGGGTMLEIK

SEQ ID NO: 48 - protein, 123 aa, Artificial Sequence
  Synthetic peptide
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGARGIHYAMDYWGQGTSLTVSS

SEQ ID NO: 49 - protein, 106 aa, Artificial Sequence; identical to SEQ ID NO: 47
  Synthetic peptide

SEQ ID NO: 50 - protein, 123 aa, Artificial Sequence
  Synthetic peptide
EVMLVESGGGLVQPKGSLKLSCAASGFTFNKYAMNWVRQAPGKGLEWVARIRSKKNNYATFYADSVKDRFTISRDDSQSMLYLQMNNLKTEDTAMYYCVRRGASGIDYAMDYWGQGTSLTVSS

SEQ ID NO: 51 - protein, 106 aa, Artificial Sequence; identical to SEQ ID NO: 47
  Synthetic peptide

SEQ ID NO: 52 - protein, 123 aa, Artificial Sequence
  Synthetic peptide
EVMLVESGGGLVQPKGSLKLSCAASGFTFNNYAMNWVRQAPGKGLEWVARIRSKKNNYATFYADSVKDRFTISRDDSQSMLYLQMNNLKTEDTAMYYCVRRGARGIDYAMDYWGQGTSLTVSS

SEQ ID NO: 53 - protein, 106 aa, Artificial Sequence; identical to SEQ ID NO: 47
  Synthetic peptide

SEQ ID NO: 54 - protein, 123 aa, Artificial Sequence
  Synthetic peptide
EVMLVESGGGLVQPKGSLKLSCAASGFTFNKYAMNWVRQAPGKGLEWVARIRSKKNNYATFYADSVKDRFTISRDDSQSMLYLQMNNLKTEDTAMYYCVRRGASGIHYAMDYWGQGTSLTVSS

SEQ ID NO: 55 - protein, 106 aa, Artificial Sequence; identical to SEQ ID NO: 47
  Synthetic peptide

SEQ ID NO: 56 - protein, 123 aa, Artificial Sequence
  Synthetic peptide
EVMLVESGGGLVQPKGSLKLSCAASGFTFNNYAMNWVRQAPGKGLEWVARIRSKKNNYATFYADSVKDRFTISRDDSQSMLYLQMNNLKTEDTAMYYCVRRGARGIHYAMDYWGQGTSLTVSS

SEQ ID NO: 57 - protein, 106 aa, Artificial Sequence; identical to SEQ ID NO: 47
  Synthetic peptide

SEQ ID NO: 58 - protein, 123 aa, Artificial Sequence
  Synthetic peptide
EVMLVESGGGLVQPKGSLKLSCAASGFTFNKYAMNWVRQAPGKGLEWVARIRSKKNNYATFYADSVKDRFTISRDDSQSMLYLQMNNLKTEDTAMYYCVRRGARGIDYAMDYWGQGTSLTVSS

SEQ ID NO: 59 - protein, 106 aa, Artificial Sequence
  Synthetic peptide
DIVLSQSPAILSASPGEKVTMTCRASSSVRYMHWYQQKPGSSPKPWISATSNLASGVPARFSGSGSGTSYSLTISRVEAEDAATYYCQQWSKNPPTFGGGTMLEIK

SEQ ID NO: 60 - protein, 123 aa, Artificial Sequence
  Synthetic peptide
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGARGIDYAMDYWGQGTSLTVSS

SEQ ID NO: 61 - protein, 106 aa, Artificial Sequence; identical to SEQ ID NO: 59
  Synthetic peptide

SEQ ID NO: 62 - protein, 123 aa, Artificial Sequence; identical to SEQ ID NO: 48
  Synthetic peptide

SEQ ID NO: 63 - protein, 106 aa, Artificial Sequence; identical to SEQ ID NO: 59
  Synthetic peptide

SEQ ID NO: 64 - protein, 123 aa, Artificial Sequence
  Synthetic peptide
EVMLVESGGGLVQPKGSLKLSCAASGFTFNNYAMNWVRQAPGKGLEWVARIRSKKNNYATFYADSVKDRFTISRDDSQSMLYLQMNNLKTEDTAMYYCVRRGASGIHYAMDYWGQGTSLTVSS

SEQ ID NO: 65 - protein, 106 aa, Artificial Sequence; identical to SEQ ID NO: 59
  Synthetic peptide

SEQ ID NO: 66 - protein, 123 aa, Artificial Sequence
  Synthetic peptide
EVMLVESGGGLVQPKGSLKLSCAASGFTFNKYAMNWVRQAPGKGLEWVARIRSKSNNYATFYADSVKDRFTISRDDSQSMLYLQMNNLKTEDTAMYYCVRRGARGIHYAMDYWGQGTSLTVSS

SEQ ID NO: 67 - protein, 106 aa, Artificial Sequence
  Synthetic peptide
DIVLSQSPAILSASPGEKVTMTCRASSSVRYMHWYQQKPGSSPKPWISATSNLASGVPARFSGSGSGTSYSLTISRVEAEDAATYYCQQWRKNPPTFGGGTMLEIK

SEQ ID NO: 68 - protein, 123 aa, Artificial Sequence
  Synthetic peptide
EVMLVESGGGLVQPKGSLKLSCAASGFTFNNYAMNWVRQAPGKGLEWVARIRSKKNNYATFYADSVKDRFTISRDDSQSMLYLQMNNLKTEDTAMYYCVRRGASGIDYAMDYWGQGTSLTVSS

SEQ ID NO: 69 - protein, 106 aa, Artificial Sequence
  Synthetic peptide
DIVLSQSPAILSASPGEKVTMTCRASSSVNYMHWYQQKPGSSPKPWISATSNLASGVPARFSGSGSGTSYSLTISRVEAEDAATYYCQQWRKNPPTFGGGTMLEIK

SEQ ID NO: 70 - protein, 123 aa, Artificial Sequence; identical to SEQ ID NO: 64
  Synthetic peptide

SEQ ID NO: 71 - protein, 106 aa, Artificial Sequence
  Synthetic peptide
DIVLSQSPAILSASPGEKVTMTCRASSSVNYMHWYQQKPGSSPKPWISATSNLASGVPARFSGSGSGTSYSLTISRVEAEDAATYYCQQWRSNPPTFGGGTMLEIK

SEQ ID NO: 72 - protein, 123 aa, Artificial Sequence
  Synthetic peptide
EVMLVESGGGLVQPKGSLKLSCAASGFTFNNYAMNWVRQAPGKGLEWVARIRSKSNRYATFYADSVKDRFTISRDDSQSMLYLQMNNLKTEDTAMYYCVRRGARGIDYAMDYWGQGTSLTVSS

SEQ ID NO: 73 - protein, 106 aa, Artificial Sequence; identical to SEQ ID NO: 1
  Synthetic peptide

SEQ ID NO: 74 - protein, 123 aa, Artificial Sequence
  Synthetic peptide
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGAKGIDYAMDYWGQGTSLTVSS

SEQ ID NO: 75 - protein, 106 aa, Artificial Sequence; identical to SEQ ID NO: 1
  Synthetic peptide

SEQ ID NO: 76 - protein, 123 aa, Artificial Sequence
  Synthetic peptide
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGASSIDYAMDYWGQGTSLTVSS

SEQ ID NO: 77 - protein, 106 aa, Artificial Sequence; identical to SEQ ID NO: 47
  Synthetic peptide

SEQ ID NO: 78 - protein, 123 aa, Artificial Sequence
  Synthetic peptide
EVMLVESGGGLVQPKGSLKLSCAASGFTFNNYAMNWVRQAPGKGLEWVARIRSKRNNYATFYADSVKDRFTISRDDSQSMLYLQMNNLKTEDTAMYYCVRRGASGIHYAMDYWGQGTSLTVSS

SEQ ID NO: 79 - protein, 106 aa, Artificial Sequence
  Synthetic peptide
DIVLSQSPAILSASPGEKVTMTCRASSSVRYMHWYQQKPGSSPKPWISATSNLASGVPARFSGSGSGTSYSLTISRVEAEDAATYYCQQWSSNPPTFGGGTMLEIK

SEQ ID NO: 80 - protein, 123 aa, Artificial Sequence
  Synthetic peptide
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGASGINYAMDYWGQGTSLTVSS

SEQ ID NO: 81 - protein, 106 aa, Artificial Sequence; identical to SEQ ID NO: 1
  Synthetic peptide

SEQ ID NO: 82 - protein, 123 aa, Artificial Sequence
  Synthetic peptide
EVMLVESGGGLVQPKGSLKLSCAASGFTFNKYAMNWVRQAPGKGLEWVARIRSKSNNYATFYADSVKDRFTISRDDSQSMLYLQMNNLKTEDTAMYYCVRRGASKIDYAMDYWGQGTSLTVSS

SEQ ID NO: 83 - protein, 106 aa, Artificial Sequence; identical to SEQ ID NO: 1
  Synthetic peptide

SEQ ID NO: 84 - protein, 123 aa, Artificial Sequence
  Synthetic peptide
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGASGIAYAMDYWGQGTSLTVSS

SEQ ID NO: 85 - protein, 106 aa, Artificial Sequence; identical to SEQ ID NO: 1
  Synthetic peptide

SEQ ID NO: 86 - protein, 123 aa, Artificial Sequence
  Synthetic peptide
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGASGIRYAMDYWGQGTSLTVSS

SEQ ID NO: 87 - protein, 106 aa, Artificial Sequence; identical to SEQ ID NO: 1
  Synthetic peptide

SEQ ID NO: 88 - protein, 123 aa, Artificial Sequence
  Synthetic peptide
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGASGIDYAMDKWGQGTSLTVSS

SEQ ID NO: 89 - protein, 10 aa, Artificial Sequence
  Synthetic peptide
RASRSVNYMH

SEQ ID NO: 90 - protein, 10 aa, Artificial Sequence
  Synthetic peptide
RASSSVKYMH

SEQ ID NO: 91 - protein, 7 aa, Artificial Sequence
  Synthetic peptide
ATLNLAS

SEQ ID NO: 92 - protein, 7 aa, Artificial Sequence
  Synthetic peptide
ATINLAS

SEQ ID NO: 93 - protein, 9 aa, Artificial Sequence
  Synthetic peptide
QQWRSNPPT

SEQ ID NO: 94 - protein, 9 aa, Artificial Sequence
  Synthetic peptide
QQWSRNPPT

SEQ ID NO: 95 - protein, 10 aa, Artificial Sequence
  Synthetic peptide
GFTFNRYAMN

SEQ ID NO: 96 - protein, 19 aa, Artificial Sequence
  Synthetic peptide
RIRSKSNKYATFYADSVKD

SEQ ID NO: 97 - protein, 19 aa, Artificial Sequence
  Synthetic peptide
RIRSKSNNYATFYAPSVKD

SEQ ID NO: 98 - protein, 12 aa, Artificial Sequence
  Synthetic peptide
RGASGIHYAMDY

SEQ ID NO: 99 - protein, 12 aa, Artificial Sequence
  Synthetic peptide
RGASGINYAMDY

SEQ ID NO: 100 - protein, 23 aa, Mus musculus
DIVLSQSPAILSASPGEKVTMTC

SEQ ID NO: 101 - protein, 15 aa, Mus musculus
WYQQKPGSSPKPWIY

SEQ ID NO: 102 - protein, 32 aa, Mus musculus; identical to SEQ ID NO: 39

SEQ ID NO: 103 - protein, 10 aa, Mus musculus
FGGGTKLEIK

SEQ ID NO: 104 - protein, 25 aa, Mus musculus
EVMLVESGEGLVQPKGSLKLSCAAS

SEQ ID NO: 105 - protein, 14 aa, Mus musculus
WVRQAPGKGLEWVA

SEQ ID NO: 106 - protein, 32 aa, Mus musculus; identical to SEQ ID NO: 43

SEQ ID NO: 107 - protein, 11 aa, Mus musculus
WGQGTSVTVSS

SEQ ID NO: 108 - DNA, 318 bp, Artificial Sequence
  Synthetic DNA
gatatcgttctctcccagtctccagcaatcctgtctgcatctccaggggaaaaggtcacaatgacttgcagggccagctcaagtgtacgctacatgcactggtaccagcagaagccaggatcctcccccaaaccctggatttctgccacatccaacctggcttctggagtccctgctcgcttcagtggcagtgggtctgggacctcttactctctcacaatcagcagagtggaggctgaagatgctgccacttattactgccagcagtggagttataacccacccacgttcggaggggggaccatgctggaaataaaa

SEQ ID NO: 109 - DNA, 369 bp, Artificial Sequence
  Synthetic DNA
gaagtgatgctggtggagtctggtggaggattggtgcagcctaaagggtcattgaaactctcatgtgcagcctctggattcaccttcaataagtacgccatgaattgggtccgccaggctccaggaaagggtttggaatgggttgctcgcataagaagtaaaaagaataattatgcaacattttatgccgattcagtgaaagacaggttcaccatctccagagatgattcacaaagcatgctctatctgcaaatgaacaacttgaaaactgaggacacagccatgtattactgtgtgagacggggggctcgtgggattgactatgctatggactactggggtcaaggaacctcactcaccgtctcctca

SEQ ID NO: 110 - DNA, 318 bp, Artificial Sequence
  Synthetic DNA
gatatcgttctctcccagtctccagcaatcctgtctgcatctccaggggaaaaggtcacaatgacttgcagggccagctcaagtgtacgctacatgcactggtaccagcagaagccaggatcctcccccaaaccctggatttctgccacatccaacctggcttctggagtccctgctcgcttcagtggcagtgggtctgggacctcttactctctcacaatcagcagagtggaggctgaagatgctgccacttattactgccagcagtggagtaagaacccacccacgttcggaggggggaccatgctggaaataaaa

SEQ ID NO: 111 - DNA, 369 bp, Artificial Sequence
  Synthetic DNA
gaagtgatgctggtggagtctggtggaggattggtgcagcctaaagggtcattgaaactctcatgtgcagcctctggattcaccttcaataagtacgccatgaattgggtccgccaggctccaggaaagggtttggaatgggttgctcgcataagaagtaaaagtaataattatgcaacattttatgccgattcagtgaaagacaggttcaccatctccagagatgattcacaaagcatgctctatctgcaaatgaacaacttgaaaactgaggacacagccatgtattactgtgtgagacggggggctcgtgggattcactatgctatggactactggggtcaaggaacctcactcaccgtctcctca

SEQ ID NO: 112 - DNA, 318 bp, Artificial Sequence; identical to SEQ ID NO: 110
  Synthetic DNA

SEQ ID NO: 113 - DNA, 369 bp, Artificial Sequence
  Synthetic DNA
gaagtgatgctggtggagtctggtggaggattggtgcagcctaaagggtcattgaaactctcatgtgcagcctctggattcaccttcaataactacgccatgaattgggtccgccaggctccaggaaagggtttggaatgggttgctcgcataagaagtaaaaagaataattatgcaacattttatgccgattcagtgaaagacaggttcaccatctccagagatgattcacaaagcatgctctatctgcaaatgaacaacttgaaaactgaggacacagccatgtattactgtgtgagacggggggcttcagggattcactatgctatggactactggggtcaaggaacctcactcaccgtctcctca

SEQ ID NO: 114 - DNA, 318 bp, Artificial Sequence; identical to SEQ ID NO: 108
  Synthetic DNA

SEQ ID NO: 115 - DNA, 369 bp, Artificial Sequence
  Synthetic DNA
gaagtgatgctggtggagtctggtggaggattggtgcagcctaaagggtcattgaaactctcatgtgcagcctctggattcaccttcaataagtacgccatgaattgggtccgccaggctccaggaaagggtttggaatgggttgctcgcataagaagtaaaaagaataattatgcaacattttatgccgattcagtgaaagacaggttcaccatctccagagatgattcacaaagcatgctctatctgcaaatgaacaacttgaaaactgaggacacagccatgtattactgtgtgagacggggggcttcagggattgactatgctatggactactggggtcaaggaacctcactcaccgtctcctca

SEQ ID NO: 116 - protein, 10 aa, Artificial Sequence
  Synthetic peptide
RASSSVXYMH

SEQ ID NO: 117 - protein, 7 aa, Artificial Sequence
  Synthetic peptide
ATSNLAS

SEQ ID NO: 118 - protein, 9 aa, Artificial Sequence
  Synthetic peptide
QQWXXNPPT

SEQ ID NO: 119 - protein, 5 aa, Artificial Sequence
  Synthetic peptide
XYAMN

SEQ ID NO: 120 - protein, 19 aa, Artificial Sequence
  Synthetic peptide
RIRSKXNXYATXYADSVKD

SEQ ID NO: 121 - protein, 12 aa, Artificial Sequence
  Synthetic peptide
RGXXXXXYAMDX

SEQ ID NO: 122 - protein, 10 aa, Artificial Sequence
  Synthetic peptide
GFTFNXYAMN

SEQ ID NO: 123 - protein, 19 aa, Artificial Sequence
  Synthetic peptide
RIRSKXNXYATXYADSVKD

SEQ ID NO: 124 - protein, 12 aa, Artificial Sequence
  Synthetic peptide
RGXXXXXYAMDX

SEQ ID NO: 125 - protein, 10 aa, Artificial Sequence
  Synthetic peptide
RCPEAVINLP

SEQ ID NO: 126 - protein, 10 aa, Artificial Sequence
  Synthetic peptide
RGSVLVTTKH

SEQ ID NO: 127 - protein, 10 aa, Artificial Sequence
  Synthetic peptide
RLSASVNMMH

SEQ ID NO: 128 - protein, 7 aa, Artificial Sequence
  Synthetic peptide
PKTRTTP

SEQ ID NO: 129 - protein, 7 aa, Artificial Sequence
  Synthetic peptide
TSDPAAS

SEQ ID NO: 130 - protein, 7 aa, Artificial Sequence
  Synthetic peptide
AASGSAS

SEQ ID NO: 131 - protein, 7 aa, Artificial Sequence
  Synthetic peptide
ATSELAS

SEQ ID NO: 132 - protein, 7 aa, Artificial Sequence
  Synthetic peptide
ATSTLAS

SEQ ID NO: 133 - protein, 9 aa, Artificial Sequence
  Synthetic peptide
PRRAPLPPT

SEQ ID NO: 134 - protein, 9 aa, Artificial Sequence
  Synthetic peptide
QAWVFPPPT

SEQ ID NO: 135 - protein, 9 aa, Artificial Sequence
  Synthetic peptide
QTWSSTPPT

SEQ ID NO: 136 - protein, 9 aa, Artificial Sequence
  Synthetic peptide
QPWSSNPPT

SEQ ID NO: 137 - protein, 5 aa, Artificial Sequence
  Synthetic peptide
NYAMN

SEQ ID NO: 138 - protein, 5 aa, Artificial Sequence
  Synthetic peptide
HEESQ

SEQ ID NO: 139 - protein, 5 aa, Artificial Sequence
  Synthetic peptide
RKRHN

SEQ ID NO: 140 - protein, 5 aa, Artificial Sequence
  Synthetic peptide
WRAIN

SEQ ID NO: 141 - protein, 5 aa, Artificial Sequence
  Synthetic peptide
AVAWN

SEQ ID NO: 142 - protein, 5 aa, Artificial Sequence
  Synthetic peptide
NYAKN

SEQ ID NO: 143 - protein, 10 aa, Artificial Sequence
  Synthetic peptide
RIRSKSNNYA

SEQ ID NO: 144 - protein, 10 aa, Artificial Sequence
  Synthetic peptide
YSSKXSTTYA

SEQ ID NO: 145 - protein, 10 aa, Artificial Sequence
  Synthetic peptide
HXRSKSRYYA

SEQ ID NO: 146 - protein, 10 aa, Artificial Sequence
  Synthetic peptide
RIRSKSNSYA

SEQ ID NO: 147 - protein, 9 aa, Artificial Sequence
  Synthetic peptide
TFYADSVKD

SEQ ID NO: 148 - protein, 9 aa, Artificial Sequence
  Synthetic peptide
SRLTPSPFD

SEQ ID NO: 149 - protein, 9 aa, Artificial Sequence
  Synthetic peptide
RHYKKSRKD

SEQ ID NO: 150 - protein, 12 aa, Artificial Sequence
  Synthetic peptide
RGTSSHKKHKAM

SEQ ID NO: 151 - protein, 12 aa, Artificial Sequence
  Synthetic peptide
RGASGIDYCHDY

SEQ ID NO: 152 - protein, 12 aa, Artificial Sequence
  Synthetic peptide
RGASGIDYANDY

SEQ ID NO: 153 - protein, 12 aa, Artificial Sequence
  Synthetic peptide
RGASGIDYALDY

SEQ ID NO: 154 - protein, 10 aa, Artificial Sequence
  Synthetic peptide
RASSSVXYMH

SEQ ID NO: 155 - protein, 9 aa, Artificial Sequence
  Synthetic peptide
QQWXXNPPT

SEQ ID NO: 156 - protein, 15 aa, Artificial Sequence
  Synthetic peptide
GGGGSGGGGSGGGGS

(19) United States
(12) Patent Application Publication
(10) Pub. No.: US 2010/0260752 A1
(43) Pub. Date: Oct. 14, 2010

(54) OPSONIC AND PROTECTIVE ANTIBODIES SPECIFIC FOR LIPOTEICHOIC ACID OF GRAM POSITIVE BACTERIA

(75) Inventor: Ying TANG, San Diego, CA (US)

Correspondence Address: LAHIVE & COCKFIELD, LLP;FLOOR 30, SUITE 3000, ONE POST OFFICE SQUARE, BOSTON, MA, 02109, US

(73) Assignee: Biosynexus Incorporated, Gaithersburg, MD (US)

(21) Appl. No.: 12/691,908

(22) Filed: Jan. 22, 2010

Related U.S. Application Data
(60) Provisional application No. 61/146,894, filed on Jan. 23, 2009.

(51) Int. Cl. A61K 39/395; C07K 16/12; C07H 21/04; C07K 7/08; C07K 7/06; A61P 31/04; C12N 5/07; C12N 5/10; A61K 39/40; C12N 15/63

(52) U.S. Cl. 424/133.1; 530/389.5; 530/388.4; 530/387.3; 530/391.1; 536/23.5; 530/326; 530/327; 530/328; 530/329; 435/340; 435/328; 424/164.1; 424/150.1; 435/320.1

(57) ABSTRACT

This invention provides binding molecules with improved binding affinity to lipoteichoic acids exposed on the surface of the bacteria, useful in the prevention and treatment of infections caused by Gram positive bacteria.

### RELATED APPLICATION

This patent application claims the benefit of U.S. Provisional Patent Application Ser. No. 61/146894, entitled “OPSONIC AND PROTECTIVE ANTIBODIES SPECIFIC FOR LIPOTEICHOIC ACID OF GRAM POSITIVE BACTERIA”, filed Jan. 23, 2009. The entire contents of the above-referenced provisional patent application are incorporated herein by reference.

### BACKGROUND OF THE INVENTION

The number of both community acquired and hospital acquired infections have increased over recent years with the increased use of intravascular devices. Hospital acquired (nosocomial) infections are a major cause of morbidity and mortality, more particularly in the US, where they affect more than 2 million patients annually. Following various studies, about 6 percent of the US patients will acquire an infection during their stay in hospital.

Staphylococcus aureus, Coagulase-negative Staphylococci (mostly Staphylococcus epidermidis), Enterococcus spp, Esherichia coli and Pseudomonas aeruginosa are the major nosocomial pathogens. Although those pathogens almost cause the same number of infections, the severity of the disorders they can produce combined with the frequency of antibiotic resistant isolates balance this ranking towards S. aureus and S. epidermidis as being the most significant nosocomial pathogens. Infections frequently occur in premature infants that have received parenteral nutrition which can be a direct or indirect source of contamination.

Staphylococcus aureus is the most common cause of nosocomial infections with a significant morbidity and mortality (Romero-Vivas et al. 1995, Infect. Dis. 2 1; 1417). It is the cause of some cases of sepsis in neonates, osteomyelitis, endocarditis, septic arthritis, pneumonia, abscesses and toxic shock syndrome.

S. epidermidis is a normal skin commensal which is also an important opportunistic pathogen responsible for infections of implanted medical devices and infections at sites of surgery. Medical devices infected by S. epidermidis include cardiac pacemakers, cerebrospinal fluid shunts, continuous ambulatory peritoneal dialysis catheters, orthopaedic devices and prosthetic heart valves.

S. aureus and S. epidermidis infections are treated with antibiotics, with penicillin being the drug of choice whereas vancomycin is used for methicillin resistant isolates. The percentage of staphylococcal strains exhibiting wide-spectrum resistance to antibiotics has become increasingly prevalent since the 1980's (Panlilo et al. (1992) Infect. Control. Hosp. Epidemiol. 13; 582), posing a threat for effective antimicrobial therapy. In addition, the recent emergence of vancomycin resistant S. aureus strain has aroused fear that methicillin resistant S. aureus strains will emerge and spread for which no effective therapy is available.

An alternative approach of using antibodies against staphylococcal antigens in the prevention and treatment of infection has been investigated. Therapy involving administration of polyclonal antisera are under development (WO 00/15238, WO 00/12132) as well as prevention and treatment with a monoclonal antibody against lipoteichoic acid, pagibaximab (WO 98/57994).

While pagibaximab has shown success in clinical studies, optimized variants of LTA antibodies having increased binding affinity would be beneficial. The production of optimized antibodies (i.e., antibodies with high biological activity, such as antigen neutralizing ability), including antibodies with high affinity for the target antigen, would be desirable from the point of view of both the neutralizing ability of such an antibody as well as from the more practical aspects of requiring less antibody in order to achieve a desirable degree of clinical effectiveness, thereby cutting costs of use.

### SUMMARY OF THE INVENTION

In one aspect, the invention provides a lipoteichoic acid (LTA) binding molecule, comprising a light chain comprising three complementarity determining regions (CDRs) from a parent A110 antibody light chain variable region set forth as SEQ ID NO: 1, and a heavy chain comprising three complementarity determining regions (CDRs) from a parent A110 antibody heavy chain variable region set forth as SEQ ID NO: 2, wherein at least one amino acid residue within the light or heavy chain CDRs, or both, is modified as compared to the parent, wherein said modified amino acid residue is selected from the group consisting of: 31L, 92L, 93L, 31H, 52cH, 61H, 98H and 100aH, according to Kabat numbering, and combinations thereof, provided that said binding molecule does not comprise the amino acid sequence set forth as SEQ ID NO:45 or SEQ ID NO:46. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one embodiment, the modified amino acid residue is 98H, 100aH or both 98H and 100aH. In another embodiment, the binding molecule further comprises a modified amino acid residue at 31L. In another embodiment, the binding molecule further comprises a modified amino acid residue at 31H. In another embodiment, the binding molecule further comprises a modified amino acid residue at 93L. In another embodiment, the binding molecule further comprises a modified amino acid residue at 92L and 52cH.

In one embodiment, the modified amino acid residues are 31L, 93L and 98H. In another embodiment, the modified amino acid residues are 31L, 93L, 98H and 100aH. In another embodiment, the modified amino acid residues are 31L, 92L, 93L and 52cH. In another embodiment, the modified amino acid residues are 92L, 93L, 52cH and 100aH. In another embodiment, the modified amino acid residues are 31L, 93L, 31H and 52cH. In another embodiment, the modified amino acid residues are 31L, 93L, 52cH and 98H. In another embodiment, the modified amino acid residues are 31L, 93L, 52cH and 100aH. In another embodiment, the modified amino acid residues are 31L, 93L, 31H, 52cH and 98H. In another embodiment, the modified amino acid residues are 31L, 93L, 31H, 52cH and 100aH. In another embodiment, the modified amino acid residues are 31L, 93L, 52cH, 98H and 100aH. In another embodiment, the modified amino acid residues are 31L, 93L, 31H, 98H and 100aH.

In one embodiment, the modified amino acid residue is a positively charged amino acid residue.

In one embodiment, amino acid residue 31L is Arg. In one embodiment, amino acid residue 92L is Arg. In one embodiment, amino acid residue 93L is Tyr or Lys. In another embodiment, amino acid residue 31H is Lys. In another embodiment, amino acid residue 52cH is Lys or Arg. In another embodiment, amino acid residue 98H is Arg or Lys. In one embodiment, amino acid residue 100aH is selected from the group consisting of His, Asn, Ala and Arg. In one embodiment, acid residue 98H is Arg and amino acid residue 100aH is His. In one embodiment, amino acid residue 92L is Arg and amino acid residue 93L is Tyr. In one embodiment, amino acid residue 92L is Arg and amino acid residue 93L is Lys.

In one embodiment, the binding molecule has a 5-fold increased binding affinity for LTA as compared to the parent antibody.

In another aspect, the invention provides a lipoteichoic acid (LTA) binding molecule, comprising a light chain comprising three complementarity determining regions (CDRs) from a parent A110 antibody light chain variable region set forth as SEQ ID NO: 1, and a heavy chain comprising three complementarity determining regions (CDRs) from a parent A110 antibody heavy chain variable region set forth as SEQ ID NO: 2, wherein at least one amino acid residue within the heavy chain CDR3 and at least one amino acid residue within the light chain CDR1 is modified as compared to the parent, provided that said binding molecule is not SEQ ID NO:45 or SEQ ID NO:46. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one embodiment, the modified amino acid residue within the heavy chain CDR3 is 98H, 100aH, or both 98H and 100aH. In one embodiment, the modified amino acid residue within the light chain CDR1 is 31L. In one embodiment, the modified amino acid residue 98H is Arg or Lys. In one embodiment, the modified amino acid residue 100aH is selected from the group consisting of His, Asn, Ala and Arg. In one embodiment, the modified amino acid residue 31L is Arg.

In another aspect, the invention provides a lipoteichoic acid (LTA) binding molecule, comprising a light chain comprising three complementarity determining regions (CDRs) from a parent A110 antibody light chain variable region set forth as SEQ ID NO: 1, and a heavy chain comprising three complementarity determining regions (CDRs) from a parent A110 antibody heavy chain variable region set forth as SEQ ID NO: 2, wherein at least one amino acid residue within the heavy chain CDR3 and at least one amino acid residue within the heavy chain CDR1 is modified as compared to the parent, provided that said binding molecule is not SEQ ID NO:45 or SEQ ID NO:46. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one embodiment, the modified amino acid residue within the heavy chain CDR3 is 98H, 100aH, or both 98H and 100aH. In one embodiment, the modified amino acid residue within the heavy chain CDR1 is 31H. In one embodiment, the modified amino acid residue 98H is Arg or Lys. In one embodiment, the modified amino acid residue 100aH is selected from the group consisting of His, Asn, Ala and Arg. In another embodiment, the modified amino acid residue 31H is Lys.

In another aspect, the invention provides a lipoteichoic acid (LTA) binding molecule, comprising a light chain comprising three complementarity determining regions (CDRs) from a parent A110 antibody light chain variable region set forth as SEQ ID NO: 1, and a heavy chain comprising three complementarity determining regions (CDRs) from a parent A110 antibody heavy chain variable region set forth as SEQ ID NO: 2, wherein at least one amino acid residue within the light chain CDR3, at least one amino acid residue within the heavy chain CDR2, and at least one amino acid residue within the heavy chain CDR3 is modified as compared to the parent, provided that said binding molecule is not SEQ ID NO:45 or SEQ ID NO:46. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one embodiment, the modified amino acid residue within the light chain CDR3 is 93L. In one embodiment, the modified amino acid residue within the heavy chain CDR2 is 52cH, 61H, or both 52cH and 61H. In one embodiment, the modified amino acid residue within the heavy chain CDR3 is 98H, 100aH, or both 98H and 100aH. In another embodiment, the modified amino acid residue 93L is Lys or Tyr. In another embodiment, the modified amino acid residue 52cH is Lys or Arg. In another embodiment, the modified amino acid residue 61H is Pro. In one embodiment, the modified amino acid residue 98H is Arg or Lys. In one embodiment, the modified amino acid residue 100aH is selected from the group consisting of His, Asn, Ala and Arg.

In another aspect, the invention provides a lipoteichoic acid (LTA) binding molecule, comprising a light chain comprising three complementarity determining regions (CDRs) from the parent A110 antibody light chain variable region set forth as SEQ ID NO: 1, and a heavy chain comprising three complementarity determining regions (CDRs) from the parent A110 antibody heavy chain variable region set forth as SEQ ID NO: 2, wherein at least one amino acid residue within the light chain CDR1, at least one amino acid residue within the light chain CDR3, at least one amino acid residue within the heavy chain CDR2, and at least one amino acid residue within the heavy chain CDR3 is modified as compared to the parent, provided that said binding molecule is not SEQ ID NO:45 or SEQ ID NO:46. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one embodiment, the modified amino acid residue within the light chain CDR1 is 31L. In one embodiment, the modified amino acid residue within the light chain CDR3 is 93L. In another embodiment, the modified amino acid residue within the heavy chain CDR2 is 52cH, 61H, or both 52cH and 61H. In another embodiment, the modified amino acid residue within the heavy chain CDR3 is 98H, 100aH, or both 98H and 100aH. In one embodiment, the modified amino acid residue 31L is Arg. In one embodiment, the modified amino acid residue 93L is Lys or Tyr. In one embodiment, the modified amino acid residue 52cH is Lys or Arg. In one embodiment, the modified amino acid residue 61H is Pro. In one embodiment, the modified amino acid residue 98H is Arg or Lys. In one embodiment, the modified amino acid residue 100aH is selected from the group consisting of His, Asn, Ala and Arg.

In one embodiment, the binding molecule of the invention further comprises at least one additional amino acid residue within the heavy chain CDR3 which is modified as compared to the parent. In one embodiment, the at least one additional modified amino acid residue is selected from the group consisting of H54, H99 and H102. In one embodiment, the modified amino acid residue H54 is Arg. In another embodiment, the modified amino acid residue H99 is Ser or Lys. In another embodiment, the modified amino acid residue H102 is Lys.

In one aspect, the invention provides a monoclonal antibody which specifically binds lipoteichoic acid (LTA), or antigen-binding fragment thereof, wherein the antibody comprises a light chain comprising three complementarity determining regions

(CDRs) set forth as SEQ ID NO:14, SEQ ID NO:4, and SEQ ID NO:15. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one aspect, the invention provides a monoclonal antibody which specifically binds lipoteichoic acid (LTA), or antigen-binding fragment thereof, wherein the antibody comprises a light chain comprising three complementarity determining regions (CDRs) set forth as SEQ ID NO:14, SEQ ID NO:4, and SEQ ID NO:16. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a light chain comprising three complementarity determining regions (CDRs) set forth as SEQ ID NO:3, SEQ ID NO:4, and SEQ ID NO:17. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection. In another aspect, the invention provides a monoclonal antibody which specifically binds lipoteichoic acid (LTA), or antigen-binding fragment thereof, wherein the antibody comprises a light chain comprising three complementarity determining regions (CDRs) set forth as SEQ ID NO:14, SEQ ID NO:4, and SEQ ID NO:17. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a light chain comprising three complementarity determining regions (CDRs) set forth as SEQ ID NO:3, SEQ ID NO:4, and SEQ ID NO:18. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a light chain comprising three complementarity determining regions (CDRs) set forth as SEQ ID NO:14, SEQ ID NO:4, and SEQ ID NO:5. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:11, SEQ ID NO:7, and SEQ ID NO:19. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:7, and SEQ ID NO:19. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:20, SEQ ID NO:21, and SEQ ID NO:8. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:21, and SEQ ID NO:22. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:20, SEQ ID NO:21, and SEQ ID NO:23. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:21, and SEQ ID NO:19. In one embodiment, the invention provides the use of such a monoclonal antibody in therapy.

In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:20, SEQ ID NO:21, and SEQ ID NO:22. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:7, and SEQ ID NO:22. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:21, and SEQ ID NO:23. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:20, SEQ ID NO:7, and SEQ ID NO:19. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:21, and SEQ ID NO:8. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:24, and SEQ ID NO:22. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:7, and SEQ ID NO:25. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:7, and SEQ ID NO:26. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:27, and SEQ ID NO:23. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:7, and SEQ ID NO:28. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:20, SEQ ID NO:7, and SEQ ID NO:29. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:7, and SEQ ID NO:30. In one embodiment, the invention provides the use of such a monoclonal antibody in therapy.

In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:7, and SEQ ID NO:31. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a monoclonal antibody which specifically binds lipoteichoic acid (LTA), or antigen-binding fragment thereof, wherein the antibody comprises a heavy chain comprising three complementarity determining regions (CDRs) set forth as SEQ ID NO:6, SEQ ID NO:7, and SEQ ID NO:32. In one embodiment, the invention provides the use of such a monoclonal antibody in therapy. In another embodiment, the invention provides the use of such a monoclonal antibody in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one embodiment, the binding molecule, monoclonal antibody or antigen-binding fragment thereof, specifically binds whole bacteria. In another embodiment, the binding molecule, monoclonal antibody or antigen-binding fragment thereof is selected from the group consisting of: a whole antibody, an antibody fragment, a humanized antibody, a human antibody, a single chain antibody, an immunoconjugate, a defucosylated antibody, an aglycosylated antibody, and a bispecific antibody. In one embodiment, the antibody fragment is selected from the group consisting of a Fab fragment, a Fab′ fragment, a F(ab)₂fragment, and a Fᵥ fragment.

In one aspect, the invention provides a cell producing the binding molecule, monoclonal antibody or antigen-binding fragment thereof described herein.

In another aspect, the invention provides a composition comprising a binding molecule, monoclonal antibody or antigen-binding fragment thereof as described herein and a pharmaceutically acceptable carrier. In one embodiment, the invention provides the use of such a binding molecule, monoclonal antibody or antigen-binding fragment thereof as described herein in therapy. In another embodiment, the invention provides the use of such a binding molecule, monoclonal antibody or antigen-binding fragment thereof as described herein in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one aspect, the invention provides a method of preventing a Staphylococcal infection in a human comprising administering the composition of the invention to the human.

In another aspect, the invention provides an isolated nucleic acid of SEQ ID NOs:35, 36, 108, 109, 110, 111, 112, 113, 114 or 115. In another embodiment, the invention provides an expression vector comprising such nucleic acids. In another aspect, the invention provides a cell comprising such expression vectors. In another embodiment, the invention provides an isolated nucleic acid molecule which corresponds to the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31 and SEQ ID NO:32.

In another aspect, the invention provides an isolated peptide, wherein the peptide comprises an amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31 and SEQ ID NO:32.

In one aspect, the invention provides a binding molecule that specifically binds lipoteichoic acid (LTA), wherein the binding molecule comprises a light chain and a heavy chain, the light chain comprising three variable region complementarity determining regions (CDRs), wherein a) CDR1 comprises the amino acid sequence: Arg Ala Ser Ser Ser Val Xaa₁ Tyr Met His (SEQ ID NO: 116); b) CDR2 comprises the amino acid sequence: Ala Thr Ser Asn Leu Ala Ser (SEQ ID NO: 117); c) CDR3 comprises the amino acid sequence: Gln Gln Trp Xaa₂ Xaa₃ Asn Pro Pro Thr (SEQ ID NO: 118); wherein Xaa₁, Xaa₂, Xaa₃ are any amino acid, provided that where CDR1 is SEQ ID NO:3 or SEQ ID NO:9, then CDR3 is not SEQ ID NO:5, and where CDR3 is SEQ ID NO:5, then CDR1 is not SEQ ID NO:3 or SEQ ID NO:9. In another aspect, the invention provides a binding molecule that specifically binds lipoteichoic acid (LTA), wherein the antibody comprises a light chain and a heavy chain, the heavy chain comprising three variable region complementarity determining regions (CDRs), wherein a) CDR1 comprises the amino acid sequence: Xaa₄ Tyr Ala Met Asn (SEQ ID NO: 119); b) CDR2 comprises the amino acid sequence: Arg Ile Arg Ser Lys Xaa₅ Asn Xaa₆ Tyr Ala Thr Xaa₇ Tyr Ala Asp Ser Val Lys Asp (SEQ ID NO: 120); c) CDR3 comprises the amino acid sequence: Arg Gly Xaa₈ Xaa₉ Xaa₁₀ Xaa₁₁ Xaa₁₂ Tyr Ala Met Asp Xaa₁₃ (SEQ ID NO: 121); wherein Xaa₄, Xaa₅, Xaa₆, Xaa₇, Xaa₈, Xaa₉, Xaa₁₀, Xaa₁₁, Xaa₁₂, and Xaa₁₃ are any amino acid, provided that i) where CDR1 is SEQ ID NO:6, then CDR2 is not SEQ ID NO:7 and CDR3 is not SEQ ID NO:8; ii) where CDR2 is SEQ ID NO:7 and CDR3 is SEQ ID NO:8, then CDR1 is not SEQ ID NO:6; iii) where CDR1 is SEQ ID NO:11, then CDR3 is not SEQ ID NO:13; and iv) where CDR3 is SEQ ID NO:13, then CDR 1 is not SEQ ID NO:11. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one embodiment, the binding molecule is an antibody, fragment thereof, or antigen binding fragment thereof. In one embodiment, Xaa₁, Xaa₂, Xaa₃, Xaa₄, Xaa₅, Xaa₆, Xaa₉, Xaa₁₀, or Xaa₁₃is a positively-charged amino acid. In another embodiment, Xaa₁ is Asn or Arg. In another embodiment, Xaa₁ is Arg. In another embodiment, Xaa₂ is Ser or Arg. In yet another embodiment, Xaa₃ is Ser, Tyr or Lys. In another embodiment, Xaa₃ is Tyr or Lys. In another embodiment, Xaa₃ is Tyr. In yet another embodiment, Xaa₃ is Lys. In yet another embodiment, Xaa₄ is Lys, Xaa₅ is Lys or Ser, Xaa₉ is Arg, and Xaa₁₂ is Asp or His. In one embodiment, Xaa₅ is Lys. In another embodiment, Xaa₅ is Ser. In another embodiment, Xaa₁₂ is Asp. In yet another embodiment, Xaa₁₂ is His.

In another aspect, the invention provides a binding molecule that specifically binds lipoteichoic acid (LTA), wherein the immunoglobulin comprises a light chain and a heavy chain, the light chain comprising three variable region complementarity determining regions (CDRs), wherein a) CDR1 comprises the amino acid sequence: Arg Ala Ser Ser Ser Val Xaa₁ Tyr Met His (SEQ ID NO: 154); b) CDR2 comprises the amino acid sequence: Ala Thr Ser Asn Leu Ala Ser (SEQ ID NO: 117); c) CDR3 comprises the amino acid sequence: Gln Gln Trp Xaa₂ Xaa₃ Asn Pro Pro Thr (SEQ ID NO: 155); wherein Xaa₁ is Asn, Arg or Ser, Xaa₂ is Ser or Arg, and Xaa₃ is Ser, Lys, or Tyr; provided that where CDR1 is SEQ ID NO:3 or SEQ ID NO:9, then CDR3 is not SEQ ID NO:5, and where CDR3 is SEQ ID NO:5, then CDR1 is not SEQ ID NO:3 or SEQ ID NO:9. In one embodiment, the binding molecule is an antibody, fragment thereof, or antigen binding fragment thereof. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a binding molecule that specifically binds lipoteichoic acid (LTA), wherein the antibody comprises a light chain and a heavy chain, the heavy chain comprising three variable region complementarity determining regions (CDRs), wherein a) CDR1 comprises the amino acid sequence: Gly Phe Thr Phe Asn Xaa₄ Tyr Ala Met Asn (SEQ ID NO: 122); b) CDR2 comprises the amino acid sequence: Arg Ile Arg Ser Lys Xaa₅ Asn Xaa₆ Tyr Ala Thr Xaa₇ Tyr Ala Asp Ser Val Lys Asp (SEQ ID NO: 123); c) CDR3 comprises the amino acid sequence: Arg Gly Xaa₈ Xaa₉ Xaa₁₀ Xaa₁₁ Xaa₁₂ Tyr Ala Met Asp Xaa₁₃ (SEQ ID NO: 124); wherein Xaa₄ is Asn, Thr, or Lys, Xaa₅ is Ser, Lys, or Arg, Xaa₆ is Arg or Asn, Xaa₇ is Phe or Tyr, Xaa₈ is Ala or Gly, Xaa₉ is Ser, Lys or Arg, Xaa₁₀ is Gly, Glu, Ser, or Lys, Xaa₁₁ is Ile or Thr, Xaa₁₂ is Asp, His, Asn, Ala or Arg, and Xaa₁₃ is Tyr or Lys, provided that i) where CDR1 is SEQ ID NO:6, then CDR2 is not SEQ ID NO:7 and CDR3 is not SEQ ID NO:8; ii) where CDR2 is SEQ ID NO:7 and CDR3 is SEQ ID NO:8, then CDR1 is not SEQ ID NO:6; iii) where CDR1 is SEQ ID NO:11, then CDR3 is not SEQ ID NO:13; and iv) where CDR3 is SEQ ID NO:13, then CDR 1 is not SEQ ID NO:11. In one embodiment, the binding molecule is an antibody, fragment thereof, or antigen binding fragment thereof. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In another aspect, the invention provides a lipoteichoic acid (LTA) binding molecule, comprising: a light chain comprising three complementarity determining regions (CDRs) from the parent A110 antibody light chain variable region set forth as SEQ ID NO: 1, and a heavy chain comprising three complementarity determining regions (CDRs) from the parent A110 antibody heavy chain variable region set forth as SEQ ID NO: 2, wherein at least one amino acid residue within the heavy chain CDR3 is modified as compared to the parent, wherein said amino acid residue is 98H or 100aH, and combinations thereof, provided that said binding molecule is not SEQ ID NO:45 or SEQ ID NO:46. In one embodiment, amino acid residue 98H is Arg or Lys. In another embodiment, amino acid residue 100aH is selected from the group consisting of His, Asn, Ala and Arg. In yet another embodiment, amino acid residue 98H is Arg and amino acid residue 100aH is His. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection. In yet another aspect, the invention provides a lipoteichoic acid (LTA) binding molecule, comprising: a light chain comprising three complementarity determining regions (CDRs) from the parent A110 antibody light chain variable region set forth as SEQ ID NO: 1, and a heavy chain comprising three complementarity determining regions (CDRs) from the parent A110 antibody heavy chain variable region set forth as SEQ ID NO: 2, wherein at least one amino acid residue within the heavy chain CDR3 is different from the parent, provided that said binding molecule is not SEQ ID NO:45 or SEQ ID NO:46. In one embodiment, the amino acid residue is 98H or 100aH, and combinations thereof. In another embodiment, the amino acid residue 98H is Arg or Lys. In yet another embodiment, the amino acid residue 100aH is His or Asn or Ala or Arg. In one embodiment, the invention provides the use of such an LTA binding molecule in therapy. In another embodiment, the invention provides the use of such an LTA binding molecule in the manufacture of a medicament for the treatment of a disease or disorder associated with a Gram positive bacterial infection, a S. aureus bacterial infection, a S. epidermidis bacterial infection, a coagulase negative staphylococci bacterial infection, or a Streptococcus mutans bacterial infection.

In one embodiment of the invention, the antibody fragment is selected from the group consisting of: a UniBody, a domain antibody, and a Nanobody.

In another aspect, the invention encompasses each of the sequences described in the Sequence Listing Table or Tables 1-11, individually or in combination, provided that the sequence is not the parent A110 antibody or the A120 antibody.

### BRIEF DESCRIPTION OF THE DRAWINGS

FIGS. 1A and 1B depict the correlation of LTA binding to live cell assay. Four strains of bacteria were utilized, S. epidermidis strain Hay, SE1175 (S. epidermidis clinical isolate), SE360 (S. epidermidis clinical isolate) and SE4928 (S. epidermidis clinical isolate).

FIG. 2 depicts results from the LTA binding assay of HCDR3 beneficial variants in anti-Fab capture format.

FIG. 3 depicts titration results of select HCDR3 variants on LTA.

FIG. 4 depicts titration results of the HCDR3 variants A1, C10, D3 and G10on live bacteria strain SE4928 (S. epidermidis clinical isolate).

FIG. 5 depicts titration results of the HCDR3 variants A1, C10, D3 and G10on live bacteria strain SE360 (S. epidermidis clinical isolate).

FIG. 6 depicts titration results of the HCDR3 variants A1, C10, D3 and G10 on live bacteria strain SE1175 (S. epidermidis clinical isolate).

FIG. 7 depicts titration results of the HCDR3 variants A1, C10, D3 and G10on live bacteria S. epidermidis strain Hay.

FIG. 8 depicts titration results of select combinatorial variants on live bacteria strain SE4555 (S. epidermidis clinical isolate).

FIG. 9 depicts titration results of select combinatorial variants on live bacteria strain SE6895 (S. epidermidis clinical isolate).

FIG. 10 depicts titration results of select combinatorial variants on live bacteria strain SE688 (S. epidermidis clinical isolate).

FIG. 11 depicts titration results of select combinatorial variants on live bacteria strain SE3827 (S. epidermidis clinical isolate).

FIG. 12 depicts titration results of select combinatorial variants on live bacteria strain SE380 (S. epidermidis clinical isolate).

FIG. 13 depicts titration results of select combinatorial variants on live bacteria strain SE10326 (S. epidermidis clinical isolate).

FIG. 14 depicts titration results of select combinatorial variants on live bacteria strain SE9294 (S. epidermidis clinical isolate).

FIG. 15 depicts titration results of select combinatorial variants on live bacteria, S. epidermidis strain Hay.

FIG. 16 depicts titration results of select combinatorial variants on live S. aureus bacteria strain SA5-Lab (S. aureus capsular type 5).

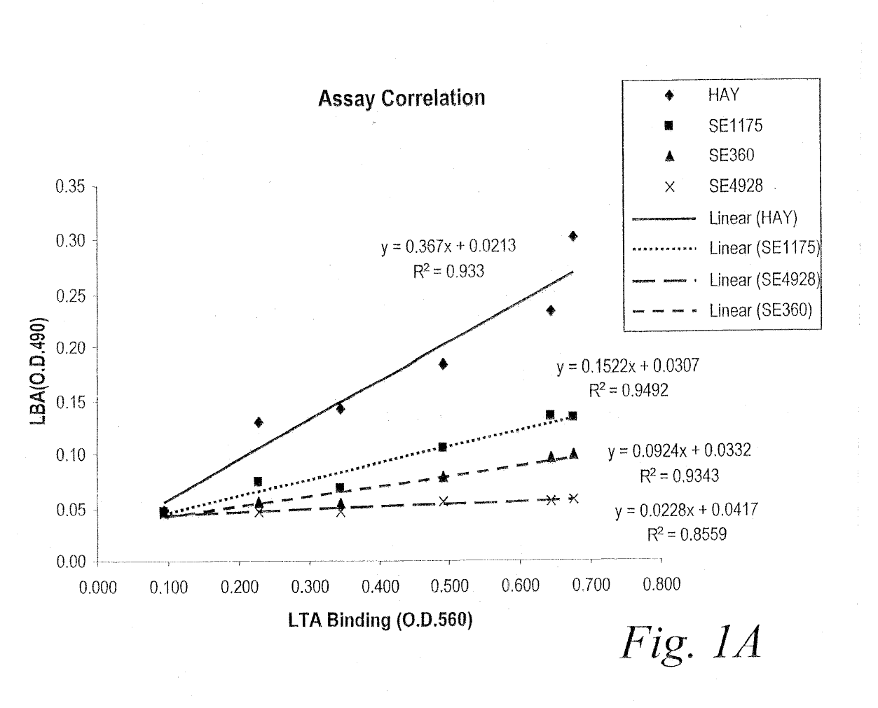

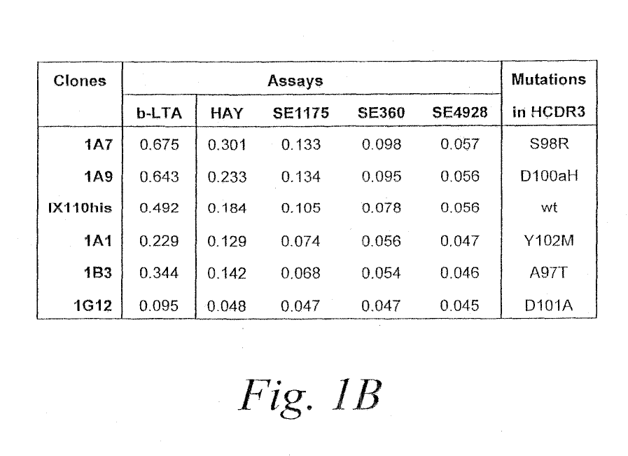

### DETAILED DESCRIPTION OF THE INVENTION

### Sequence Identification Numbers

Nucleotide and amino acid sequences referred to in the specification have been given the following sequence identification numbers provided in the Sequence Listing Table.

Sequence Listing Table

| | | | |
|---|---|---|---|
| SEQ | | | |
| ID | Present In | | |
| NO: | (Name) | Region | Sequence |

| | | | |
|---|---|---|---|
| 1 | A110 Parent | Light Chain | DIVLSQSPAILSASPGEKVTMTCRASSSVNYMHWYQ |
| | | Variable | QKPGSSPKPWISATSNLASGVPARFSGSGSGTSYSLT |
| | | Region | ISRVEAEDAATYYCQQWSSNPPTFGGGTMLEIK |
| 2 | A110 Parent | Heavy Chain | EVMLVESGGGLVQPKGSLKLSCAASGFTFNNYAMN |
| | | Variable | WVRQAPGKGLEWVARIRSKSNNYATFYADSVKDR |
| | | Region | FTISRDDSQSMLYLQMNNLKTEDTAMYYCVRRGAS |
| | | | GIDYAMDYWGQGTSLTVSS |
| 3 | A110 Parent, | Light Chain | RASSSVNYMH |
| | Com2A8, A1, | CDR1 | |
| | B6, B7, G10, | | |
| | 1D3, 4B2 | | |
| 4 | A110 Parent, | Light Chain | ATSNLAS |
| | A120, Com2B8, | CDR2 | |
| | Com1G2, | | |
| | Com2C7, | | |
| | Com2H1, | | |
| | Com2G4, | | |
| | Com2E1, | | |
| | Com1B4, | | |
| | Com2C2, | | |
| | Com2C5, | | |
| | Com2B11, | | |
| | Com1B12, | | |
| | Com2A8, A1, | | |
| | B6, B7, C10, | | |
| | D3, G10, 1D3, | | |
| | 4B2 | | |
| 5 | A110 Parent, | Light Chain | QQWSSNPPT |
| | A120, B6, B7, | CDR3 | |
| | D3, G10, 1D3, | | |
| | 4B2, 5A11 | | |
| 6 | A110 Parent, | Heavy Chain | GFTFNNYAMN |
| | Com2B8, | CDR1 | |
| | Com2C7, | | |
| | Com2G4, | | |
| | Com1B4, | | |
| | Com2C2, | | |
| | Com2C5, | | |
| | Com1B12, | | |
| | Com2A8, A1, | | |
| | B6, B7, C10, | | |
| | D3, 1D3, 4B2 | | |
| 7 | A110 Parent, | Heavy Chain | RIRSKSNNYATFYADSVKD |
| | Com2B8, | CDR2 | |
| | Com1B4, | | |
| | Com2C2, | | |
| | Com2B11, B6, | | |
| | B7, D3, G10, | | |
| | 1D3, 4B2 | | |
| 8 | A110 Parent, | Heavy Chain | RGASGIDYAMDY |
| | Com1G2, | CDR3 | |
| | Com1B12 | | |
| 9 | A120 | Light Chain | RASSSVSYMH |
| | | CDR1 | |
| 10 | A120 | Light Chain | QQWSSNPPT |
| | | CDR3 | |
| 11 | A120 | Heavy Chain | GFTFNTYAMN |
| | | CDR1 | |
| 12 | A120 | Heavy Chain | RIRSKSNNYATYYADSVKD |
| | | CDR2 | |
| 13 | A120 | Heavy Chain | RGGKETDYAMDY |
| | | CDR3 | |
| 14 | L1-C10 | LCDR1- | RASSSVRYMH |
| | Com2B8, | N31R | |
| | Com1G2, | | |
| | Com2C7, | | |
| | Com2H1, | | |
| | Com2G4, | | |
| | Com2E1, | | |
| | Com1B4, | | |
| | Com2C2, | | |
| | Com2C5, | | |
| | Com2B11, | | |
| | Com1B12, C10, | | |
| | D3 | | |
| 15 | Com2B8, | LCDR3- | QQWSYNPPT |
| | Com1G2, | S93Y | |
| | Com2C7, | | |
| | Com2H1, | | |
| | Com2G4, | | |
| | Com2E1, C10 | | |
| 16 | L3-B2 | LCDR3- | QQWSKNPPT |
| | Com2C2, | S93K | |
| | Com2C5, | | |
| | Com2B11 | | |
| 17 | Com1B12, | LCDR3- | QQWRKNPPT |
| | Com2A8 | S92R, S93K | |
| 18 | A1 | LCDR3- | QQWRSNPPT |
| | | S92R | |
| 19 | Com2B8, | HCDR3- | RGARGIHYAMDY |
| | Com2G4, | S98R, | |
| | Com2C2, | D100aH | |
| | Com2B11 | | |
| 20 | H2a-B3 | HCDR1- | GFTFNKYAMN |
| | Com1G2, | N30K | |
| | Com2H1, | H1-B3 | |
| | Com2E1, | | |
| | Com2B11, G10 | | |
| 21 | Com1G2, | HCDR2- | RIRSKKNNYATFYADSVKD |
| | Com2C7, | S52cK | |
| | Com2H1, | | |
| | Com2G4, | | |
| | Com2E1, | | |
| | Com2C5, | | |
| | Com1B12, | | |
| | Com2A8 | | |
| 22 | H3-A1 | HCDR3- | RGARGIDYAMDY |
| | Com2C7, | S98R | |
| | Com2E1, | | |
| | Com1B4, A1 | | |
| 23 | Com2H1, | HCDR3- | RGASGIHYAMDY |
| | Com2C5, | D100aH | |
| | Com2A8, C10 | | |
| 24 | H2a-A1 | HCDR2- | RIRSKSNRYATFYADSVKD |
| | | N54R | |
| 25 | H3-B6 | HCDR3- | RGAKGIDYAMDY |
| | | S98K | |
| 26 | H3-B7 | HCDR3- | RGASSIDYAMDY |
| | | G99S | |
| 27 | H2a-C10 | HCDR2- | RIRSKRNNYATFYADSVKD |
| | | S52cR | |
| 28 | H3-D3 | HCDR3- | RGASGINYAMDY |
| | | D100aN | |
| 29 | H3-G10 | HCDR3- | RGASKIDYAMDY |
| | | G99K | |
| 30 | H3-1D3 | HCDR3- | RGASGIAYAMDY |
| | | D100aA | |
| 31 | H3-4B2 | HCDR3- | RGASGIRYAMDY |
| | | D100aR | |
| 32 | H3-5A11 | HCDR3- | RGASGIDYAMDK |
| | | Y102K | |
| 33 | A110 Parent | Light Chain | DIVLSQSPAILSASPGEKVTMTCRASSSVNYMHWYQ |
| | | | QKPGSSPKPWISATSNLASGVPARFSGSGSGTSYSLT |
| | | | ISRVEAEDAATYYCQQWSSNPPTFGGGTMLEIKRTV |
| | | | AAPSVFIFPPSDEQLKSGTASVVCLLNNFYPREAKV |
| | | | QWKVDNALQSGNSQESVTEQDSKDSTYSLSSTLTLS |
| | | | KADYEKHKVYACEVTHQGLSSPVTKSFNRGEC |
| 34 | A110 Parent | Heavy Chain | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGAS |
| | | | GIDYAMDYWGQGTSLTVSSEFASTKGPSVFPLAPSS |
| | | | KSTSGGTAALGCLVKDYFPEPVTVSWNSGALTSGV |
| | | | HTFPAVLQSSGLYSLSSVVTVPSSSLGTQTYICNVNH |
| | | | KPSNTKVDKKVEPKSCDKTHTCPPCPAPELLGGPSV |
| | | | FLFPPKPKDTLMISRTPEVTCVVVDVSHEDPEVKFN |
| | | | WYVDGVEVHNAKTKPREEQYNSTYRVVSVLTVLH |
| | | | QDWLNGKEYKCKVSNKALPAPIEKTISKAKGQPRE |
| | | | PQVYTLPPSRDELTKNQVSLTCLVKGFYPSDIAVEW |
| | | | ESNGQPENNYKTTPPVLDSDGSFFLYSKLTVDKSRW |
| | | | QQGNVFSCSVMHEALHNHYTQKSLSLSPGK |
| 35 | A110 | Heavy Chain | GAAGTGATGCTGGTGGAGTCTGGTGGAGGATTGG |
| | | Variable | TGCAGCCTAAAGGGTCATTGAAACTCTCATGTGC |
| | | Region- | AGCCTCTGGATTCACCTTCAATAACTACGCCATG |
| | | Nucleic Acid | AATTGGGTCCGCCAGGCTCCAGGAAAGGGTTTGG |
| | | | AATGGGTTGCTCGCATAAGAAGTAAAAGTAATAA |
| | | | TTATGCAACATTTTATGCCGATTCAGTGAAAGAC |
| | | | AGGTTCACCATCTCCAGAGATGATTCACAAAGCA |
| | | | TGCTCTATCTGCAAATGAACAACTTGAAAACTGA |
| | | | GGACACAGCCATGTATTACTGTGTGAGACGGGGG |
| | | | GCTTCAGGGATTGACTATGCTATGGACTACTGGG |
| | | | GTCAAGGAACCTCACTCACCGTCTCCTCA |
| 36 | A110 | Light Chain | GATATCGTTCTCTCCCAGTCTCCAGCAATCCTGTC |
| | | Variable | TGCATCTCCAGGGGAAAAGGTCACAATGACTTGC |
| | | Region- | AGGGCCAGCTCAAGTGTAAATTACATGCACTGGT |
| | | Nucleic Acid | ACCAGCAGAAGCCAGGATCCTCCCCCAAACCCTG |
| | | | GATTTCTGCCACATCCAACCTGGCTTCTGGAGTCC |
| | | | CTGCTCGCTTCAGTGGCAGTGGGTCTGGGACCTCT |
| | | | TACTCTCTCACAATCAGCAGAGTGGAGGCTGAAG |
| | | | ATGCTGCCACTTATTACTGCCAGCAGTGGAGTAG |
| | | | TAACCCACCCACGTTCGGAGGGGGGACCATGCTG |
| | | | GAAATAAAA |
| 37 | A110 | Light Chain | DIVLSQSPAILSASPGEKVTMTC |
| | | Framework 1 | |
| 38 | A110 | Light Chain | WYQQKPGSSPKPWIS |
| | | Framework 2 | |
| 39 | A110 | Light Chain | GVPARFSGSGSGTSYSLTISRVEAEDAATYYC |
| | | Framework 3 | |
| 40 | A110 | Light Chain | FGGGTMLEIK |
| | | Framework 4 | |
| 41 | A110 | Heavy Chain | EVMLVESGGGLVQPKGSLKLSCAAS |
| | | Framework 1 | |
| 42 | A110 | Heavy Chain | WVRQAPGKGLEWVA |
| | | Framework 2 | |
| 43 | A110 | Heavy Chain | RFTISRDDSQSMLYLQMNNLKTEDTAMYYCVR |
| | | Framework 3 | |
| 44 | A110 | Heavy Chain | WGQGTSLTVSS |
| | | Framework 4 | |
| 45 | A120 | VL | DIVLSQSPAILSASPGEKVTMTCRASSSVSYMHWYQ |
| | | | QKPGSSPKPWIYATSNLASGVPARFSGSGSGTSYSLT |
| | | | ISRVEAEDAATYYCQQWSSNPPTFGGGTKLEIK |
| 46 | A120 | VH | EVMLVESGEGLVQPKGSLKLSCAASGFTFNTYAMN |
| | | | WVRQAPGKGLEWVARIRSKSNNYATYYADSVKDR |
| | | | FTISRDDSQSMLYLQMNNLKTEDTAMYYCVRRGG |
| | | | KETDYAMDYWGQGTSVTVSS |
| 47 | Com2B8 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSYNPPTFGGGTMLEIK |
| 48 | Com2B8 | VH | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGA |
| | | | RGIHYAMDYWGQGTSLTVSS |
| 49 | Com1G2 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSYNPPTFGGGTMLEIK |
| 50 | Com1G2 | VH | EVMLVESGGGLVQPKGSLKLSCAASGFTFNKYAM |
| | | | NWVRQAPGKGLEWVARIRSKKNNYATFYADSVKD |
| | | | RFTISRDDSQSMLYLQMNNLKTEDTAMYYCVRRG |
| | | | ASGIDYAMDYWGQGTSLTVSS |
| 51 | Com2C7 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSYNPPTFGGGTMLEIK |
| 52 | Com2C7 | VH | EVMLVESGGGLVQPKGSLKLSCAASGFTFNNYAMN |
| | | | WVRQAPGKGLEWVARIRSKKNNYATFYADSVKDR |
| | | | FTISRDDSQSMLYLQMNNLKTEDTAMYYCVRRGA |
| | | | RGIDYAMDYWGQGTSLTVSS |
| 53 | Com2H1 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSYNPPTFGGGTMLEIK |
| 54 | Com2H1 | VH | EVMLVESGGGLVQPKGSLKLSCAASGFTFNKYAM |
| | | | NWVRQAPGKGLEWVARIRSKKNNYATFYADSVKD |
| | | | RFTISRDDSQSMLYLQMNNLKTEDTAMYYCVRRG |
| | | | ASGIHYAMDYWGQGTSLTVSS |
| 55 | Com2G4 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSYNPPTFGGGTMLEIK |
| 56 | Com2G4 | VH | EVMLVESGGGLVQPKGSLKLSCAASGFTFNNYAMN |
| | | | WVRQAPGKGLEWVARIRSKKNNYATFYADSVKDR |
| | | | FTISRDDSQSMLYLQMNNLKTEDTAMYYCVRRGA |
| | | | RGIHYAMDYWGQGTSLTVSS |
| 57 | Com2E1 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSYNPPTFGGGTMLEIK |
| 58 | Com2E1 | VH | EVMLVESGGGLVQPKGSLKLSCAASGFTFNKYAM |
| | | | NWVRQAPGKGLEWVARIRSKKNNYATFYADSVKD |
| | | | RFTISRDDSQSMLYLQMNNLKTEDTAMYYCVRRG |
| | | | ARGIDYAMDYWGQGTSLTVSS |
| 59 | Com1B4 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSKNPPTFGGGTMLEIK |
| 60 | Com1B4 | VH | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGA |
| | | | RGIDYAMDYWGQGTSLTVSS |
| 61 | Com2C2 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSKNPPTFGGGTMLEIK |
| 62 | Com2C2 | VH | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGA |
| | | | RGIHYAMDYWGQGTSLTVSS |
| 63 | Com2C5 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSKNPPTFGGGTMLEIK |
| 64 | Com2C5 | VH | EVMLVESGGGLVQPKGSLKLSCAASGFTFNNYAMN |
| | | | WVRQAPGKGLEWVARIRSKKNNYATFYADSVKDR |
| | | | FTISRDDSQSMLYLQMNNLKTEDTAMYYCVRRGAS |
| | | | GIHYAMDYWGQGTSLTVSS |
| 65 | Com2B11 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSKNPPTFGGGTMLEIK |
| 66 | Com2B11 | VH | EVMLVESGGGLVQPKGSLKLSCAASGFTFNKYAM |
| | | | NWVRQAPGKGLEWVARIRSKSNNYATFYADSVKD |
| | | | RFTISRDDSQSMLYLQMNNLKTEDTAMYYCVRRG |
| | | | ARGIHYAMDYWGQGTSLTVSS |
| 67 | Com1B12 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWRKNPPTFGGGTMLEIK |
| 68 | Com1B12 | VH | EVMLVESGGGLVQPKGSLKLSCAASGFTFNNYAMN |
| | | | WVRQAPGKGLEWVARIRSKKNNYATFYADSVKDR |
| | | | FTISRDDSQSMLYLQMNNLKTEDTAMYYCVRRGAS |
| | | | GIDYAMDYWGQGTSLTVSS |
| 69 | Com2A8 | VL | DIVLSQSPAILSASPGEKVTMTCRASSSVNYMHWYQ |
| | | | QKPGSSPKPWISATSNLASGVPARFSGSGSGTSYSLT |
| | | | ISRVEAEDAATYYCQQWRKNPPTFGGGTMLEIK |
| 70 | Com2A8 | VH | EVMLVESGGGLVQPKGSLKLSCAASGFTFNNYAMN |
| | | | WVRQAPGKGLEWVARIRSKKNNYATFYADSVKDR |
| | | | FTISRDDSQSMLYLQMNNLKTEDTAMYYCVRRGAS |
| | | | GIHYAMDYWGQGTSLTVSS |
| 71 | A1 | VL | DIVLSQSPAILSASPGEKVTMTCRASSSVNYMHWYQ |
| | | | QKPGSSPKPWISATSNLASGVPARFSGSGSGTSYSLT |
| | | | ISRVEAEDAATYYCQQWRSNPPTFGGGTMLEIK |
| 72 | A1 | VH | EVMLVESGGGLVQPKGSLKLSCAASGFTFNNYAMN |
| | | | WVRQAPGKGLEWVARIRSKSNRYATFYADSVKDR |
| | | | FTISRDDSQSMLYLQMNNLKTEDTAMYYCVRRGA |
| | | | RGIDYAMDYWGQGTSLTVSS |
| 73 | B6 | VL | DIVLSQSPAILSASPGEKVTMTCRASSSVNYMHWYQ |
| | | | QKPGSSPKPWISATSNLASGVPARFSGSGSGTSYSLT |
| | | | ISRVEAEDAATYYCQQWSSNPPTFGGGTMLEIK |
| 74 | B6 | VH | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGA |
| | | | KGIDYAMDYWGQGTSLTVSS |
| 75 | B7 | VL | DIVLSQSPAILSASPGEKVTMTCRASSSVNYMHWYQ |
| | | | QKPGSSPKPWISATSNLASGVPARFSGSGSGTSYSLT |
| | | | ISRVEAEDAATYYCQQWSSNPPTFGGGTMLEIK |
| 76 | B7 | VH | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGAS |
| | | | SIDYAMDYWGQGTSLTVSS |
| 77 | C10 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSYNPPTFGGGTMLEIK |
| 78 | C10 | VH | EVMLVESGGGLVQPKGSLKLSCAASGFTFNNYAMN |
| | | | WVRQAPGKGLEWVARIRSKRNNYATFYADSVKDR |
| | | | FTISRDDSQSMLYLQMNNLKTEDTAMYYCVRRGAS |
| | | | GIHYAMDYWGQGTSLTVSS |
| 79 | D3 | VL | DIVLSQSPAILSASPGEKVTMTCRASSSVRYMHWYQ |
| | | | QKPGSSPKPWISATSNLASGVPARFSGSGSGTSYSLT |
| | | | ISRVEAEDAATYYCQQWSSNPPTFGGGTMLEIK |
| 80 | D3 | VH | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGAS |
| | | | GINYAMDYWGQGTSLTVSS |
| 81 | G10 | VL | DIVLSQSPAILSASPGEKVTMTCRASSSVNYMHWYQ |
| | | | QKPGSSPKPWISATSNLASGVPARFSGSGSGTSYSLT |
| | | | ISRVEAEDAATYYCQQWSSNPPTFGGGTMLEIK |
| 82 | G10 | VH | EVMLVESGGGLVQPKGSLKLSCAASGFTFNKYAM |
| | | | NWVRQAPGKGLEWVARIRSKSNNYATFYADSVKD |
| | | | RFTISRDDSQSMLYLQMNNLKTEDTAMYYCVRRG |
| | | | ASKIDYAMDYWGQGTSLTVSS |
| 83 | 1D3 | VL | DIVLSQSPAILSASPGEKVTMTCRASSSVNYMHWYQ |
| | | | QKPGSSPKPWISATSNLASGVPARFSGSGSGTSYSLT |
| | | | ISRVEAEDAATYYCQQWSSNPPTFGGGTMLEIK |
| 84 | 1D3 | VH | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGAS |
| | | | GIAYAMDYWGQGTSLTVSS |
| 85 | 4B2 | VL | DIVLSQSPAILSASPGEKVTMTCRASSSVNYMHWYQ |
| | | | QKPGSSPKPWISATSNLASGVPARFSGSGSGTSYSLT |
| | | | ISRVEAEDAATYYCQQWSSNPPTFGGGTMLEIK |
| 86 | 4B2 | VH | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGAS |
| | | | GIRYAMDYWGQGTSLTVSS |
| 87 | 5A11 | VL | DIVLSQSPAILSASPGEKVTMTCRASSSVNYMHWYQ |
| | | | QKPGSSPKPWISATSNLASGVPARFSGSGSGTSYSLT |
| | | | ISRVEAEDAATYYCQQWSSNPPTFGGGTMLEIK |
| 88 | 5A11 | VH | EVMLVESGGGLVQPKGSLKLSCAASGFTFNNYAMN |
| | | | WVRQAPGKGLEWVARIRSKSNNYATFYADSVKDR |
| | | | FTISRDDSQSMLYLQMNNLKTEDTAMYYCVRRGAS |
| | | | GIDYAMDKWGQGTSLTVSS |
| 89 | L1-A4 | LCDR1 | RASRSVNYMH |
| 90 | L1-E1 | LCDR1 | RASSSVKYMH |
| 91 | L2-A2 | LCDR2 | ATLNLAS |
| 92 | L2-A4 | LCDR2 | ATINLAS |
| 93 | L3-A1 | LCDR3 | QQWRSNPPT |
| 94 | L3-3C9 | LCDR3 | QQWSRNPPT |
| 95 | H1-C3 | HCDR1 | GFTFNRYAMN |
| 96 | H2a-C9 | HCDR2 | RIRSKSNKYATFYADSVKD |
| 97 | H2b-A4 | HCDR2 | RIRSKSNNYATFYAPSVKD |
| 98 | H3-C10 | HCDR3 | RGASGIHYAMDY |
| 99 | H3-D3 | HCDR3 | RGASGINYAMDY |
| 100 | A120 | Light Chain | DIVLSQSPAILSASPGEKVTMTC |
| | | Framework 1 | |
| 101 | A120 | Light Chain | WYQQKPGSSPKPWIY |
| | | Framework 2 | |
| 102 | A120 | Light Chain | GVPARFSGSGSGTSYSLTISRVEAEDAATYYC |
| | | Framework 3 | |
| 103 | A120 | Light Chain | FGGGTKLEIK |
| | | Framework 4 | |
| 104 | A120 | Heavy Chain | EVMLVESGEGLVQPKGSLKLSCAAS |
| | | Framework 1 | |
| 105 | A120 | Heavy Chain | WVRQAPGKGLEWVA |
| | | Framework 2 | |
| 106 | A120 | Heavy Chain | RFTISRDDSQSMLYLQMNNLKTEDTAMYYCVR |
| | | Framework 3 | |
| 107 | A120 | Heavy Chain | WGQGTSVTVSS |
| | | Framework 4 | |
| 108 | Com2E1 | Light Chain | GATATCGTTCTCTCCCAGTCTCCAGCAATCCTGTC |
| | | Variable | TGCATCTCCAGGGGAAAAGGTCACAATGACTTGC |
| | | Region- | AGGGCCAGCTCAAGTGTACGCTACATGCACTGGT |
| | | Nucleic Acid | ACCAGCAGAAGCCAGGATCCTCCCCCAAACCCTG |
| | | | GATTTCTGCCACATCCAACCTGGCTTCTGGAGTCC |
| | | | CTGCTCGCTTCAGTGGCAGTGGGTCTGGGACCTCT |
| | | | TACTCTCTCACAATCAGCAGAGTGGAGGCTGAAG |
| | | | ATGCTGCCACTTATTACTGCCAGCAGTGGAGTTAT |
| | | | AACCCACCCACGTTCGGAGGGGGGACCATGCTGG |
| | | | AAATAAAA |
| 109 | Com2E1 | Heavy Chain | GAAGTGATGCTGGTGGAGTCTGGTGGAGGATTGG |
| | | Variable | TGCAGCCTAAAGGGTCATTGAAACTCTCATGTGC |
| | | Region- | AGCCTCTGGATTCACCTTCAATAAGTACGCCATG |
| | | Nucleic Acid | AATTGGGTCCGCCAGGCTCCAGGAAAGGGTTTGG |
| | | | AATGGGTTGCTCGCATAAGAAGTAAAAAGAATAA |
| | | | TTATGCAACATTTTATGCCGATTCAGTGAAAGAC |
| | | | AGGTTCACCATCTCCAGAGATGATTCACAAAGCA |
| | | | TGCTCTATCTGCAAATGAACAACTTGAAAACTGA |
| | | | GGACACAGCCATGTATTACTGTGTGAGACGGGGG |
| | | | GCTCGTGGGATTGACTATGCTATGGACTACTGGG |
| | | | GTCAAGGAACCTCACTCACCGTCTCCTCA |
| 110 | Com2B11 | Light Chain | GATATCGTTCTCTCCCAGTCTCCAGCAATCCTGTC |
| | | Variable | TGCATCTCCAGGGGAAAAGGTCACAATGACTTGC |
| | | Region- | AGGGCCAGCTCAAGTGTACGCTACATGCACTGGT |
| | | Nucleic Acid | ACCAGCAGAAGCCAGGATCCTCCCCCAAACCCTG |
| | | | GATTTCTGCCACATCCAACCTGGCTTCTGGAGTCC |
| | | | CTGCTCGCTTCAGTGGCAGTGGGTCTGGGACCTCT |
| | | | TACTCTCTCACAATCAGCAGAGTGGAGGCTGAAG |
| | | | ATGCTGCCACTTATTACTGCCAGCAGTGGAGTAA |
| | | | GAACCCACCCACGTTCGGAGGGGGGACCATGCTG |
| | | | GAAATAAAA |
| 111 | Com2B11 | Heavy Chain | GAAGTGATGCTGGTGGAGTCTGGTGGAGGATTGG |
| | | Variable | TGCAGCCTAAAGGGTCATTGAAACTCTCATGTGC |
| | | Region- | AGCCTCTGGATTCACCTTCAATAAGTACGCCATG |
| | | Nucleic Acid | AATTGGGTCCGCCAGGCTCCAGGAAAGGGTTTGG |
| | | | AATGGGTTGCTCGCATAAGAAGTAAAAGTAATAA |
| | | | TTATGCAACATTTTATGCCGATTCAGTGAAAGAC |
| | | | AGGTTCACCATCTCCAGAGATGATTCACAAAGCA |
| | | | TGCTCTATCTGCAAATGAACAACTTGAAAACTGA |
| | | | GGACACAGCCATGTATTACTGTGTGAGACGGGGG |
| | | | GCTCGTGGGATTCACTATGCTATGGACTACTGGG |
| | | | GTCAAGGAACCTCACTCACCGTCTCCTCA |
| 112 | Com2C5 | Light Chain | GATATCGTTCTCTCCCAGTCTCCAGCAATCCTGTC |
| | | Variable | TGCATCTCCAGGGGAAAAGGTCACAATGACTTGC |
| | | Region- | AGGGCCAGCTCAAGTGTACGCTACATGCACTGGT |
| | | Nucleic Acid | ACCAGCAGAAGCCAGGATCCTCCCCCAAACCCTG |
| | | | GATTTCTGCCACATCCAACCTGGCTTCTGGAGTCC |
| | | | CTGCTCGCTTCAGTGGCAGTGGGTCTGGGACCTCT |
| | | | TACTCTCTCACAATCAGCAGAGTGGAGGCTGAAG |
| | | | ATGCTGCCACTTATTACTGCCAGCAGTGGAGTAA |
| | | | GAACCCACCCACGTTCGGAGGGGGGACCATGCTG |
| | | | GAAATAAAA |
| 113 | Com2C5 | Heavy Chain | GAAGTGATGCTGGTGGAGTCTGGTGGAGGATTGG |
| | | Variable | TGCAGCCTAAAGGGTCATTGAAACTCTCATGTGC |
| | | Region- | AGCCTCTGGATTCACCTTCAATAACTACGCCATG |
| | | Nucleic Acid | AATTGGGTCCGCCAGGCTCCAGGAAAGGGTTTGG |
| | | | AATGGGTTGCTCGCATAAGAAGTAAAAAGAATAA |
| | | | TTATGCAACATTTTATGCCGATTCAGTGAAAGAC |
| | | | AGGTTCACCATCTCCAGAGATGATTCACAAAGCA |
| | | | TGCTCTATCTGCAAATGAACAACTTGAAAACTGA |
| | | | GGACACAGCCATGTATTACTGTGTGAGACGGGGG |
| | | | GCTTCAGGGATTCACTATGCTATGGACTACTGGG |
| | | | GTCAAGGAACCTCACTCACCGTCTCCTCA |
| 114 | Com1G2 | Light Chain | GATATCGTTCTCTCCCAGTCTCCAGCAATCCTGTC |
| | | Variable | TGCATCTCCAGGGGAAAAGGTCACAATGACTTGC |
| | | Region- | AGGGCCAGCTCAAGTGTACGCTACATGCACTGGT |
| | | Nucleic Acid | ACCAGCAGAAGCCAGGATCCTCCCCCAAACCCTG |
| | | | GATTTCTGCCACATCCAACCTGGCTTCTGGAGTCC |
| | | | CTGCTCGCTTCAGTGGCAGTGGGTCTGGGACCTCT |
| | | | TACTCTCTCACAATCAGCAGAGTGGAGGCTGAAG |
| | | | ATGCTGCCACTTATTACTGCCAGCAGTGGAGTTAT |
| | | | AACCCACCCACGTTCGGAGGGGGGACCATGCTGG |
| | | | AAATAAAA |
| 115 | Com1G2 | Heavy Chain | GAAGTGATGCTGGTGGAGTCTGGTGGAGGATTGG |
| | | Variable | TGCAGCCTAAAGGGTCATTGAAACTCTCATGTGC |
| | | Region- | AGCCTCTGGATTCACCTTCAATAAGTACGCCATG |
| | | Nucleic Acid | AATTGGGTCCGCCAGGCTCCAGGAAAGGGTTTGG |
| | | | AATGGGTTGCTCGCATAAGAAGTAAAAAGAATAA |
| | | | TTATGCAACATTTTATGCCGATTCAGTGAAAGAC |
| | | | AGGTTCACCATCTCCAGAGATGATTCACAAAGCA |
| | | | TGCTCTATCTGCAAATGAACAACTTGAAAACTGA |
| | | | GGACACAGCCATGTATTACTGTGTGAGACGGGGG |
| | | | GCTTCAGGGATTGACTATGCTATGGACTACTGGG |
| | | | GTCAAGGAACCTCACTCACCGTCTCCTCA |

### I. Definitions

Prior to further describing the invention, it may be helpful to an understanding thereof to set forth definitions of certain terms to be used hereinafter.

As used herein, the terms “optimized antibody” and “mutant antibody,” used interchangeably herein, refer to an antibody having at least one amino acid which is different from the parent antibody in at least one complementarity determining region (CDR) in the light or heavy chain variable region, which confers a higher binding affinity, e.g., a 2-fold or more fold higher binding affinity, to the binding antigen as compared to the parent antibody.

The terms “LTA antibody” and “anti-LTA” are used interchangeably herein to refer to an antibody that binds to one or more epitopes or antigenic determinants within lipoteichoic acid, a constituent found on Gram positive bacteria.

As used herein, the term “LTA binding molecule” or “lipoteichoic acid binding molecule” refers to a molecule which specifically binds to one or more epitopes or antigenic determinants within lipoteichoic acid (LTA), a constituent found on Gram positive bacteria. In one embodiment, an LTA binding molecule is a whole antibody. In another embodiment, an LTA binding molecule is an antibody fragment. In one embodiment, an LTA binding molecule is a humanized antibody. In another embodiment, an LTA binding molecule is a human antibody. In another embodiment, an LTA binding molecule is a single chain antibody. In another embodiment, an LTA binding molecule is an immunoconjugate. In another embodiment, an LTA binding molecule is a defucosylated antibody. In yet another embodiment, an LTA binding molecule is a bispecific antibody. In another embodiment, an LTA binding molecule is an aglycosylated antibody.

The term “antibody” or “immunoglobulin” as used interchangeably herein, is intended to refer to proteins comprised of four polypeptide chains, two heavy (H) chains and two light (L) chains inter-connected by disulfide bonds, which has the ability to specifically bind antigen. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each variable region (VH or VL) contains 3 CDRs, designated CDR1, CDR2 and CDR3. Each variable region also contains 4 framework sub-regions, designated FR1, FR2, FR3 and FR4. It is intended that the term “antibody” encompass any Ig class or any Ig subclass (e.g. the IgG1, IgG2, IgG3, and IgG4 subclasses of IgG) obtained from any source (e.g., in exemplary embodiments, humans and non-human primates, and in additional embodiments, mice, rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG₁, IgG₂, IgG₃, and IgG₄) that have been identified in humans and higher mammals.

The term “IgG subclass” refers to the four subclasses of immunoglobulin class IgG-IgG₁, IgG₂, IgG₃, and IgG₄ that have been identified in humans and higher mammals by the γ heavy chains of the immunoglobulins, γ₁-γ₄, respectively.

As used herein, the terms “complementarity determining region” and “CDR” refer to the regions that are primarily responsible for antigen-binding. There are three CDRs in a light chain variable region (LCDR1, LCDR2, and LCDR3), and three CDRs in a heavy chain variable region (HCDR1, HCDR2, and HCDR3). The residues that make up these six CDRs have been characterized by Kabat and Chothia as follows: residues 24-34 (LCDR1), 50-56 (LCDR2) and 89-97 (LCDR3) in the light chain variable region and 31-35 (HCDR1), 50-65 (HCDR2) and 95-102 (HCDR3) in the heavy chain variable region; Kabat et al., (1991) Sequences of Proteins of Immunological Interest, 5th Ed. Public Health Service, National Institutes of Health, Bethesda, Md., herein incorporated by reference; and residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196: 901 917, herein incorporated by reference. Unless otherwise specified, the terms “complementarity determining region” and “CDR” as used herein, include the residues that encompass both the Kabat and Chothia definitions (i.e., residues 24-34 (LCDR1), 50-56 (LCDR2), and 89-97 (LCDR3) in the light chain variable region; and 26-35 (HCDR1), 50-65 (HCDR2), and 95-102 (HCDR3)). Also, unless specified, as used herein, the numbering of CDR residues is according to Kabat.

As used herein, the term “framework” refers to the residues of the variable region other than the CDR residues as defined herein. There are four separate framework sub-regions that make up the framework: FR1, FR2, FR3, and FR4. In order to indicate if the framework sub-region is in the light or heavy chain variable region, an “L” or “H” may be added to the sub-region abbreviation (e.g., “FRL1” indicates framework sub-region 1 of the light chain variable region). Unless specified, the numbering of framework residues is according to Kabat.

As used herein, the term “fully human framework” means a framework with an amino acid sequence found naturally in humans. Examples of fully human frameworks, include, but are not limited to, KOL, NEWM, REI, EU, TUR, TEI, LAY and POM (See, e.g., Kabat et al., (1991) Sequences of Proteins of Immunological Interest, U.S. Department of Health and Human Services, NIH, USA; and Wu et al., (1970) J. Exp. Med. 132, 211 250, both of which are herein incorporated by reference).

As used herein, the term “modify” or “modified amino acid” refers to an amino acid which is different or not the same as the corresponding amino acid residue in the parent A110 heavy chain variable region CDRs or the parent A110 light chain variable region CDRs. For example, if the parent A110 light chain CDR3 amino acid sequence is QQWSSNPPT (SEQ ID NO: 5), one example of a modified amino acid at position 92L would result in a light chain CDR3 sequence of QQWRSNPPT (SEQ ID NO: 18)(the serine residue at position 92L was modified to arginine). A modified amino acid may be any amino acid, including but not limited to, leu, met, ala, val, leu, ile, cys, ser, thr, asp, glu, asn, gln, his, lys, arg, gly, pro, trp, tyr, or phe.

As used herein, the terms “subject” and “patient” refer to any animal, such as a mammal like a dog, cat, bird, livestock, and preferably a human.

The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains). The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

Antibodies can exist in monomeric or polymeric form, for example, IgM antibodies which exist in pentameric form and/or IgA antibodies which exist in monomeric, dimeric or multimeric form.

The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fv, single chains and/or single-chain antibodies. The term “antigen-binding fragment” refers to a polypeptide fragment of an immunoglobulin or antibody that binds antigen or competes with intact antibody (i.e., with the intact antibody from which they were derived) for antigen binding (i.e., specific binding). Antigen-binding fragments can be produced by recombinant DNA techniques, or by enzymatic or chemical cleavage of intact immunoglobulins. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab' fragments. See, e.g., Songsivilai & Lachmann, Clin. Exp. Immunol. 79:315-321 (1990); Kostelny et al., J. Immunol. 148, 1547-1553 (1992).

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant crossreactivity. In exemplary embodiments, the antibody exhibits no crossreactivity (e.g., does not crossreact with non-LTA constituents). “Appreciable” or preferred binding includes binding with an affinity of at least 10⁶, 10⁷, 10⁸, 10⁹ M⁻¹, or 10¹⁰ M⁻¹. Affinities greater than 10⁷M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant crossreactivity” is one that will not appreciably bind to an undesirable entity. Specific binding can be determined according to any art-recognized means for determining such binding. Preferably, specific binding is determined according to Scatchard analysis and/or competitive binding assays. In a further specific embodiment, the binding affinity of the antibodies of the invention is tailored such that the binding affinity to LTA is increased as compared to the binding affinity of known A110 and/or A120 by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.5 fold, at least 3 fold, at least 3.5 fold, at least 4 fold, at least 4.5 fold, at least 5 fold, at least 6 fold, at least 7 fold, at least 8 fold, at least 9 fold, at least 10 fold, at least 15 fold, at least 20 fold, at least 30 fold, at least 40 fold, at least 50 fold, at least 100 fold, at least 200 fold, at least 300 fold at least 400 fold, at least 500 fold, or at least 1000 fold.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The BIACORE™ method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

The dissociation constant, KD, and the association constant, KA, are quantitative measures of affinity. At equilibrium, free antigen (Ag) and free antibody (Ab) are in equilibrium with antigen-antibody complex (Ag-Ab), and the rate constants, ka and kd, quantitate the rates of the individual reactions:

$$Ag + Ab {{⇄}^{ ka }}_{kd} Ag - Ab$$

At equilibrium, ka [Ab][Ag]=kd [Ag−Ab]. The dissociation constant, KD, is given by: KD=kd/ka=[Ag][Ab]/[Ag−Ab]. KD has units of concentration, most typically M, mM, μM, nM, pM, etc. When comparing antibody affinities expressed as KD, having greater affinity for Aβ is indicated by a lower value. The association constant, KA, is given by: KA=KA/KD=[Ag−Ab]/[Ag][Ab]. KA has units of inverse concentration, most typically M⁻¹, mM⁻¹, μM⁻¹, nM⁻¹, pM⁻, etc. As used herein, the term “avidity” refers to the strength of the antigen-antibody bond after formation of reversible complexes.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody. The terms “humanized immunoglobulin” or “humanized antibody” are not intended to encompass chimeric immunoglobulins or antibodies, as defined infra. Although humanized immunoglobulins or antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody. See, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539 (incorporated by reference in their entirety for all purposes).

A “humanized immunoglobulin” or “humanized antibody” of the invention can be made using any of the methods described herein or those that are well known in the art.

The phrase “substantially from a human immunoglobulin or antibody” or “substantially human” means that, when aligned to a human immunoglobulin or antibody amino sequence for comparison purposes, the region shares at least 80-90%, 90-95%, or 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human immunoglobulin or antibody” or “substantially non-human” means having an immunoglobulin or antibody sequence at least 80-95%, preferably at least 90-95%, more preferably, 96%, 97%, 98%, or 99% identical to that of a non-human organism, e.g., a non-human mammal.

The term “significant identity” means that two polypeptide sequences, when optimally aligned, such as by the programs GAP or BESTFIT using default gap weights, share at least 60-70% sequence identity, more preferably at least 70-80% sequence identity, more preferably at least 80-90% identity, even more preferably at least 90-95% identity, and even more preferably at least 95% sequence identity or more (e.g., 99% sequence identity or more). The term “substantial identity” means that two polypeptide sequences, when optimally aligned, such as by the programs GAP or BESTFIT using default gap weights, share at least 80-90% sequence identity, preferably at least 90-95% sequence identity, and more preferably at least 95% sequence identity or more (e.g., 99% sequence identity or more). For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

Preferably, residue positions which are not identical differ by conservative amino acid substitutions. For purposes of classifying amino acids substitutions as conservative or nonconservative, amino acids are grouped as follows: Group I (hydrophobic sidechains): leu, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another class.

Accordingly, another aspect of the invention pertains to CDRs that contain changes in amino acid residues. In one embodiment, such CDRs are at least 70-95%, at least 80-95%, or preferably at least 90-95% identical to the amino acid sequence of a CDR sequence identified herein. In another embodiment, such CDRs are at least 40% identical, 50%, 60%, 70%, 80%, 90%, 91%, 92%, 93%, 94%, 95%, 96%, 97%, 98%, or 99% identical to the amino acid sequence of a CDR sequence identified herein.

Preferably, humanized immunoglobulins or antibodies bind antigen with an affinity that is within a factor of three, four, or five of that of the corresponding non-humanized antibody. For example, if the nonhumanized antibody has a binding affinity of 10⁸ M⁻¹, humanized antibodies will have a binding affinity of at least 3×10⁸ M⁻¹, 4×10⁸ M⁻¹, 5×10⁸ M⁻¹, 3×10⁹M⁻¹, 4×10⁹ M⁻¹, 5×10⁹ M⁻¹. Any numerical value within this range is considered to be a part of this invention, e.g., 3.8, 3.9, 4.1, 4.2, 4.3, 4.4, 4.5 4.6, 4.7, 4.8, 4.9, 5.1, 5.2, 5.3, 5.4, 5.5, 5.6, 5.7, 5.8, 5.9. In a further specific embodiment, the binding affinity of the antibodies of the invention is tailored such that the binding affinity to LTA is increased as compared to the binding affinity of known A110 and/or A120 by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.5 fold, at least 3 fold, at least 3.5 fold, at least 4 fold, at least 4.5 fold, at least 5 fold, at least 6 fold, at least 7 fold, at least 8 fold, at least 9 fold, at least 10 fold, at least 15 fold, at least 20 fold, at least 30 fold, at least 40 fold, at least 50 fold, at least 100 fold, at least 200 fold, at least 300 fold at least 400 fold, at least 500 fold, or at least 1000 fold.

When describing the binding properties of an immunoglobulin or antibody chain, the chain can be described based on its ability to “direct antigen binding”. A chain is said to “direct antigen binding” when it confers upon an intact immunoglobulin or antibody (or antigen binding fragment thereof) a specific binding property or binding affinity. A mutation (e.g., a backmutation) is said to substantially affect the ability of a heavy or light chain to direct antigen binding if it affects (e.g., decreases) the binding affinity of an intact immunoglobulin or antibody (or antigen binding fragment thereof) comprising said chain by at least an order of magnitude compared to that of the antibody (or antigen binding fragment thereof) comprising an equivalent chain lacking said mutation. A mutation “does not substantially affect (e.g., decrease) the ability of a chain to direct antigen binding” if it affects (e.g., decreases) the binding affinity of an intact immunoglobulin or antibody (or antigen binding fragment thereof) comprising said chain by only a factor of two, three, or four of that of the antibody (or antigen binding fragment thereof) comprising an equivalent chain lacking said mutation.

An “antigen” is an entity to which an immunoglobulin or antibody (or antigen-binding fragment thereof) specifically binds.

As used herein, the term “antigen binding site” refers to a site that specifically binds (immunoreacts with) an antigen (e.g., a cell surface or soluble antigen). Antibodies of the invention preferably comprise at least two antigen binding sites. An antigen binding site commonly includes immunoglobulin heavy chain and light chain CDRs and the binding site formed by these CDRs determines the specificity of the antibody. An “antigen binding region” or “antigen binding domain” is a region or domain (e.g., an antibody region or domain that includes an antibody binding site as defined herein).

As used herein, the term “immunotherapy” refers to a treatment, for example, a therapeutic or prophylactic treatment, of a disease or disorder intended to and/or producing an immune response (e.g., an active or passive immune response).

The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

As used herein, the terms “nucleic acid sequence encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage, Therefore, an end of an oligonucleotide or polynucleotide, is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “codon” or “triplet” refers to a group of three adjacent nucleotide monomers which specify one of the naturally occurring amino acids found in polypeptides. The term also includes codons which do not specify any amino acid.

As used herein, the terms “an oligonucleotide having a nucleotide sequence encoding a polypeptide,” “polynucleotide having a nucleotide sequence encoding a polypeptide,” and “nucleic acid sequence encoding a peptide” means a nucleic acid sequence comprising the coding region of a particular polypeptide. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements.

Also, as used herein, there is no size limit or size distinction between the terms “oligonucleotide” and “polynucleotide.” Both terms simply refer to molecules composed of nucleotides. Likewise, there is no size distinction between the terms “peptide” and “polypeptide.” Both terms simply refer to molecules composed of amino acid residues.

As used herein, the terms “complementary” or “complementarity” are used in reference to polynucleotides (i.e., a sequence of nucleotides) related by the base-pairing rules. For example, the sequence “5′-A-G-T 3”, is complementary to the sequence “3-T-C-A-5′”. Complementarity may be “partial”, in which only some of the nucleic acids' bases are matched according to the base pairing rules, or, there may be “complete” or “total” complementarity between the nucleic acids. The degree of complementarity between nucleic acid strands has significant effects on the efficiency and strength of hybridization. This is of particular importance in amplification reactions, as well as in detection methods that depend upon binding between nucleic acids.

As used herein, the term “the complement of” a given sequence is used in reference to the sequence that is completely complementary to the sequence over its entire length. For example, the sequence 5′ A GTA 3′ is “the complement” of the sequence 3′ T C A T 5′. The present invention also provides the complement of the sequences described herein (e.g., the complement of the nucleic acid sequences in SEQ ID NOs: 35, 36, 108, 109, 110, 111, 112, 113, 114 or 115).

The term “homology” (when in reference to nucleic acid sequences) refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous”.

The term “treatment” as used herein, is defined as the application or administration of a therapeutic agent to a patient, or application or administration of a therapeutic agent to an isolated tissue or cell line from a patient, who has an infection or disease, a symptom of infection or disease or a predisposition toward an infection or disease, with the purpose to cure, heal, alleviate, relieve, alter, remedy, ameliorate, improve or affect the disease, the symptoms of infection or disease or the predisposition toward infection or disease.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disease, the patient's general physiology, e.g., the patient's body mass, age, gender, the route of administration, and other factors well known to physicians and/or pharmacologists. Effective doses may be expressed, for example, as the total mass of antibody (e.g., in grams, milligrams or micrograms) or as a ratio of mass of antibody to body mass (e.g., as grams per kilogram (g/kg), milligrams per kilogram (mg/kg), or micrograms per kilogram (μg/kg). An effective dose of antibody used in the present methods will range, for example, between 1 μg/kg and 1 g/kg, preferably between 1 μg/kg and 500 mg/kg. An exemplary range for effective doses of antibodies used in the methods of the present invention is between 0.1 mg/kg and 100 mg/kg. Exemplary effective doses include, but are not limited to, 10 μg/kg, 30 μg/kg, 60 μg/kg, 90 μg/kg, 100 μg/kg, 200 μg/kg, 300 μg/kg, 500 μg/kg, 1 mg/kg, 30 mg/kg, 60 mg/kg, 90 mg/kg, 100 mg/kg, 110 mg/kg, 120 mg/kg, 125 mg/kg, 130 mg/kg, 140 mg/kg, 150 mg/kg, 160 mg/kg, 170 mg/kg, 175 mg/kg, 180 mg/kg, 190 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg and 1 g/kg. In another embodiment, the effective dose can comprise multiple administrations of a dose. For example, the effective dose of 600 mg/kg may consist of the administration of a dose of 100 mg/kg on days 0, 1 and 2, and the administration of a dose of 100 mg/kg weekly thereafter for three weeks. In another embodiment, the effective dose of 600 μg/kg may consist of the administration of a dose of 100 μg/kg on days 0, 1 and 2, and the administration of a dose of 100 μg/kg weekly thereafter for three weeks.

As used herein, the term “administering” refers to the act of introducing a pharmaceutical agent into a patient's body. An exemplary route of administration in the parenteral route, e.g., subcutaneous, intravenous or intraperitoneal administration.

The term “patient” includes human and other mammalian subjects that receive either prophylactic or therapeutic treatment with one or more agents (e.g., immunotherapeutic agents) of the invention. Exemplary patients receive either prophylactic or therapeutic treatment with the immunotherapeutic agents of the invention. In a preferred embodiment, a patient is a neonate.

The terms “animal model” or “model animal,” as used herein, include a member of a mammalian species such as rodents, non-human primates, sheep, dogs, and cows that exhibit features or characteristics of a certain system of disease or disorder, e.g., a human system, disease or disorder, e.g., a bacterial infection. Exemplary non-human animals selected from the rodent family include rabbits, guinea pigs, rats and mice, most preferably mice. An “animal model” of, or “model animal” having, a bacterial infection exhibits, for example, a Staphylococcal bacterial infection.

As used herein, the term “kit” is used in reference to a combination of reagents and other materials which facilitate sample analysis. In some embodiments, the immunoassay kit of the present invention includes a suitable antigen, binding agent comprising a detectable moiety, and detection reagents. A system for amplifying the signal produced by detectable moieties may or may not also be included in the kit. Furthermore, in other embodiments, the kit includes, but is not limited to, components such as apparatus for sample collection, sample tubes, holders, trays, racks, dishes, plates, instructions to the kit user, solutions or other chemical reagents, and samples to be used for standardization, normalization, and/or control samples.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing a methodology of the invention, as described herein. In another embodiment, a “suitable control” or “appropriate control” is a value, level, feature, characteristic, property, etc. determined in a patient, e.g., a control or normal subject exhibiting, for example, normal traits. In yet another embodiment, a “suitable control” or “appropriate control” is a predefined value, level, feature, characteristic, property, etc.

The term “Fc immunoglobulin variant” or “Fc antibody variant” includes immunoglobulins or antibodies (e.g., humanized immunoglobulins, chimeric immunoglobulins, single chain antibodies, antibody fragments, etc.) having an altered Fc region. Fc regions can be altered, for example, such that the immunoglobulin has an altered effector function. In some embodiments, the Fc region includes one or more amino acid alterations in the hinge region, for example, at EU positions 234, 235, 236 and/or 237. Antibodies including hinge region mutations at one or more of amino acid positions 234, 235, 236 and/or 237, can be made, as described in, for example, U.S. Pat. No. 5,624,821, and U.S. Pat. No. 5,648,260, incorporated by reference herein.

The term “effector function” refers to an activity that resides in the Fc region of an antibody (e.g., an IgG antibody) and includes, for example, the ability of the antibody to bind effector molecules such as complement and/or Fc receptors, which can control several immune functions of the antibody such as effector cell activity, lysis, complement-mediated activity, antibody clearance, and antibody half-life.

The term “effector molecule” refers to a molecule that is capable of binding to the Fc region of an antibody (e.g., an IgG antibody) including, but not limited to, a complement protein or a Fc receptor.

The term “effector cell” refers to a cell capable of binding to the Fc portion of an antibody (e.g., an IgG antibody) typically via an Fc receptor expressed on the surface of the effector cell including, but not limited to, lymphocytes, e.g., antigen presenting cells and T cells.

The term “Fc region” refers to a C-terminal region of an IgG antibody, in particular, the C-terminal region of the heavy chain(s) of said IgG antibody. Although the boundaries of the Fc region of an IgG heavy chain can vary slightly, a Fc region is typically defined as spanning from about amino acid residue Cys226 to the carboxyl-terminus of a human IgG heavy chain(s).

The term “aglycosylated” antibody refers to an antibody lacking one or more carbohydrates by virtue of a chemical or enzymatic process, mutation of one or more glycosylation sites, expression in bacteria, etc. An aglycosylated antibody may be a deglycosylated antibody, that is an antibody for which the Fc carbohydrate has been removed, for example, chemically or enzymatically. Alternatively, the aglycosylated antibody may be a nonglycosylated or unglycosylated antibody, that is an antibody that was expressed without Fc carbohydrate, for example by mutation of one or more residues that encode the glycosylation pattern or by expression in an organism that does not attach carbohydrates to proteins, for example bacteria.

“Kabat numbering” unless otherwise stated, is as taught in Kabat et al. (Sequences of Proteins of Immunological Interest, 5th Ed. Public Health Service, National Institutes of Health, Bethesda, Md. (1991)), expressly incorporated herein by reference. “EU numbering” unless otherwise stated, is also taught in Kabat et al. (Sequences of Proteins of Immunological Interest, 5th Ed. Public Health Service, National Institutes of Health, Bethesda, Md. (1991)) and, for example, refers to the numbering of the residues in heavy chain antibody sequences using the EU index as described therein. This numbering system is based on the sequence of the Eu antibody described in Edelman et al., 63(1):78-85 (1969).

The term “Fc receptor” or “FcR” refers to a receptor that binds to the Fc region of an antibody. Typical Fc receptors which bind to an Fc region of an antibody (e.g., an IgG antibody) include, but are not limited to, receptors of the FcγRI, FcγRII, and FcγRIII subclasses, including allelic variants and alternatively spliced forms of these receptors. Other Fc receptors include the neonatal Fc receptors (FcRn) which regulate antibody half-life. Fc receptors are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995).

### II. LTA Antibodies of the Invention

The present invention provides lipoteichoic acid (LTA) antibodies with desirable characteristics. In particular, in some embodiments, the LTA antibodies have a high binding affinity (K_{d}) with regard to LTA. In exemplary embodiments, antibodies bind to LTA with a binding affinity greater than (or equal to) about 10⁶ M⁻¹, 10⁷ M⁻¹, 10⁸ M⁻¹, 10⁹ M⁻¹, or 10¹⁰ M⁻¹ (including affinities intermediate of these values). The present invention is further directed toward nucleic acid sequences which encode said LTA antibodies, and their expression in recombinant host cells. More specifically, the present invention is directed toward LTA binding molecules derived from chimeric A110, which differ by one or more amino acid residues in the CDRs from the A110 parent antibody and specifically bind LTA. A110 is a chimeric IgG1 antibody derived from a murine monoclonal antibody, 96-110. Murine 96-110 is a murine IgG1 antibody, isolated from a mouse immunized with whole S. epidermidis strain Hay (deposited with the American Type Culture Collection (ATCC) on Dec. 19, 1990, under accession number 55133). Its isolation and anti-staphylococcal properties have been described in U.S. Pat. No. 6,610,293, the entire contents of which are herein incorporated by reference. Murine 96-110 was found to specifically bind lipoteichoic acid, a major constituent of the cell wall of Gram positive bacteria. The hybridoma cell line which produces 96-110 was deposited on Jun. 13, 1997, with the ATCC according to the provisions of the Budapest Treaty and was assigned ATCC accession number HB-12368. Another chimeric LTA antibody, A120, is described in U.S. Pat. No. 7,250,494, the entire contents of which are herein incorporated by reference. As described in U.S. Pat. Nos. 6,610,293 and 7,250,494, the A110 and A120 antibodies have been shown to bind and opsonize whole Gram positive bacteria, including multiple strains of S. epidermidis and S. aureus, thereby enhancing phagocytosis and killing of such bacteria in vitro and enhancing protection from lethal infection of such bacteria in vivo.

A110, and the LTA binding molecules of the invention, inhibits the interaction between LTA, a major constituent on the surface of Gram positive bacteria, and its receptor, toll-like receptor 2 (TLR2), on phagocytic cells, e.g., macrophages and neutrophils, which reduces LTA-mediated cytokine production. The antibodies of the invention selectively recognize and bind to all Gram positive bacteria and do not recognize or bind to Gram negative bacteria. The antibodies of the invention are also broadly reactive in that they bind to multiple serotypes of S. epidermidis, S. epidermidis strain Hay, S. hemolyticus, S. hominus and multiple serotypes of S. aureus. In addition to the ability of the antibodies of the invention to block binding of LTA on bacteria to epithelial cells, and hence the subsequent invasion of the bacteria, the antibodies of the invention are also opsonic, thereby enhancing clearance of the bacteria from tissues and blood. The antibodies of the invention, therefore, provide enhanced protection against infection caused by Gram positive bacteria.

Therefore, LTA binding molecules of the present invention possess properties which render them useful for prevention and treatment of Staphylococcal infection wherein modulation of the LTA/TLR2 interaction is desired, including for example, the prevention, reduction or treatment of sepsis in a subject (e.g., human).

As described below in Tables 1-11, the present invention provides numerous CDRs useful for generating LTA binding molecules. The amino acid sequences for the various CDRs which demonstrated improved affinity are depicted below in Tables 1-11 and the Sequence Listing Table, as compared to known affinities of A110 and A120 for LTA.

As shown in Tables 1-11, it has been discovered that mutation of specific CDR residues, e.g., amino acid residues 31L, 92L, 93L, 31H, 52cH, 61H, 98H, 100aH and combinations thereof, results in an increased binding affinity to LTA. Thus, in one embodiment, the antibodies of the invention comprise at least one different, e.g., mutated, amino acid at the specified CDR residues as compared to the corresponding amino acid in A110 or A120, which different amino acid confers an increase in binding affinity to LTA. In certain embodiments, the mutated amino acid is any amino acid. In other embodiments, the mutated amino acid is positively charged.

In still other embodiments, the mutated amino acid at amino acid residue 31L is Arg. In certain embodiments, the mutated amino acid at amino acid residue 92L is Arg. In other embodiments, the mutated amino acid at amino acid residue 93L is Tyr or Lys. In certain embodiments, the mutated amino acid at amino acid residue 31H is Lys. In certain embodiments, the mutated amino acid at amino acid residue 52cH is Lys or Arg. In certain embodiments, the mutated amino acid at amino acid residue 54H is Arg. In certain embodiments, the mutated amino acid at amino acid residue 61H is Pro. In certain embodiments, the mutated amino acid at amino acid residue 58H is Tyr. In certain embodiments, the mutated amino acid at amino acid residue 97H is Gly. In other embodiments, the mutated amino acid at amino acid residue 98H is Lys or Arg. In yet other embodiments, the mutated amino acid at amino acid residue 99H is Glu, Ser or Lys. In further embodiments, the mutated amino acid at amino acid residue 100H is Thr. In still further embodiments, the mutated amino acid at amino acid residue 100aH is His, Asn, Ala or Arg. In certain embodiments, the mutated amino acid at amino acid residue 102H is Lys.

In still other embodiments, the mutated amino acid residue is N31R. In another embodiment, the mutated amino acid residue is S93Y. (Changes from A110 are designated by the A110 amino acid, followed by the position (according to Kabat), and then the new amino acid, e.g., N31R is a change from N at position 31 to R). In another embodiment, the mutated amino acid residue is S93K. In another embodiment, the mutated amino acid residue is S92R. In another embodiment, the mutated amino acid residue is S98R. In another embodiment, the mutated amino acid residue is D100aH. In another embodiment, the mutated amino acid residue is N30K. In another embodiment, the mutated amino acid residue is S52cK. In another embodiment, the mutated amino acid residue is S98R. In another embodiment, the mutated amino acid residue is N54R. In another embodiment, the mutated amino acid residue is S98K. In another embodiment, the mutated amino acid residue is G99S. In another embodiment, the mutated amino acid residue is S52cR. In another embodiment, the mutated amino acid residue is D100aN. In another embodiment, the mutated amino acid residue is G99K. In another embodiment, the mutated amino acid residue is D100aA. In another embodiment, the mutated amino acid residue is D100aR. In another embodiment, the mutated amino acid residue is Y102K. In yet another embodiment, the mutated amino acid residues are S92R and S93K. In yet another embodiment, the mutated amino acid residues are S98R and D100aH.

The present invention contemplates combination of one or more of the novel CDRs shown in Table 1 with a framework sub-region (e.g., an FR1, FR2, FR3, or FR4) in order to generate an LTA binding peptide, or a nucleic acid sequence encoding an LTA binding peptide. In a preferred embodiment, the framework sub-regions (e.g., an FR1, FR2, FR3, or FR4) constitute the parent framework sub-region, e.g., FR1, FR2, FR3, and FR4 from the heavy or light chain of A110 (SEQ ID NOs:37-44). In another embodiment, the framework sub-regions constitute the framework sub-region from the heavy or light chain of A120 (SEQ ID NOs:100-107). Also, the CDRs shown in the table below may be combined, for example, such that three CDRs are present in a light chain variable region, and/or three CDRs are present in a heavy chain variable region.

TABLE 1
CDR Variants

| | | CHANGE(S) | |
|---|---|---|---|
| SEQ ID NO. | CDR | FROM A110* | SEQUENCE |
| SEQ ID NO: 14 | LCDR1 | N31R | RASSSVRYMH |
| SEQ ID NO: 15 | LCDR3 | S93Y | QQWSYNPPT |
| SEQ ID NO: 16 | LCDR3 | S93K | QQWSKNPPT |
| SEQ ID NO: 17 | LCDR3 | S92R, S93K | QQWRKNPPT |
| SEQ ID NO: 18 | LCDR3 | S92R | QQWRSNPPT |
| SEQ ID NO: 19 | HCDR3 | S98R, | RGARGIHYAMDY |
| | | D100aH | |
| SEQ ID NO: 20 | HCDR1 | N30K | GFTFNKYAMN |
| SEQ ID NO: 21 | HCDR2 | S52cK | RIRSKKNNYATFYADSVKD |
| SEQ ID NO: 22 | HCDR3 | S98R | RGARGIDYAMDY |
| SEQ ID NO: 23 | HCDR3 | D100aH | RGASGIHYAMDY |
| SEQ ID NO: 24 | HCDR2 | N54R | RIRSKSNRYATFYADSVKD |
| SEQ ID NO: 25 | HCDR3 | S98K | RGAKGIDYAMDY |
| SEQ ID NO: 26 | HCDR3 | G99S | RGASSIDYAMDY |
| SEQ ID NO: 27 | HCDR2 | S52cR | RIRSKRNNYATFYADSVKD |
| SEQ ID NO: 28 | HCDR3 | D100aN | RGASGINYAMDY |
| SEQ ID NO: 29 | HCDR3 | G99K | RGASKIDYAMDY |
| SEQ ID NO: 30 | HCDR3 | D100aA | RGASGIAYAMDY |
| SEQ ID NO: 31 | HCDR3 | D100aR | RGASGIRYAMDY |
| SEQ ID NO: 32 | HCDR3 | Y102K | RGASGIDYAMDK |

*Changes from A110 are designated by the A110 amino acid, followed by the position (according to Kabat), and then the new amino acid (e.g., N31R is a change from N at position 31 to R).

In particular, the instant invention is based, at least in part, on the discovery of specific positions within each of the A110 CDRs which, when mutated, confer a substantial increase in binding affinity, e.g., greater than 2-fold increase.

As described in more detail below, the instant invention provides the identification of the position where mutations occur within a CDR and result in a substantial increase in binding affinity. For example, residues 31L (Kabat numbering) within the light chain CDR1 has been identified as the position where mutations occur and result in an increase in binding affinity. Residue 27L has also been identified as a mutated position within the light chain CDR1 which results in increased binding affinity. Within the light chain CDR2, residues 52L and 56L have been identified as positions where mutations occur and result in increased binding affinity. Within the light chain CDR3, residues 92L and 93L were identified as two positions where mutations occur and result in increased binding affinity. Residue 31H within the heavy chain CDR1 was identified as the position where mutations occur and result in increased binding affinity, and residues 52cH, 54H and 61H within the heavy chain CDR2 were identified as two positions where mutations occur and result in increased binding affinity. Within the heavy chain CDR3, residue 100aH was identified as the position where mutations occur and result in increased binding affinity. Residue 98H of the heavy chain CDR3 was also identified as another mutated position which results in increased binding affinity. In addition, residues 99H, 100aH and 102H were identified as mutated positions which result in increased binding affinity. Thus, in certain embodiments, the LTA binding molecules of the invention comprise a single mutation in at least one of either the light or heavy chain CDRs, as described herein, which mutation confers an increased binding affinity relative to the parent A110 antibody. In other embodiments, the LTA binding molecules of the invention comprise a combination of mutations in at least one of either the light or heavy chain CDRs, as described herein, which mutations confer an increased binding affinity relative to the parent A110 antibody.

In one embodiment, the LTA binding molecules of the invention comprise a single mutation in at least one of the heavy chain CDRs which mutation confers an increased binding affinity relative to the parent A110 antibody. In certain embodiments, the single mutation occurs in CDR3 of the heavy chain (HCDR3). In preferred embodiments, the mutation is a conservative substitution relative to the parent antibody, A110. In particular embodiments, the mutated amino acid carries a positive charge. In certain preferred embodiments, the mutation occurs at a site selected from the positions set forth in Tables 7-11. In particular embodiments, the mutation occurs at residue 98H (Kabat numbering), 99H, 100aH or 102H, or combinations thereof, in the heavy chain CDR3. In a specific embodiment, amino acid residue 98H in HCDR3 is lysine (K). In a particular embodiment, the LTA binding molecules of the invention comprise the CDRs of antibody mutant B6 (see Tables 7-11). As shown in Example 3, a single mutation, relative to the parent A110 antibody, in mutant B6 confers a 2- to 5-fold increase in binding affinity to LTA relative to the parent. In other embodiments, the mutation occurs at amino acid residue 100aH. In further embodiments, the LTA binding molecules comprise two mutations in HCDR3, e.g., amino acid residue 98H and 100aH. In certain embodiments, amino acid residue 98H in HCDR3 is lysine (K) and amino acid residue 100aH in HCDR3 is histidine (H). In yet another embodiment, the mutation occurs in CDR2 of the heavy chain (HCDR2). In one embodiment, the mutation occurs at residue 52cH (Kabat numbering) in HCDR2. In a specific embodiment, amino acid residue 52cH in HCDR2 is lysine (K). In a particular embodiment, the LTA binding molecules of the invention comprise the CDRs of antibody mutant H2a-B3 (see Table 8).

In other embodiments, the LTA binding molecules of the invention comprise a mutation in HCDR3 in combination with another mutation, i.e., a second mutation, in any one of the CDRs (LCDR1, LCDR2, LCDR3, HCDR1, HCDR2 and/or HCDR3). In particular embodiments, at least one mutation occurs in each of LCDR1, LCDR3, HCDR1, HCDR2 and HCDR3, with an optional mutation in LCDR2. In preferred embodiments, the mutation is a conservative substitution relative to the parent antibody, A110. In particular embodiments, the mutated amino acid carries a positive charge. In certain preferred embodiments, a mutation in HCDR3 is combined with a second mutation, which occurs at residue 31H (Kabat numbering) in HCDR1. In a specific embodiment, amino acid residue 31H in HCDR1 is lysine (K). In a particular embodiment, the LTA binding molecules of the invention comprise the CDRs of antibody mutant G10 (see Tables 7-11). In other embodiments, the second mutation occurs in CDR1 of the light chain (LCDR1). In one embodiment, the second mutation occurs at amino acid residue 31L in LCDR1. In a specific embodiment, amino acid residue 31L in LCDR1 is arginine (R). In a particular embodiment, the LTA binding molecules of the invention comprise the CDRs of antibody mutant D3 (see Tables 7-11).

In other preferred embodiments, the LTA antibodies of the invention comprise an additional mutation in any one of the CDRs (LCDR1, LCDR2, LCDR3, HCDR1, HCDR2 and/or HCDR3) in combination with the first or second mutation as described above. In one embodiment, the further mutation occurs in LCDR3. In a particular embodiment, the mutation occurs at residue 92L of LCDR3. In a specific embodiment, amino acid 92L in LCDR3 is arginine (R). In a particular embodiment, the LTA binding molecules of the invention comprise the CDRs of antibody mutant A1 (see Tables 7-11). In another embodiment, residue 94L of LCDR3 is mutated. In one embodiment, residue 94L of LCDR3 is tyrosine (Y). In certain embodiments, a further mutation occurs in LCDR1. In one embodiment, residue 31L of LCDR1 is mutated. In a specific embodiment, residue 31L of LCDR1 is arginine (R). In a particular embodiment, the LTA binding molecules of the invention comprise the CDRs of antibody mutant C10 (see Tables 7-11).

The invention also contemplates LTA antibodies which comprise the following LCDR consensus sequences: Arg Ala Ser Ser Ser Val Xaa₁ Tyr Met His (LCDR1)(SEQ ID NO: 116); and Gln Gln Trp Xaa₂ Xaa₃ Asn Pro Pro Thr (LCDR3)(SEQ ID NO: 118); wherein Xaa₁, Xaa₂, Xaa₃ are any amino acid, provided that where CDR1 is SEQ ID NO:3 or SEQ ID NO:9, then CDR3 is not SEQ ID NO:5, and where CDR3 is SEQ ID NO:5, then CDR1 is not SEQ ID NO:3 or SEQ ID NO:9. The invention further contemplates LTA antibodies which comprise the following HCDR consensus sequences: Xaa₄ Tyr Ala Met Asn (HCDR1)(SEQ ID NO: 119); Arg Be Arg Ser Lys Xaa₅ Asn Xaa₆ Tyr Ala Thr Xaa₇ Tyr Ala Asp Ser Val Lys Asp (HCDR2)(SEQ ID NO: 120); and Arg Gly Xaa₈ Xaa₉ Xaa₁₀ Xaa₁₁ Xaa₁₂ Tyr Ala Met Asp Xaa₁₃ (HCDR3)(SEQ ID NO: 121), wherein Xaa₄, Xaa₅, Xaa₆, Xaa₇, Xaa₈, Xaa₉, Xaa₁₀, Xaa₁₁, Xaa₁₂, and Xaa₁₃ are any amino acid, provided that i) where CDR1 is SEQ ID NO:6, then CDR2 is not SEQ ID NO:7 and CDR3 is not SEQ ID NO:8; ii) where CDR2 is SEQ ID NO:7 and CDR3 is SEQ ID NO:8, then CDR1 is not SEQ ID NO:6; iii) where CDR1 is SEQ ID NO:11, then CDR3 is not SEQ ID NO:13; and iv) where CDR3 is SEQ ID NO:13, then CDR 1 is not SEQ ID NO:11.

The present invention also provides LTA antibodies comprising one or more VH CDRs and one or more VL CDRs of A1, B6, B7, C10, D3, G10, 1D3, 4B2, 5A11, Com2B8, Com1G2, Com2C7, Com2H1, Com2G4, Com2E1, Com1B4, Com2C2, Com2C5, Com2B11, Com1B12 and Com2A8.

Specifically, the present invention provides LTA antibodies comprising one or more VH CDRs of Com2B8 (SEQ ID NO:48), Com1G2 (SEQ ID NO:50), Com2C7 (SEQ ID NO:52), Com2H1 (SEQ ID NO:54), Com2G4 (SEQ ID NO:56), Com2E1 (SEQ ID NO:58), Com1B4 (SEQ ID NO:60), Com2C2 (SEQ ID NO:62), Com2C5 (SEQ ID NO:64), Com2B11 (SEQ ID NO:66), Com1B12 (SEQ ID NO:68), Com2A8 (SEQ ID NO:70), A1 (SEQ ID NO:72), B6 (SEQ ID NO:74), B7 (SEQ ID NO:76), C10 (SEQ ID NO:78), D3 (SEQ ID NO:80), G10 (SEQ ID NO:82), 1D3 (SEQ ID NO:84), 4B2 (SEQ ID NO:86) and 5A11 (SEQ ID NO:88). In one embodiment, the present invention provides polypeptides comprising one or more VH CDRs of Com2B8 (SEQ ID NO:48), Com1G2 (SEQ ID NO:50), Com2C7 (SEQ ID NO:52), Com2H1 (SEQ ID NO:54), Com2G4 (SEQ ID NO:56), Com2E1 (SEQ ID NO:58), Com1B4 (SEQ ID NO:60), Com2C2 (SEQ ID NO:62), Com2C5 (SEQ ID NO:64), Com2B11 (SEQ ID NO:66), Com1B12 (SEQ ID NO:68), Com2A8 (SEQ ID NO:70), A1 (SEQ ID NO:72), B6 (SEQ ID NO:74), B7 (SEQ ID NO:76), C10 (SEQ ID NO:78), D3 (SEQ ID NO:80), G10 (SEQ ID NO:82), 1D3 (SEQ ID NO:84), 4B2 (SEQ ID NO:86) and 5A11 (SEQ ID NO:88).

The present invention also provides LTA antibodies comprising one or more VL CDRs of Com2B8 (SEQ ID NO:47), Com1G2 (SEQ ID NO:49), Com2C7 (SEQ ID NO:51), Com2H1 (SEQ ID NO:53), Com2G4 (SEQ ID NO:55), Com2E1 (SEQ ID NO:57), Com1B4 (SEQ ID NO:59), Com2C2 (SEQ ID NO:61), Com2C5 (SEQ ID NO:63), Com2B11 (SEQ ID NO:65), Com1B12 (SEQ ID NO:67), Com2A8 (SEQ ID NO:69), A1 (SEQ ID NO:71), B6 (SEQ ID NO:73), B7 (SEQ ID NO:75), C10 (SEQ ID NO:77), D3 (SEQ ID NO:79), G10 (SEQ ID NO:81), 1D3 (SEQ ID NO:83), 4B2 (SEQ ID NO:85) and 5A11 (SEQ ID NO:87). In one embodiment, the invention provides polypeptides comprising one or more VL CDRs of Com2B8 (SEQ ID NO:47), Com1G2 (SEQ ID NO:49), Com2C7 (SEQ ID NO:51), Com2H1 (SEQ ID NO:53), Com2G4 (SEQ ID NO:55), Com2E1 (SEQ ID NO:57), Com1B4 (SEQ ID NO:59), Com2C2 (SEQ ID NO:61), Com2C5 (SEQ ID NO:63), Com2B11 (SEQ ID NO:65), Com1B12 (SEQ ID NO:67), Com2A8 (SEQ ID NO:69), A1 (SEQ ID NO:71), B6 (SEQ ID NO:73), B7 (SEQ ID NO:75), C10 (SEQ ID NO:77), D3 (SEQ ID NO:79), G10 (SEQ ID NO:81), 1D3 (SEQ ID NO:83), 4B2 (SEQ ID NO:85) and 5A11 (SEQ ID NO:87).

The present invention also provides LTA antibodies comprising one or more VH and one or more VL of Com2B8 (SEQ ID NO:47 and SEQ ID NO:48), Com1G2 (SEQ ID NO:49 and SEQ ID NO:50), Com2C7 (SEQ ID NO:51 and SEQ ID NO:52), Com2H1 (SEQ ID NO:53 and SEQ ID NO:54), Com2G4 (SEQ ID NO:55 and SEQ ID NO:56), Com2E1 (SEQ ID NO:57 and SEQ ID NO:58), Com1B4 (SEQ ID NO:59 and SEQ ID NO:60), Com2C2 (SEQ ID NO:61 and SEQ ID NO:62), Com2C5 (SEQ ID NO:63 and SEQ ID NO:64), Com2B11 (SEQ ID NO:65 and SEQ ID NO:66), Com1B12 (SEQ ID NO:67 and SEQ ID NO:68), Com2A8 (SEQ ID NO:69 and SEQ ID NO:70), A1 (SEQ ID NO:71 and SEQ ID NO:72), B6 (SEQ ID NO:73 and SEQ ID NO:74), B7 (SEQ ID NO:75 and SEQ ID NO:76), C10 (SEQ ID NO:77 and SEQ ID NO:78), D3 (SEQ ID NO:79 and SEQ ID NO:80), G10 (SEQ ID NO:81 and SEQ ID NO:82), 1D3 (SEQ ID NO:83 and SEQ ID NO:84), 4B2 (SEQ ID NO:85 and SEQ ID NO:86) and 5A11 (SEQ ID NO:87 and SEQ ID NO:88). In another embodiment, the invention provides polypeptides comprising one or more VH and one or more VL of Com2B8 (SEQ ID NO:47 and SEQ ID NO:48), Com1G2 (SEQ ID NO:49 and SEQ ID NO:50), Com2C7 (SEQ ID NO:51 and SEQ ID NO:52), Com2H1 (SEQ ID NO:53 and SEQ ID NO:54), Com2G4 (SEQ ID NO:55 and SEQ ID NO:56), Com2E1 (SEQ ID NO:57 and SEQ ID NO:58), Com1B4 (SEQ ID NO:59 and SEQ ID NO:60), Com2C2 (SEQ ID NO:61 and SEQ ID NO:62), Com2C5 (SEQ ID NO:63 and SEQ ID NO:64), Com2B11 (SEQ ID NO:65 and SEQ ID NO:66), Com1B12 (SEQ ID NO:67 and SEQ ID NO:68), Com2A8 (SEQ ID NO:69 and SEQ ID NO:70), A1 (SEQ ID NO:71 and SEQ ID NO:72), B6 (SEQ ID NO:73 and SEQ ID NO:74), B7 (SEQ ID NO:75 and SEQ ID NO:76), C10 (SEQ ID NO:77 and SEQ ID NO:78), D3 (SEQ ID NO:79 and SEQ ID NO:80), G10 (SEQ ID NO:81 and SEQ ID NO:82), 1D3 (SEQ ID NO:83 and SEQ ID NO:84), 4B2 (SEQ ID NO:85 and SEQ ID NO:86) and 5A11 (SEQ ID NO:87 and SEQ ID NO:88).

In accordance with the invention disclosed herein, enhanced antibody variants can be generated by combining in a single polypeptide structure one, two or more novel CDR sequences as disclosed herein (see, for example, SEQ ID NOs:3-32 and 89-99).

In a further specific embodiment, the binding affinity of the antibodies of the invention is tailored such that the binding affinity to LTA is increased as compared to the binding affinity of known A110 and/or A120 by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.5 fold, at least 3 fold, at least 3.5 fold, at least 4 fold, at least 4.5 fold, at least 5 fold, at least 6 fold, at least 7 fold, at least 8 fold, at least 9 fold, at least 10 fold, at least 15 fold, at least 20 fold, at least 30 fold, at least 40 fold, at least 50 fold, at least 100 fold, at least 200 fold, at least 300 fold at least 400 fold, at least 500 fold, or at least 1000 fold.

In generating the clones, the basic or reference antibody (light and heavy chain variable regions (CDRs plus Framework) set forth as SEQ ID NOs: 1 and 2, respectively) was used as the “template” for generating the novel CDR sequences of the antibodies of the present invention, the latter imparting improved binding affinity to LTA. Standard approaches to characterizing and synthesizing the six CDR libraries of single mutations were used (see Wu et al, Proc. Natl. Acad. Sci. 95:6037-6042 (1998), the disclosure of which is hereby incorporated by reference in its entirety). Such methods are also described in the following patents and applications: U.S. Pat. Nos. 7,101,978 and 7,175,996, the entire contents of each of which are hereby incorporated by reference.

The present invention also provides sequences that are substantially the same as the CDR sequences (both amino acid and nucleic acid) shown in the above Tables. For example, one or more amino acid may be changed in the sequences shown in the Tables. Also for example, a number of nucleotide bases may be changed in the sequences shown in the Tables. Changes to the amino acid sequence may be generated by changing the nucleic acid sequence encoding the amino acid sequence. A nucleic acid encoding a variant of a given CDR may be prepared by methods known in the art using the guidance of the present specification for particular sequences. These methods include, but are not limited to, preparation by site-directed (or oligonucleotide-mediated) mutagenesis, PCR mutagenesis, and cassette mutagenesis of an earlier prepared nucleic acid encoding the CDR. Site-directed mutagenesis is a preferred method for preparing substitution variants. This technique is well known in the art (see, e.g., Carter et al., (1985) Nucleic Acids Res. 13: 4431 4443 and Kunkel et. al., (1987) Proc. Natl. Acad. Sci. U.S.A 82: 488 492, both of which are hereby incorporated by reference).

Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the starting CDR (see, e.g., Vallette et al., (1989) Nucleic Acids Res. 17: 723 733, hereby incorporated by reference). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., (1985) Gene 34: 315 323, hereby incorporated by reference. The starting material is the plasmid (or other vector) comprising the starting CDR DNA to be mutated. The codon(s) in the starting DNA to be mutated are identified. There should be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a polypeptide variant can be determined, and a nucleic acid sequence encoding such amino acid sequence variant can be generated synthetically. Conservative modifications in the amino acid sequences of the CDRs may also be made. Naturally occurring residues are divided into classes based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic: trp, tyr, phe. Conservative substitutions will entail exchanging a member of one of these classes for another member of the same class. The present invention also provides the complement of the nucleic acid sequences which encode the peptides shown in Tables 1-2, as well as nucleic acid sequences that will hybridize to these nucleic acid sequences under low, medium, and high stringency conditions.

The CDRs of the present invention may be employed with any type of framework, including the parent framework. In one embodiment, CDRs of the present invention are used with framework regions from the parent antibody, A110. In one embodiment, FRL1 is SEQ ID NO:37. In one embodiment, FRL2 is SEQ ID NO:38. In another embodiment, FRL3 is SEQ ID NO:39. In another embodiment, FRL4 is SEQ ID NO:40. In one embodiment, FRH1 is SEQ ID NO:41. In one embodiment, FRH2 is SEQ ID NO:42. In one embodiment, FRH3 is SEQ ID NO:43. In another embodiment, FRH4 is SEQ ID NO:44. In yet another embodiment, CDRs of the present invention are used with framework regions from antibody A120. In one embodiment, FRL1 is SEQ ID NO:100. In one embodiment, FRL2 is SEQ ID NO:101. In one embodiment, FRL3 is SEQ ID NO:102. In one embodiment, FRL4 is SEQ ID NO:103. In one embodiment, FRH1 is SEQ ID NO:104. In one embodiment, FRH2 is SEQ ID NO:105. In one embodiment, FRH3 is SEQ ID NO:106. In one embodiment, FRH4 is SEQ ID NO:107. In another embodiment, the CDRs are used with a mouse framework region. In another embodiment, the CDRs are used with fully human frameworks, or framework sub-regions. In certain embodiments, the frameworks are human germline sequences. Examples of frameworks which can be employed are provided in the NCBI web site which contains the sequences for the currently known human framework regions. Examples of human VH sequences include, but are not limited to, VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, Vh3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81, which are provided in Matsuda et al., (1998) J. Exp. Med. 188:1973 1975, that includes the complete nucleotide sequence of the human immunoglobulin chain variable region locus, herein incorporated by reference. Examples of human VK sequences include, but are not limited to, A1, A10, A11, A14, A17, A18, A19, A2, A20, A23, A26, A27, A3, A30, A5, A7, B2, B3, L1, L10, L1, L12, L14, L15, L16, L18, L19, L2, L20, L22, L23, L24, L25, L4/18a, L5, L6, L8, L9, O1, O11, O12, O14, O18, O2, O4, and O8, which are provided in Kawasaki et al., (2001) Eur. J. Immunol. 31:1017 1028; Schable and Zachau, (1993) Biol. Chem. Hoppe Seyler 374:1001 1022; and Brensing-Kuppers et al., (1997) Gene 191:173 181, all of which are herein incorporated by reference. Examples of human VL sequences include, but are not limited to, V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-4, V4-6, V5-1, V5-2, V5-4, and V5-6, which are provided in Kawasaki et al., (1997) Genome Res. 7:250 261, herein incorporated by reference. Fully human frameworks can be selected from any of these functional germline genes. Generally, these frameworks differ from each other by a limited number of amino acid changes. These frameworks may be used with the CDRs described herein. Additional examples of human frameworks which may be used with the CDRs of the present invention include, but are not limited to, KOL, NEWM, REI, EU, TUR, TEI, LAY and POM (See, e.g., Kabat et al., (1991) Sequences of Proteins of Immunological Interest, U.S. Department of Health and Human Services, NIH, U.S.A; and Wu et al., (1970), J. Exp. Med. 132:211 250, both of which are herein incorporated by reference).

Again, while not necessary to practice or understand the invention, it is believed that the reason the use of germline sequences is expected to help eliminate adverse immune responses in most individuals is as follows. Somatic mutations frequently occur in the variable region of immunoglobulins as a result of the affinity maturation step that takes place during a normal immune response. Although these mutations are predominantly clustered around the hypervariable CDRs, they also impact residues in the framework regions. These framework mutations are not present in the germline genes and are likely to be immunogenic in patients. In contrast, the general population has been exposed to the vast majority of framework sequences expressed from germline genes and, as a result of immunologic tolerance, these germline frameworks are expected to be less, or non-immunogenic in patients. In order to maximize the likelihood of tolerance, genes encoding the variable regions can be selected from a collection of commonly occurring, functional germline genes, and genes encoding VH and VL regions can be further selected to match known associations between specific heavy and light chains of immunoglobulin molecules.

Also within the scope of the invention are humanized antibodies. See, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539 (incorporated by reference in their entirety for all purposes).

The choice of human variable domains, both light and heavy, to be used in making the humanized antibodies is very important to reduce antigenicity. According to the so-called “best fit” method, the sequence of the variable domain of a non-human antibody is compared with the library of known human variable-domain sequences. The human sequence which is closest to that of the non-human parent antibody is then accepted as the human framework for the humanized antibody (Sims et al., J. Immunol. 151: 2296 (1993); Chothia et al., J. Mol. Biol. 196: 901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89: 4285 (1992); Presta et al., J. Immunol. 151: 2623 (1993)).

### III. Generating LTA Antibodies

In preferred embodiments, the LTA binding molecules of the present invention comprise antibodies or antibody fragments (e.g., comprising one or more of the CDRs described herein). An antibody, or antibody fragment, of the present invention can be prepared by recombinant expression of immunoglobulin light and heavy chain genes in a host cell. For example, to express an antibody recombinantly, a host cell may be transfected with one or more recombinant expression vectors carrying DNA fragments encoding the immunoglobulin light and heavy chains of the antibody such that the light and heavy chains are expressed in the host cell and, preferably, secreted into the medium in which the host cell is cultured, from which medium the antibody can be recovered. Standard recombinant DNA methodologies may be used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al., all of which are hereby incorporated by reference.

To express an antibody with one or more of the CDRs of the present invention, DNA fragments encoding the light and heavy chain variable regions are first obtained. These DNAs can be obtained by amplification and modification of known light and heavy chain variable sequences using the polymerase chain reaction (PCR).

Once the VH and VL fragments are obtained, these sequences can be mutated to encode one or more of the CDR amino acid sequences disclosed herein (see, e.g., Tables 1-2). The amino acid sequences encoded by the VH and VL DNA sequences may be compared to the CDRs sequence(s) desired to identify amino acid residues that differ from the sequences. Then the appropriate nucleotides of the DNA sequences are mutated such that the mutated sequence encodes the selected CDRs (e.g., the six CDRs that are selected from Tables 1-2), using the genetic code to determine which nucleotide changes should be made. Mutagenesis of the sequences may be carried out by standard methods, such as PCR-mediated mutagenesis (in which the mutated nucleotides are incorporated into the PCR primers such that the PCR product contains the mutations) or site-directed mutagenesis. In other embodiments, the variable region is synthesized de novo (e.g., using a nucleic acid synthesizer).

Once DNA fragments encoding the desired VH and VL segments are obtained (e.g., by amplification and mutagenesis of VH and VL genes, or synthetic synthesis, as described above), these DNA fragments can be further manipulated by standard recombinant DNA techniques, for example to convert the variable region genes to full-length antibody chain genes, to Fab fragment genes, to a scFv gene, or to other antigen-binding fragments. In these manipulations, a VL- or VH-encoding DNA fragment is operably linked to another DNA fragment encoding another polypeptide, such as an antibody constant region or a flexible linker. The isolated DNA encoding the VH region can be converted to a full-length heavy chain gene by operably linking the VH-encoding DNA to another DNA molecule encoding heavy chain constant regions (CH1, CH2 and CH3). The sequences of human heavy chain constant region genes are known in the art (see e.g., Kabat, E. A., et al., (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be, for example, an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operably linked to another DNA molecule encoding only the heavy chain CH1 constant region.

The isolated DNA encoding the VL region can be converted to a full-length light chain gene (as well as a Fab light chain gene) by operably linking the VL-encoding DNA to another DNA molecule encoding the light chain constant region, CL. The sequences of human light chain constant region genes are known in the art (see e.g., Kabat, E. A., et al., (1991) Sequences of Proteins of immunological Interest, Fifth Edition, U.S. Department of Health and Human Services. NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH-and VL-encoding DNA fragments may be operably linked to another fragment encoding a flexible linker, e.g., encoding the amino acid sequence (Gly₄-Ser)₃(SEQ ID NO: 156), such that the VH and VL sequences can be expressed as a contiguous single-chain protein, with the VL and VH regions joined by the flexible linker (see e.g., Bird et al., (1988) Science 242:423 426; Huston et al., (1988) Proc. Natl. Acad. Sci. U.S.A 85:5879 5883; McCafferty et al., (1990) Nature 348:552 554), all of which are herein incorporated by reference).

To express the antibodies, or antibody fragments of the invention, DNAs encoding partial or full-length light and heavy chains, obtained as described above, may be inserted into expression vectors such that the genes are operably linked to transcriptional and translational control sequences. In this context, the term “operably linked” is intended to mean that an antibody gene is ligated into a vector such that transcriptional and translational control sequences within the vector serve their intended function of regulating the transcription and translation of the antibody gene. The expression vector and expression control sequences are generally chosen to be compatible with the expression host cell used. The antibody light chain gene and the antibody heavy chain gene can be inserted into separate vectors or, more typically, both genes are inserted into the same expression vector. The antibody genes may be inserted into the expression vector by standard methods (e.g., ligation of complementary restriction sites on the antibody gene fragment and vector, or blunt end ligation if no restriction sites are present). Prior to insertion of the light or heavy chain sequences, the expression vector may already carry antibody constant region sequences. For example, one approach to converting the VH and VL sequences to full-length antibody genes is to insert them into expression vectors already encoding heavy chain constant and light chain constant regions, respectively, such that the VH segment is operably linked to the CH segment(s) within the vector and the VL segment is operably linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention may carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990), herein incorporated by reference. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma virus. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., all of which are herein incorporated by reference.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neomycin gene (for G418 selection).

For expression of the light and heavy chains, the expression vector(s) encoding the heavy and light chains may be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAF-dextran transfection and the like.

Appropriate host cells, include for example, bacteria and corresponding bacteriophage expression systems, yeast, avian, insect and mammalian cells. Methods for recombinant expression, screening and purification of populations of altered variable regions or altered variable region polypeptides within such populations in various host systems are well known in the art and are described, for example, in Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1998). The choice of a particular vector and host system for expression and screening of altered variable regions will be known by those skilled in the art and will depend on the preference of the user. Moreover, expression of diverse populations of heteromeric receptors in either soluble or cell surface form using filamentous bacteriophage vector/host systems is well known in the art and is the subject matter of U.S. Pat. No. 5,871,974.

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. U.S.A 77:4216 4220, used with a DHFR selectable marker, e.g., as described in R. J. Kaufman and P. A. Sharp (1982) Mol. Biol. 159:601 621), NSO myeloma cells, COS cells and SP2 cells. When recombinant expression vectors encoding antibody genes are introduced into mammalian host cells, the antibodies are generally produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present invention. For example, it may be desirable to transfect a host cell with DNA encoding either the light chain or the heavy chain of an antibody of this invention. Recombinant DNA technology may also be used to remove some or all of the DNA encoding either or both of the light and heavy chains that is not necessary for binding to LTA. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than LTA by crosslinking an antibody of the invention to a second antibody by standard chemical crosslinking methods.

Also contemplated within the scope of the instant invention are antibody fragments or modified antibodies, discussed in detail below. In one embodiment, fragments of non-human, and/or chimeric antibodies are provided. In another embodiment, fragments of humanized antibodies are provided. Typically, these fragments exhibit specific binding to antigen with an affinity of at least 10⁷, and more typically 10⁸ or 10⁹M⁻¹. Humanized antibody fragments include separate heavy chains, light chains, Fab, Fab′, F(ab′)2, Fabc, and Fv. Fragments are produced by recombinant DNA techniques, or by enzymatic or chemical separation of intact immunoglobulins. The antibody may also be a light chain or heavy chain dimer or any minimal fragment thereof such as a Fv or a single chain construct as described in Ladner et al. U.S. Pat. No. 4,946,778 to Ladner et al., the contents of which is expressly incorporated by reference.

Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis Limited has developed a series of large and highly functional libraries of fully human VH and VL dAbs (more than ten billion different sequences in each library), and uses these libraries to select dAbs that are specific to therapeutic targets. In contrast to many conventional antibodies, Domain Antibodies arc well expressed in bacterial, yeast, and mammalian cell systems. Further details of domain antibodies and methods of production thereof may be obtained by reference to U.S. Pat. No. 6,291,158; 6,582,915; 6,593,081; 6,172,197; 6,696,245; US Serial No. 2004/0110941; European patent application No. 1433846 and European Patents 0368684 &amp; 0616640; WO05/035572, WO04/101790, WO04/081026, WO04/058821, WO04/003019 and WO03/002609, each of which is incorporated herein by reference in its entirety.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts e.g., E. coli (see e.g. U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see e.g., WO 06/079372, which is herein incorporated by reference in its entirety) is a proprietary method for generating Nanobodies against a desired target, based on automated high-throughout selection of B-cells and could be used in the context of the instant invention.

UniBodies are another antibody fragment technology, however this one is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, UniBodies may function to inhibit or silence, but not kill, the cells to which they are bound. Additionally, UniBody binding to cancer cells do not stimulate them to proliferate. Furthermore, because UniBodies are about half the size of traditional IgG4 antibodies, they may show better distribution over larger solid tumors with potentially advantageous efficacy. UniBodies are cleared from the body at a similar rate to whole TgG4 antibodies and are able to bind with a similar affinity for their antigens as whole antibodies. Further details of UniBodies may be obtained by reference to .PCT Publication No. WO2007/059782, which is herein incorporated by reference in its entirety.

Minibodies are dimeric molecules made up of two polypeptide chains each comprising a stabilized scFv molecule (a single polypeptide comprising one or more antigen binding sites, e.g., a VL domain linked by a flexible linker to a VH domain fused to a CH3 domain via a connecting peptide.

Minibodies can be made by constructing an scFv component and connecting peptide-CH3 component using methods described in the art (see, e.g., U.S. Pat. No. 5,837,821 or WO 94/09817A1). These components can be isolated from separate plasmids as restriction fragments and then ligated and recloned into an appropriate vector. Appropriate assembly can be verified by restriction digestion and DNA sequence analysis.

In another embodiment, a tetravalent minibody can be constructed. Tetravalent minibodies can be constructed in the same manner as minibodies, except that two scFv molecules are linked using a flexible linker, e.g., having an amino acid sequence (G₄S)₄G₃AS.

In one embodiment, tetravalent antibodies can be produced by combining a DNA sequence encoding an antibody with a scFv molecule. For example, in one embodiment, these sequences are combined such that the scFv molecule is linked at its N-terminus to the CH3 domain of the antibody via a flexible linker (e.g., a gly/ser linker such as (Gly₄Ser)₃.

In another embodiment a tetravalent antibody can be made by fusing a stabilized scFv molecule to a connecting peptide, which is fused to a CH1 domain to construct a stabilized scFv-Fab tetravalent molecule (Coloma and Morrison, 1997, Nature Biotechnology, 15:159; WO 95/09917).

In some embodiments, the antibodies and antibody fragments of the invention may be chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat conditions that may be alleviated or modulated by administration of the antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In other embodiments of the invention the antibodies or antigen-binding fragments thereof are conjugated to albumen using art recognized techniques.

In another embodiment of the invention, antibodies, or fragments thereof, are modified to reduce or eliminate potential glycosylation sites. Such modified antibodies are often referred to as “aglycosylated” antibodies. In order to improve the binding affinity of an antibody or antigen-binding fragment thereof, glycosylation sites of the antibody can be altered, for example, by mutagenesis (e.g., site-directed mutagenesis). “Glycosylation sites” refer to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. In order to identify potential glycosylation sites within an antibody or antigen-binding fragment, the sequence of the antibody is examined, for example, by using publicly available databases such as the website provided by the Center for Biological Sequence Analysis (see http://www.cbs.dtu.dk/services/NetNGlyc/ for predicting N-linked glycoslyation sites) and http://www.cbs.dtu.dk/services/NetOGlyc/ for predicting O-linked glycoslyation sites). Additional methods for altering glycosylation sites of antibodies are described in U.S. Pat. Nos. 6,350,861 and 5,714,350.

In yet another embodiment of the invention, antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity.

The expressed population of mutated antibodies can be screened for the identification of one or more mutation species exhibiting binding affinity substantially the same or greater than the donor CDR variable region. Screening can be accomplished using various methods well known in the art for determining the binding affinity of a polypeptide or compound. Additionally, methods based on determining the relative affinity of binding molecules to their partner by comparing the amount of binding between the altered variable region polypeptides and the donor CDR variable region can similarly be used for the identification of species exhibiting binding affinity substantially the same or greater than the donor CDR variable region. All of such methods can be performed, for example, in solution or in solid phase. Moreover, various formats of binding assays are well known in the art and include, for example, immobilization to filters such as nylon or nitrocellulose; two-dimensional arrays, enzyme linked immunosorbant assay (ELISA), radioimmune assay (RIA), panning and plasmon resonance. Such methods can be found described in, for example, Sambrook et al., supra, and Ansubel et al.

For the screening of populations of polypeptides such as the mutated variable region populations produced by the methods of the invention, immobilization of the populations of altered variable regions to filters or other solid substrate is particularly advantageous because large numbers of different species can be efficiently screened for antigen binding. Such filter lifts will allow for the identification of altered variable regions that exhibit substantially the same or greater binding affinity compared to the donor CDR variable region. Alternatively, if the populations of altered variable regions are expressed on the surface of a cell or bacteriophage, for example, panning on immobilized antigen can be used to efficiently screen for the relative binding affinity of species within the population and for those which exhibit substantially the same or greater binding affinity than the donor CDR variable region.

Another affinity method for screening populations of altered variable regions polypeptides is a capture lift assay that is useful for identifying a binding molecule having selective affinity for a ligand (Watkins et al., (1997)). This method employs the selective immobilization of altered variable regions to a solid support and then screening of the selectively immobilized altered variable regions for selective binding interactions against the cognate antigen or binding partner. Selective immobilization functions to increase the sensitivity of the binding interaction being measured since initial immobilization of a population of altered variable regions onto a solid support reduces non-specific binding interactions with irrelevant molecules or contaminants which can be present in the reaction.

Another method for screening populations or for measuring the affinity of individual altered variable region polypeptides is through surface plasmon resonance (SPR). This method is based on the phenomenon which occurs when surface plasmon waves are excited at a metal/liquid interface. Light is directed at, and reflected from, the side of the surface not in contact with sample, and SPR causes a reduction in the reflected light intensity at a specific combination of angle and wavelength. Biomolecular binding events cause changes in the refractive index at the surface layer, which are detected as changes in the SPR signal. The binding event can be either binding association or disassociation between a receptor-ligand pair. The changes in refractive index can be measured essentially instantaneously and therefore allows for determination of the individual components of an affinity constant. More specifically, the method enables accurate measurements of association rates (kₒₙ) and disassociation rates (k_{off}). In preferred embodiments of the present invention, the processes disclosed herein produce high potency antibodies having both high affinity and high kₒₙ wherein the affinity constant is at least 10⁹ M⁻¹ and kₒₙ is at least 2.5×10⁵ M⁻¹ s⁻¹, especially where said affinity is at least 10¹⁰ M⁻¹ and said kₒₙ is at least 2.5×10⁵ M⁻¹ S⁻¹, most especially where said affinity constant is at least 10¹¹ M⁻¹ and said kn is at least 2.5×10⁵ M⁻¹ s⁻¹, with most preferred embodiments having very high affinity and kₒₙ, especially where said affinity is at least 10⁹ M⁻¹ and said kₒₙ is at least 5×10⁵ M⁻¹ S⁻¹, and most especially where the affinity constant is at least 10¹⁰ M⁻¹ and kₒₙ is at least 2.5×10⁵ M⁻¹ S⁻¹, a most especially preferred embodiment being one wherein the processes of the invention produce a high potency antibody wherein the affinity constant is at least 10¹¹ M⁻¹ and the kₒₙ is at least 7.5×10⁵ M⁻¹ S⁻¹. It is to be understood that, where high affinity is also sought, any combination of the above mentioned affinity (kₐ) and kinetic association (kₒₙ) values are within the present invention.

Measurements of kₒₙ and k_{off} values can be advantageous because they can identify altered variable regions or optimized variable regions that are therapeutically more efficacious. For example, an altered variable region, or heteromeric binding fragment thereof, can be more efficacious because it has, for example, a higher kₒₙ valued compared to variable regions and heteromeric binding fragments that exhibit similar binding affinity. Increased efficacy is conferred because molecules with higher kₒₙ values can specifically bind and inhibit their target at a faster rate. Similarly, a molecule of the invention can be more efficacious because it exhibits a lower k_{off} value compared to molecules having similar binding affinity. Increased efficacy observed with molecules having lower k_{off} rates can be observed because, once bound, the molecules are slower to dissociate from their target. Although described with reference to the altered variable regions and optimized variable regions of the invention including, heteromeric variable region binding fragments thereof, the methods described above for measuring associating and disassociation rates are applicable to essentially any antibody or fragment thereof for identifying more effective binders for therapeutic or diagnostic purposes.

Methods for measuring the affinity, including association and disassociation rates using surface plasmon resonance are well known in the arts and can be found described in, for example, Jonsson and Malmquist, Advances in Biosensors, 2:291-336 (1992) and Wu et al. Proc. Natl. Acad. Sci. USA, 95:6037-6042 (1998). Moreover, one apparatus well known in the art for measuring binding interactions is a BIAcore 2000 instrument which is commercially available through Pharmacia Biosensor, (Uppsala, Sweden).

Human LTA antibodies are provided by a variety of techniques described below. Some human antibodies are selected by competitive binding experiments, or otherwise, to have the same epitope specificity as a particular mouse antibody, such as one of the mouse monoclonals described herein. Human antibodies can also be screened for a particular epitope specificity by using only a fragment of LTA as the immunogen, and/or by screening antibodies against a collection of deletion mutants of LTA. In one embodiment, human antibodies have human IgG1 isotype specificity.

a. Trioma Methodology

The basic approach and an exemplary cell fusion partner, SPAZ-4, for use in this approach have been described by Oestberg et al., Hybridoma 2:361 (1983); Oestberg, U.S. Pat. No. 4,634,664; and Engleman et al., U.S. Pat. No. 4,634,666 (each of which is incorporated by reference in its entirety for all purposes). The antibody-producing cell lines obtained by this method are called triomas, because they are descended from three cells; two human and one mouse. Initially, a mouse myeloma line is fused with a human B-lymphocyte to obtain a non-antibody-producing xenogeneic hybrid cell, such as the SPAZ-4 cell line described by Oestberg, supra. The xenogeneic cell is then fused with an immunized human B-lymphocyte to obtain an antibody-producing trioma cell line. Triomas have been found to produce antibody more stably than ordinary hybridomas made from human cells.

The immunized B-lymphocytes are obtained from the blood, spleen, lymph nodes or bone marrow of a human donor. If antibodies against a specific antigen or epitope are desired, it is preferable to use that antigen or epitope thereof for immunization. Immunization can be either in vivo or in vitro. For in vivo immunization, B cells are typically isolated from a human immunized with LTA, a fragment thereof, larger polypeptide containing LTA or fragment, or an anti-idiotypic antibody to an antibody to LTA. In some methods, B cells are isolated from the same patient who is ultimately to be administered antibody therapy. For in vitro immunization, B-lymphocytes are typically exposed to antigen for a period of 7-14 days in a media such as RPMI-1640 (see Engleman, supra) supplemented with 10% human plasma.

The immunized B-lymphocytes are fused to a xenogeneic hybrid cell such as SPAZ-4 by well-known methods. For example, the cells are treated with 40-50% polyethylene glycol of MW 1000-4000, at about 37° C., for about 5-10 min. Cells are separated from the fusion mixture and propagated in media selective for the desired hybrids (e.g., HAT or AH). Clones secreting antibodies having the required binding specificity are identified by assaying the trioma culture medium for the ability to bind to LTA or a fragment thereof. Triomas producing human antibodies having the desired specificity are subcloned by the limiting dilution technique and grown in vitro in culture medium. The trioma cell lines obtained are then tested for the ability to bind LTA or a fragment thereof.

Although triomas are genetically stable they do not produce antibodies at very high levels. Expression levels can be increased by cloning antibody genes from the trioma into one or more expression vectors, and transforming the vector into standard mammalian, bacterial or yeast cell lines.

b. Transgenic Non-Human Mammals

Human antibodies against LTA can also be produced from non-human transgenic mammals having transgenes encoding at least a segment of the human immunoglobulin locus. Usually, the endogenous immunoglobulin locus of such transgenic mammals is functionally inactivated. Preferably, the segment of the human immunoglobulin locus includes unrearranged sequences of heavy and light chain components. Both inactivation of endogenous immunoglobulin genes and introduction of exogenous immunoglobulin genes can be achieved by targeted homologous recombination, or by introduction of YAC chromosomes. The transgenic mammals resulting from this process are capable of functionally rearranging the immunoglobulin component sequences, and expressing a repertoire of antibodies of various isotypes encoded by human immunoglobulin genes, without expressing endogenous immunoglobulin genes. The production and properties of mammals having these properties are described in detail by, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148:1547 (1994), Nature Biotechnology 14:826 (1996), Kucherlapati, WO 91/10741 (1991) (each of which is incorporated by reference in its entirety for all purposes). Transgenic mice are particularly suitable. Anti-LTA antibodies are obtained by immunizing a transgenic nonhuman mammal, such as described by Lonberg or Kucherlapati, supra, with LTA or a fragment thereof. Monoclonal antibodies are prepared by, e.g., fusing B-cells from such mammals to suitable myeloma cell lines using conventional Kohler-Milstein technology. Human polyclonal antibodies can also be provided in the form of serum from humans immunized with an immunogenic agent. Optionally, such polyclonal antibodies can be concentrated by affinity purification using LTA or other amyloid peptide as an affinity reagent.

c. Phage Display Methods

A further approach for obtaining human anti-LTA antibodies is to screen a DNA library from human B cells according to the general protocol outlined by Huse et al., Science 246:1275-1281 (1989). As described for trioma methodology, such B cells can be obtained from a human immunized with LTA, fragments, longer polypeptides containing LTA or fragments or anti-idiotypic antibodies. Optionally, such B cells are obtained from a patient who is ultimately to receive antibody treatment. Antibodies binding to LTA or a fragment thereof are selected. Sequences encoding such antibodies (or a binding fragments) are then cloned and amplified. The protocol described by Huse is rendered more efficient in combination with phage-display technology. See, e.g., Dower et al., WO 91/17271, McCafferty et al., WO 92/01047, Herzig et al., U.S. Pat. No. 5,877,218, Winter et al., U.S. Pat. No. 5,871,907, Winter et al., U.S. Pat. No. 5,858,657, Holliger et al., U.S. Pat. No. 5,837,242, Johnson et al., U.S. Pat. No. 5,733,743 and Hoogenboom et al., U.S. Pat. No. 5,565,332 (each of which is incorporated by reference in its entirety for all purposes). In these methods, libraries of phage are produced in which members display different antibodies on their outer surfaces. Antibodies are usually displayed as Fv or Fab fragments. Phage displaying antibodies with a desired specificity are selected by affinity enrichment to an LTA peptide or fragment thereof.

In a variation of the phage-display method, human antibodies having the binding specificity of a selected murine antibody can be produced. See Winter, WO 92/20791. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. A phage showing strong specific binding for LTA (e.g., at least 10⁸ and preferably at least 10⁹ M⁻¹) is selected. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for LTA are selected. These phage display the variable regions of completely human anti-LTA antibodies. These antibodies usually have the same or similar epitope specificity as the murine starting material.

### IV. Uses

The LTA antibodies of the present invention may be used in the prevention and/or treatment of infection caused by Gram positive bacteria, such as coagulase positive and coagulase negative staphylococci, in humans or animals. In particular, the antibodies of the invention may be used in the prevention and/or treatment of sepsis caused by Gram positive bacteria, such as coagulase positive and coagulase negative staphylococci. More particularly, the antibodies of the intention may be used in the prevention of Staphylococcal infections, sepsis, bacteremia, and inflammation in low birth weight neonates, including very low birth weight neonates, e.g., birth weight between 600 and 1300 grams.

In addition to the use of the LTA antibodies of the invention to treat or prevent S. aureus infection as described above, the present invention contemplates the use of these antibodies in a variety of ways, including the detection of the presence of Gram positive bacteria, such as S. aureus, to diagnose a staph infection, whether in a patient or on medical equipment which may also become infected. In accordance with the invention, a preferred method of detecting the presence of staph infections involves the steps of obtaining a sample suspected of being infected by one or more staphylococcal bacteria species or strains, such as a sample taken from an individual, for example, from one's blood, saliva, tissues, bone, muscle, cartilage, or skin. The cells can then be lysed, and the DNA extracted, precipitated and amplified. Following isolation of the sample, diagnostic assays utilizing the antibodies of the present invention may be carried out to detect the presence of S. aureus, and such assay techniques for determining such presence in a sample are well known to those skilled in the art and include methods such as radioimmunoasssay, Western blot analysis and ELISA assays. In general, in accordance with the invention, a method of diagnosing a staphylococcal infection is contemplated wherein a sample suspected of being infected with a staphylococcal infection has added to it an LTA antibody in accordance with the present invention, and staph is indicated by antibody binding to LTA in the sample.

Accordingly, antibodies in accordance with the invention may be used for the specific detection of staphylococcal, for the prevention of infection from staph bacteria, for the treatment of an ongoing infection, or for use as research tools. The term “antibodies” as used herein includes monoclonal, polyclonal, chimeric, single chain, bispecific, simianized, and humanized or primatized antibodies as well as Fab fragments, such as those fragments which maintain the binding specificity of the antibodies to LTA, including the products of an Fab immunoglobulin expression library. Accordingly, the invention contemplates the use of single chains such as the variable heavy and light chains of the antibodies. Generation of any of these types of antibodies or antibody fragments is well known to those skilled in the art. In the present case, optimized chimeric antibodies to LTA have been generated and isolated and shown to have high binding affinity to several strains of live staphylococcal bacteria.

### V. Prophylactic and Therapeutic Methods

The present invention is directed inter alfa to prevention or treatment of infection caused by Gram positive bacteria by administration of antibodies which bind LTA. Preferably, the present invention is directed to the prevention or treatment of Gram positive bacteria in neonates. The invention is also directed to use of the disclosed LTA antibodies in the manufacture of a medicament for the treatment or prevention of infection caused by Gram positive bacteria. Preferably, the invention is directed to the use of the disclosed LTA antibodies in the manufacture of a medicament for the treatment or prevention of infection caused by Gram positive bacteria in neonates.

In one aspect, the invention provides methods of preventing or treating infection caused by Gram positive bacteria. Such Gram positive bacteria include both coagulase positive and coagulase negative Staphylococci. Some methods of the invention comprise administering an effective dosage of an antibody that specifically binds to LTA to the patient. Such methods are particularly useful for preventing or treating infection caused by Gram positive bacteria in human patients. Exemplary methods comprise administering an effective dosage of an antibody that binds to LTA.

Therapeutic antibodies of the invention are typically substantially pure from undesired contaminant. This means that an antibody is typically at least about 50% w/w (weight/weight) pure, as well as being substantially free from interfering proteins and contaminants. Sometimes the antibodies are at least about 80% w/w and, more preferably at least 90 or about 95% w/w pure. However, using conventional protein purification techniques, homogeneous peptides of at least 99% w/w pure can be obtained.

The methods can be used on both asymptomatic patients and those currently showing symptoms of infection. The antibodies used in such methods can be human, humanized, chimeric or nonhuman antibodies, or fragments thereof (e.g., antigen binding fragments) and can be monoclonal or polyclonal, as described herein.

In another aspect, the invention features administering an antibody with a pharmaceutical carrier as a pharmaceutical composition. Alternatively, the antibody can be administered to a patient by administering a polynucleotide encoding at least one antibody chain. The polynucleotide is expressed to produce the antibody chain in the patient. Optionally, the polynucleotide encodes heavy and light chains of the antibody. The polynucleotide is expressed to produce the heavy and light chains in the patient. In exemplary embodiments, the patient is monitored for level of administered antibody in the blood of the patient.

A. Prophylactic and Therapeutic Treatment Regimes and Dosages

In prophylactic applications, pharmaceutical compositions or medicaments are administered to a patient susceptible to, or otherwise at risk of, an infection caused by Gram positive bacteria in an amount sufficient to eliminate or reduce the risk, lessen the severity, or delay the outset of the infection. In therapeutic applications, compositions or medicaments are administered to a patient suspected of, or already suffering from such infection caused by Gram positive bacteria in an amount sufficient to cure, or at least partially arrest, the symptoms of the infection.

An amount adequate to accomplish therapeutic or prophylactic treatment is defined as a therapeutically- or prophylactically-effective dose. In both prophylactic and therapeutic regimes, antibodies are usually administered in several dosages until a sufficient immune response has been achieved. The term “immune response” or “immunological response” includes the development of a humoral (antibody mediated) and/or a cellular (mediated by antigen-specific T cells or their secretion products) response directed against an antigen in a recipient subject. Typically, the immune response is monitored and repeated dosages are given if the immune response starts to wane.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages need to be titrated to optimize safety and efficacy.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. The term “prophylactically effective dose” is defined as an amount sufficient to prevent or protect against disease and its complications in a patient not yet suffering from the disease. Amounts effective for this use will depend upon the severity of the disease, the patient's general physiology. e.g., the patient's body mass, age, gender, the route of administration, and other factors well known to physicians and/or pharmacologists. Effective doses may be expressed, for example, as the total mass of antibody (e.g., in grams, milligrams or micrograms) or as a ratio of mass of antibody to body mass (e.g., as grams per kilogram (g/kg), milligrams per kilogram (mg/kg), or micrograms per kilogram (μg/kg). An effective dose of antibody used in the present methods will range, for example, between 1 μg/kg and 1 g/kg, preferably between 1 μg/kg and 500 mg/kg. An exemplary range for effective doses of antibodies used in the methods of the present invention is between 0.1 mg/kg and 100 mg/kg. Exemplary effective doses include, but are not limited to, 10 μg/kg, 30 μg/kg, 60 μg/kg, 90 μg/kg, 100 μg/kg, 200 μg/kg, 300 μg/kg, 500 μg/kg, 1 mg/kg, 30 mg/kg, 60 mg/kg, 90 mg/kg, 100 mg/kg, 110 mg/kg, 120 mg/kg, 125 mg/kg, 130 mg/kg, 140 mg/kg, 150 mg/kg, 160 mg/kg, 170 mg/kg, 175 mg/kg, 180 mg/kg, 190 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg and 1 g/kg. In another embodiment, the effective dose can comprise multiple administrations of a dose. For example, the effective dose of 600 mg/kg may consist of the administration of a dose of 100 mg/kg on days 0, 1 and 2, and the administration of a dose of 100 mg/kg weekly thereafter for three weeks. In another embodiment, the effective dose of 600 μg/kg may consist of the administration of a dose of 100 μg/kg on days 0, 1 and 2, and the administration of a dose of 100 μg/kg weekly thereafter for three weeks.

Doses intermediate in the above ranges are also intended to be within the scope of the invention. Subjects can be administered such doses hourly, daily, on alternative days, weekly or according to any other schedule determined by empirical analysis.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance, i.e., provide at least some measure of prevention of infection caused by Gram positive bacteria. Such an amount is defined to be a “prophylactic effective dose.” In this use, the precise amounts again depend upon the patient's state of health and general immunity, but generally range from 3 mg/kg to 100 mg/kg per dose. Such prophylactic therapy as described herein may be primary or supplemental to additional treatment, such as antibiotic therapy, for an infection caused by Gram positive bacteria, an infection caused by a different agent, or an unrelated disease.

Doses for nucleic acids encoding antibodies range from about 10 ng to 1 g, 100 ng to 100 mg, 1 μg to 10 mg, or 30-300 μg DNA per patient. Doses for infectious viral vectors vary from 10-100, or more, virions per dose.

Therapeutic antibodies can be administered by parenteral, topical, intravenous, oral, subcutaneous, intraarterial, intracranial, intraperitoneal, intranasal or intramuscular means for prophylactic and/or therapeutic treatment. The most typical route of administration of the antibodies of the invention is intravenous although other routes can be equally effective. For example, the antibodies of the invention can also be administered subcutaneously or via intramuscular injection. Intramuscular injection is most typically performed in the arm or leg muscles. Intramuscular injection or intravenous infusion are preferred for administration of antibody. In some methods, antibodies are administered as a sustained release composition or device, such as a MEDIPAD™ device.

Antibodies of the invention can optionally be administered in combination with other agents that are at least partly effective in treatment of staphylococcal infections, e.g., antibiotics and other anti-bacterial agents. In certain embodiments, an LTA antibody of the invention is administered in combination with a second immunogenic or immunologic agent. For example, an LTA antibody of the invention can be administered in combination with another antibody to LTA. In other embodiments, an LTA antibody of the invention is administered to a patient who has received or is receiving an LTA vaccine. Agents of the invention can also be administered in combination with other agents that enhance access of the therapeutic agent to a target cell or tissue, for example, liposomes and the like. Coadministering such agents can decrease the dosage of a therapeutic antibody or antigen-binding fragment needed to achieve a desired effect.

B. Pharmaceutical Compositions

Antibodies of the invention are often administered as pharmaceutical compositions comprising an active therapeutic antibody, i.e., and a variety of other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, agents of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation, which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249: 1527 (1990) and Hanes, Advanced Drug Delivery Reviews 28:97 (1997)). The agents of this invention can be administered in the form of a depot injection or implant preparation, which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein.

Alternatively, transdermal delivery can be achieved using a skin patch or using transferosomes (Paul et al., Eur. J. Immunol. 25:3521 (1995); Cevc et al., Biochem. Biophys. Acta 1368:201-15 (1998)).

C. Kits

The invention further provides kits which may be useful in isolating and identifying staphylococcal bacteria and infection. Typically, such kits comprise the LTA antibodies of the present invention in a suitable form, such as lyophilized in a single vessel which then becomes active by addition of an aqueous sample suspected of containing the staphylococcal bacteria. Such a kit will typically include a suitable container for housing the antibodies in a suitable form along with a suitable immunodetection reagent which will allow identification of complexes binding to the LTA antibodies of the invention. For example, the immunodetection reagent may comprise a suitable detectable signal or label, such as a biotin or enzyme that produces a detectable color, etc., which normally may be linked to the antibody or which can be utilized in other suitable ways so as to provide a detectable result when the antibody binds to the antigen.

Kits also typically contain labeling providing directions for use of the kit. The labeling may also include a chart or other correspondence regime correlating levels of measured label with levels of LTA antibodies. The term labeling refers to any written or recorded material that is attached to, or otherwise accompanies a kit at any time during its manufacture, transport, sale or use. For example, the term labeling encompasses advertising leaflets and brochures, packaging materials, instructions, audio or videocassettes, computer discs, as well as writing imprinted directly on kits.

Practice of the present invention will employ, unless indicated otherwise, conventional techniques of cell biology, cell culture, molecular biology, microbiology, recombinant DNA, protein chemistry, and immunology, which are within the skill of the art. Such techniques are described in the literature. See, for example, Molecular Cloning: A Laboratory Manual, 2nd edition. (Sambrook, Fritsch and Maniatis, eds.), Cold Spring Harbor Laboratory Press, 1989; DNA Cloning, Volumes I and II (D. N. Glover, ed), 1985; Oligonucleotide Synthesis, (M. J. Gait, ed.), 1984; U.S. Pat. No. 4,683,195 (Mullis et al.,); Nucleic Acid Hybridization (B. D. Hames and S. J. Higgins, eds.), 1984; Transcription and Translation (B. D. Hames and S. J. Higgins, eds.), 1984; Culture of Animal Cells (R. I. Freshney, ed). Alan R. Liss, Inc., 1987; Immobilized Cells and Enzymes, IRL Press, 1986; A Practical Guide to Molecular Cloning (B. Perbal), 1984; Methods in Enzymology, Volumes 154 and 155 (Wu et al., eds), Academic Press, New York; Gene Transfer Vectors for Mammalian Cells (J. H. Miller and M. P. Calos, eds.), 1987, Cold Spring Harbor Laboratory; Immunochemical Methods in Cell and Molecular Biology (Mayer and Walker, eds.), Academic Press, London, 1987; Handbook of Experiment Immunology, Volumes I-IV (D. M. Weir and C. C. Blackwell, eds.), 1986; Manipulating the Mouse Embryo, Cold Spring Harbor Laboratory Press, 1986.

The following Examples are provided to illustrate the present invention, and should not be construed as limiting thereof.

### Examples

Throughout the examples, the following materials and methods were used unless otherwise stated.

### Materials and Methods

Opsonization Assays

An opsonization assay can be a calorimetric assay, a chemiluminescent assay, a fluorescent or radiolabel uptake assay, a cell-mediated bactericidal assay, or any other appropriate assay known in the art which measures the opsonic potential of a substance and identifies broadly reactive immunoglobulin. In an opsonization assay, the following are incubated together: an infectious agent, a eukaryotic cell, and the opsonizing substance to be tested, or an opsonizing substance plus a purported opsonizing enhancing substance. Preferably, the opsonization assay is a cell-mediated bactericidal assay. In this in vitro assay, the following are incubated together: an infectious agent, typically a bacterium, a phagocytic cell, and an opsonizing substance, such as immunoglobulin. Although any eukaryotic cell with phagocytic or binding ability may be used in a cell-mediated bactericidal assay, a macrophage, a monocyte, a neutrophil, or any combination of these cells, is preferred. Complement proteins may be included to promote opsonization by both the classical and alternate pathways.

The opsonic ability of immunoglobulin is determined from the amount or number of infectious agents remaining after incubation. In a cell-mediated bactericidal assay, this is accomplished by comparing the number of surviving bacteria between two similar assays, only one of which contains the purported opsonizing immunoglobulin. Alternatively, the opsonic ability is determined by measuring the numbers of viable organisms before and after incubation. A reduced number of bacteria after incubation in the presence of immunoglobulin indicates a positive opsonizing ability. In the cell-mediated bactericidal assay, positive opsonization is determined by culturing the incubation mixture under appropriate bacterial growth conditions. Any significant reduction in the number of viable bacteria comparing pre- and post-incubation samples, or between samples which contain immunoglobulin and those that do not, is a positive reaction.

Clearance/Protective Assays

A clearance/protective assay may be used to measure clearance and protection. A particularly useful animal model comprises administering an antibody and a Gram positive organism to an immunocompromised (e.g., an immature) animal, followed by evaluating whether the antibody reduces mortality of the animal or enhances clearance of the organism from the animal. This assay may use any immature animal, including the rabbit, the guinea pig, the mouse, the rat, or any other suitable laboratory animal. The suckling rat lethal animal model, as described in Fischer et al. (1994), Weisman et al. (1993), Fischer et al., (1992) and Fischer et al. (1986) is most preferred (see, e.g., Fischer et al. (1994) J. Infect. Dis., 169(2):324-329; Weisman et al. (1993) J. Pediatr., 122(6):929-937; Fischer et al. (1992) Clin. Immunol. Immunopathol., 62(1 Pt 2):S92-S97; and Fischer et al. (1986) Pediatr. Infect. Dis., 5(3 Suppl):S171-S175, the entire contents of each of which are incorporated herein by reference). Such a model can readily incorporate an infected foreign body, such as an infected catheter, to more closely mimic the clinical setting. An alternative model utilizes adult susceptible animals, such as CF1 mice.

Clearance is evaluated by determining whether the pharmaceutical composition enhances clearance of the infectious agent from the animal. This is typically determined from a sample of biological fluid, such as blood, peritoneal fluid, or cerebrospinal fluid. The infectious agent is cultured from the biological fluid in a manner suitable for growth or identification of the surviving infectious agent. From samples of fluid taken over a period of time after treatment, one skilled in the art can determine the effect of the pharmaceutical composition on the ability of the animal to clear the infectious agent. Further data may be obtained by measuring over a period of time, preferably a period of days, survival of animals to which the pharmaceutical composition is administered. Typically, both sets of data are utilized. Results are considered positive if the pharmaceutical composition enhances clearance or decreases mortality. In situations in which there is enhanced organism clearance, but the test animals still perish, a positive result is still indicated.

### Example 1

### Construction of Variants

A number of variants derived from the parent A110 chimeric antibody, an anti-lipoteichoic acid (LTA) antibody, were generated by Applied Molecular Evolution (San Diego, Calif.) using their codon based mutagenesis and antibody engineering technology as described in Huse et al. (Huse et al., (1993) Intern. Rev. Immunol. 10:129-137, the entire contents of which are incorporated herein by reference) and Wu et al. (Wu et al., (1998) PNAS 95:6037-42, the entire contents of which are incorporated herein by reference). The nucleic acid sequences encoding the A110 CDRs and framework regions comprising the light and heavy chains are shown in SEQ ID NOs: 33 and 34, respectively. The six A110 CDRs employed are as follows: CDRL1 (SEQ ID NO:3); CDRL2 (SEQ ID NO: 4); CDRL3 (SEQ ID NO:5); CDRH1 (SEQ ID NO:6); CDRH2 (SEQ ID NO:7); and CDRH3 (SEQ ID NO8).

| | |
|---|---|
| SEQ ID NO: 3 | RASSSVNYMH |
| SEQ ID NO: 4 | ATSNLAS |
| SEQ ID NO: 5 | QQWSSNPPT |
| SEQ ID NO: 6 | GFTFNNYAMN |
| SEQ ID NO: 7 | RIRSKSNNYATFYADSVKD |
| SEQ ID NO: 8 | RGASGIDYAMDY |

Affinity optimization of the parent A110 antibody was performed using codon-based mutagenesis of each CDR region. In order to identify mutations that improve the binding properties, variable regions of A110 light chain and heavy chain genes were cloned into bacteriophage M13 expression vectors. CDR regions were individually deleted by oligonucleotide-directed mutagenesis as described by Kunkel (Kunkel, (1985) Proc. Natl. Acad. Sci. U.S.A, 82: 488-492, the entire contents of which are incorporated herein by reference) to create mutagenesis templates. Codon based mutagenesis for oligonucleotide synthesis to yield CDR sequences of the invention was employed. Seven variant libraries were constructed by Kunkel mutagenesis using corresponding CDR-deletion template and a pool of mutagenic oligonucleotides. In this example, the CDRs include residues that encompass both the Kabat and Chothia definitions (e.g., residues 26-35 for CDRH1). The length of CDRH2 made it necessary to construct two separate libraries to cover the entire region.

The mutant light chain libraries were screened with filter lift assay as described in Watkins et al. (1997) Anal. Biochem. 253:37-45, the entire contents of which are incorporated herein by reference. The heavy chain libraries were characterized by sequencing 20 random picked clones.

Characterization of the CDR mutation libraries, including number of variants and mutation efficiency, is provided in Table 2 below.

TABLE 2
Characterization of the CDR Mutation Libraries

| | Length of CDR | | Mutation |
|---|---|---|---|
| Libraries | (amino acid) | No. of variants | Efficiency (%) |
| H1 | 5 | 5 × 32 = 160 | 80 (16/20) |
| H2a | 10 | 10 × 32 = 320 | 70 (14/20) |
| H2b | 9 | 9 × 32 = 288 | 65 (13/20) |
| H3 | 12 | 12 × 32 = 384 | 75 (15/20) |
| L1 | 10 | 10 × 32 = 320 | 61.0 (83/163) |
| L2 | 7 | 7 × 32 = 224 | 68.4 (141/206) |
| L3 | 9 | 9 × 32 = 288 | 76.8 (192/250) |

The mutation distribution across the CDRs is shown below in Table 3. As shown in Tables 3a-3g, mutations are randomly distributed along the CDRs

### Table 3. Mutation Distribution

TABLE 3a
LCDR1

| | Kabat No. | | | | | | | | | | |
|---|---|---|---|---|---|---|---|---|---|---|---|
| | 24 | 25 | 26 | 27 | 28 | 29 | 31 | 32 | 33 | 34 | SEQ ID NO |
| Hu96-110 | R | A | S | S | S | V | N | Y | M | H | 3 |
| Mutation | | C | P | E | A | | I | N | L | P | 125 |
| | | G | | V | L | | T | T | K | | 126 |
| | | L | | A | | | | M | | | 127 |
| Total | | 3 | 2 | 3 | 2 | | 2 | 3 | 2 | 1 | |
| No. | | | | | | | | | | | |

TABLE 3b
LCDR2

| | Kabat No. | | | | | | | |
|---|---|---|---|---|---|---|---|---|
| | 50 | 51 | 52 | 53 | 54 | 55 | 56 | SEQ ID NO |
| Hu96-110 | A | T | S | N | L | A | S | 4 |
| Mutation | P | K | T | R | T | T | P | 128 |
| | T | S | D | P | A | | | 129 |
| | | A | | G | S | | | 130 |
| | | | | E | | | | 131 |
| | | | | T | | | | 132 |
| Total No. | 2 | 3 | 2 | 5 | 3 | 1 | 2 | |

TABLE 3c
LCDR3

| | Kabat No. | | | | | | | | | |
|---|---|---|---|---|---|---|---|---|---|---|
| | 89 | 90 | 91 | 92 | 93 | 94 | 95 | 96 | 97 | SEQ ID NO |
| Hu96-110 | Q | Q | W | S | S | N | P | P | T | 5 |
| Mutation | P | R | R | A | P | L | | (P) | | 133 |
| | | A | | V | F | P | | | | 134 |
| | | T | | | | T | | | | 135 |
| | | P | | | | | | | | 136 |
| Total No. | 2 | 4 | 1 | 2 | 2 | 5 | | 2 | | |

TABLE 3d
HCDR1

| | Kabat No. | | | | | |
|---|---|---|---|---|---|---|
| | 31 | 32 | 33 | 34 | 35 | SEQ ID NO |
| Hu96-110 | N | Y | A | M | N | 137 |
| Mutation | H | E | E | S | Q | 138 |
| | R | K | R | H | | 139 |
| | W | R | | I | | 140 |
| | A | V | | W | | 141 |
| | | | | K | | 142 |
| Total No. | 4 | 4 | 2 | 6 | 1 | |

TABLE 3e
HCDR2a

| | Kabat No. | | | | | | | | | | |
|---|---|---|---|---|---|---|---|---|---|---|---|
| | 50 | 51 | 52 | 52a | 52b | 52c | 53 | 54 | 55 | 56 | SEQ ID NO |
| Hu96-110 | R | I | R | S | K | S | N | N | Y | A | 143 |
| Mutation | Y | S | S | K | ? | | T | T | | | 144 |
| | H | ? | | | | | R | Y | | | 145 |
| | | | | | | | | S | | | 146 |
| Total | 3 | 2 | 1 | 1 | 1 | | 2 | 4 | | | |
| No. | | | | | | | | | | | |

TABLE 3f
HCDR2b

| | Kabat No. | | | | | | | | | |
|---|---|---|---|---|---|---|---|---|---|---|
| | 57 | 58 | 59 | 60 | 61 | 62 | 63 | 64 | 65 | SEQ ID NO |
| Hu96-110 | T | F | Y | A | D | S | V | K | D | 147 |
| Mutation | S | R | L | T | P | | P | F | | 148 |
| | R | H | | K | K | | R | | | 149 |
| Total No. | 2 | 3 | 1 | 2 | 2 | | 2 | 1 | | |

TABLE 3g

HCDR3

Kabat No.

| | | | | | | | | | | | | |
|---|---|---|---|---|---|---|---|---|---|---|---|---|
| | | | | | | | | | | | | SEQ ID |
| 95 | 96 | 97 | 98 | 99 | 100 | 100a | 100b | 100c | 100d | 101 | 102 | NO |

| | | | | | | | | | | | | | |
|---|---|---|---|---|---|---|---|---|---|---|---|---|---|
| Hu96-110 | R | G | A | S | G | I | D | Y | A | M | D | Y | 8 |
| Mutation | | | T | | S | H | K | K | H | K | A | M | 150 |
| | | | | | | | | | C | H | | | 151 |
| | | | | | | | | | | N | | | 152 |
| | | | | | | | | | | L | | | 153 |
| Total No. | | | 1 | | 2 | 2 | 1 | 1 | 2 | 4 | 1 | 1 | |

### Example 2

### Screening of CDR Libraries

CDR mutant libraries were initially screened by capture lift to identify the highest affinity variants for binding to biotinylated lipoteichoic acid (Biotin-LTA). The Biotin-LTA was prepared freshly using Biotin LC hydrazide (Pierce Cat #21340), which is stable for up to two weeks if stored at 4° C. The capture lift procedure was performed as described previously (see, e.g., Huse et al. (1992) J. Immunol. 149:3914-3920; Watkins et al. (1997) Anal. Biochem. 253:37-45; Watkins et al. (2002) Methods Mol. Biol. 178:187-93; WO/0164751; and US2002/0098189, the entire contents of each of which are incorporated herein by reference).

Subsequently, desired clones were characterized for antigen binding by single-point ELISA (SPE) (see, e.g., Watkins et al., supra, 1997) and titration of Fab proteins on immobilized LTA in an ELISA format. Following such screening, clones of interest were sequenced and mutations that enhance antigen-binding activity were identified. Affinity-enhanced clones with unique mutations were further characterized by live bacteria binding (LBA) assay using a panel of S. epidermidis strains.

A summary of the results from the CDR library screens are shown in Tables 4-9 below.

TABLE 4
Beneficial Mutations in LCDR1

| | Kabat No. | | | | | | | | | | | SEQ |
|---|---|---|---|---|---|---|---|---|---|---|---|---|
| | | | | | | | | | | | | ID |
| | 24 | 25 | 26 | 27 | 28 | 29 | 31 | 32 | 33 | 34 | OD₅₆₀ | NO: |
| Hu96-110 | R | A | S | S | S | V | N | Y | M | H | | 3 |
| IX- | | | | | | | | | | | 0.555 | 3 |
| 110his | | | | | | | | | | | | |
| L1-A4 | | | | R | | | | | | | 0.819 | 89 |
| L1-C10 | | | | | | | R | | | | 0.801 | 14 |
| L1-E1 | | | | | | | K | | | | 0.731 | 90 |

TABLE 5
Beneficial Mutations in LCDR2

| | Kabat No. | | | | | | | | |
|---|---|---|---|---|---|---|---|---|---|
| | 50 | 51 | 52 | 53 | 54 | 55 | 56 | OD₅₆₀ | SEQ ID NO: |
| Hu96-110 | A | T | S | N | L | A | S | | 4 |
| IX-110his | | | | | | | | 0.555 | 4 |
| L2-A2 | | | L | | | | | 0.620 | 91 |
| L2-A4 | | | I | | | | | 0.666 | 92 |

TABLE 6
Beneficial Mutations in LCDR3

| | | | | | | | | | | | SEQ |
|---|---|---|---|---|---|---|---|---|---|---|---|
| | Kabat No. | | | | | | | | | | ID |
| | 89 | 90 | 91 | 92 | 93 | 94 | 95 | 96 | 97 | OD₅₆₀ | NO: |
| Hu96-110 | Q | Q | W | S | S | N | P | P | T | | 5 |
| IX-110his | | | | | | | | | | 0.555 | 5 |
| L3-A1 | | | | R | | | | | | 0.679 | 93 |
| L3-B2 | | | | | K | | | | | 0.844 | 16 |
| L3-C10 | | | | | Y | | | | | 0.606 | 15 |
| L3-3C9 | | | | | R | | | | | 0.739 | 94 |

TABLE 7
Beneficial Mutations in HCDR1

| | | | | | | | | | | | | SEQ |
|---|---|---|---|---|---|---|---|---|---|---|---|---|
| | Kabat No. | | | | | | | | | | | ID |
| | 26 | 27 | 28 | 29 | 30 | 31 | 32 | 33 | 34 | 35 | OD₅₆₀ | NO: |
| Hu96-110 | G | F | T | F | N | N | Y | A | M | N | | 6 |
| IX- | | | | | | | | | | | 0.555 | 6 |
| 110his | | | | | | | | | | | | |
| H1-B3 | | | | | | K | | | | | 0.647 | 20 |
| H1-C3 | | | | | | R | | | | | 0.668 | 95 |

TABLE 8

Beneficial Mutations in HCDR2

| | |
|---|---|
| Kabat No. | SEQ ID |

| | | | | | | | | | | | | | | | | | | | | |
|---|---|---|---|---|---|---|---|---|---|---|---|---|---|---|---|---|---|---|---|---|
| 50 | 51 | 52 | 52a | 52b | 52c | 53 | 54 | 55 | 56 | 57 | 58 | 59 | 60 | 61 | 62 | 63 | 64 | 65 | OD₅₆₀ | NO: |

| | | | | | | | | | | | | | | | | | | | | | |
|---|---|---|---|---|---|---|---|---|---|---|---|---|---|---|---|---|---|---|---|---|---|
| Hu96-110 | R | I | R | S | K | S | N | N | Y | A | T | F | Y | A | D | S | V | K | D | | 7 |
| IX-110his | | | | | | | | | | | | | | | | | | | | 0.555 | 7 |
| H2a-A1 | | | | | | | | R | | | | | | | | | | | | 0.657 | 24 |
| H2a-C10 | | | | | | R | | | | | | | | | | | | | | 0.793 | 27 |
| H2a-B3 | | | | | | K | | | | | | | | | | | | | | 0.885 | 21 |
| H2a-C9 | | | | | | | | K | | | | | | | | | | | | 0.650 | 96 |
| H2b-A4 | | | | | | | | | | | | | | | P | | | | | 0.719 | 97 |

TABLE 9

Beneficial Mutations in HCDR3

| | |
|---|---|
| Kabat No. | SEQ ID |

| | | | | | | | | | | | | | |
|---|---|---|---|---|---|---|---|---|---|---|---|---|---|
| 95 | 96 | 97 | 98 | 99 | 100 | 100a | 100b | 100c | 100d | 101 | 102 | OD₅₆₀ | NO: |

| | | | | | | | | | | | | | | |
|---|---|---|---|---|---|---|---|---|---|---|---|---|---|---|
| Hu96-110 | R | G | A | S | G | I | D | Y | A | M | D | Y | | 8 |
| IX-110his | | | | | | | | | | | | | 0.555 | 8 |
| H3-A1 | | | | R | | | | | | | | | 0.857 | 22 |
| H3-B6 | | | | K | | | | | | | | | 0.846 | 25 |
| H3-B7 | | | | | S | | | | | | | | 0.598 | 26 |
| H3-C10 | | | | | | | H | | | | | | 0.747 | 98 |
| H3-D3 | | | | | | | N | | | | | | 0.752 | 99 |
| H3-G10 | | | | | K | | | | | | | | 0.780 | 29 |
| H3-1D3 | | | | | | | A | | | | | | 0.572 | 30 |
| H3-4B2 | | | | | | | R | | | | | | 0.673 | 31 |
| H3-5A11 | | | | | | | | | | | | K | 0.637 | 32 |

Eighty-five selected hits were sequenced. There were 25 unique mutants with enhanced activity as shown in Tables 4-9. There were 28 unique mutants with at least 10% higher activity. Many of them were found multiple times. Mutations were found in all six CDRs, but were mainly located at thirteen positions. Most beneficial mutations were positive-charged residues (R or K; there were 49 Rs or Ks out of 78 mutations).

### Example 3

### Characterization of HCDR3 Beneficial Variants

Characterizations were carried out for all unique CDR mutants. The following example details characterization of the HCDR3 variants. However, this method was used to characterize all six CDRs.

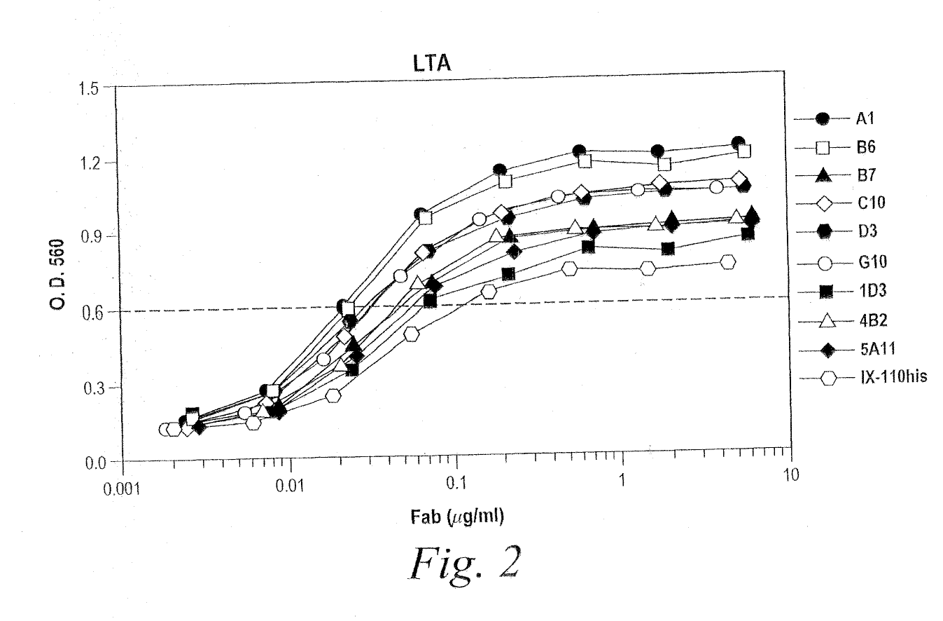

Nine HCDR3 mutants (A1, B6, B7, C10, D3, G10, 1D3, 4B2 and 5A11), were characterized by Fab titration for binding to biotinylated-LTA (b-LTA). FIG. 2 depicts the results of an LTA binding assay of the HCDR3 beneficial variants in anti-Fab capture format. The LTA binding assay was performed essentially as previously described. Briefly, individual Fab fragments were produced in E. coli and periplasmic extracts were tested in ELISA for binding to biotinylated LTA (b-LTA). In order to identify LTA-binding Fab fragments, Fab fragments were captured from periplasmic extracts to an ELISA plate using an anti-Fab antibody, biotinylated-LTA (b-LTA) added and detected with NeutrAvidin™ (Pierce) conjugated to alkaline phosphatase (NeutrAvidin™-AP). A serial 3-fold titration of periplasmic Fab from a 15 ml culture was added to wells which were coated, 50 μl/well, with 2 μg/ml anti-human Fab antibody. Next, 50 μl/well of 1 μg/ml biotinylated LTA in PBSt was added and detected with NeutrAvidin™-AP. The ELISA results are shown in FIG. 2. As shown in FIG. 2, all of the mutants showed improved affinity as compared to IX-110his. Five mutants (A1, B6, C10, D3 and G10) exhibited 2-5-fold higher affinity. This data also correlated well with data obtained form the filter lift assay and Single Point Elisa (SPE).

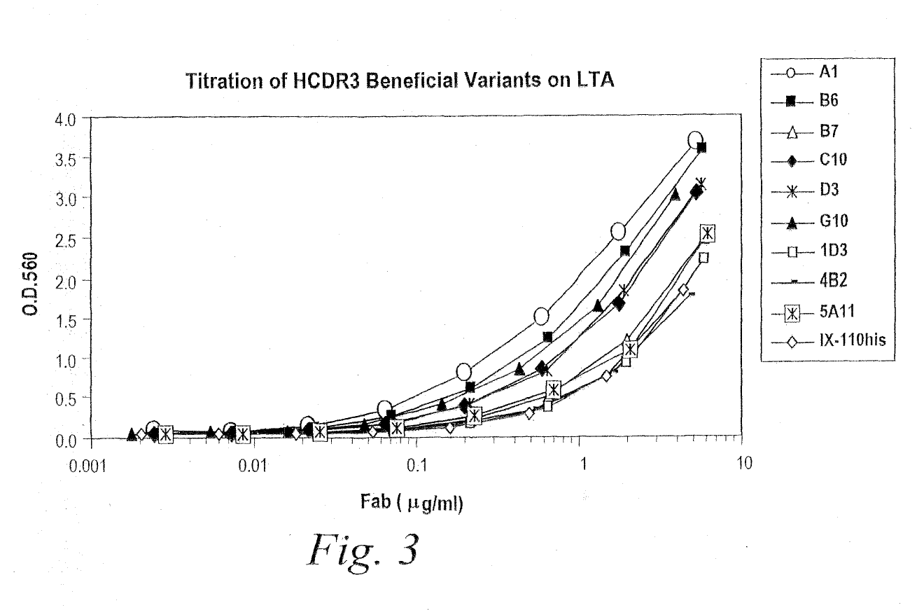

The nine HCDR3 mutants were also characterized by a direct LTA assay. FIG. 3 depicts the titration of the HCDR3 beneficial variants on LTA. As shown in FIG. 3, the direct LTA assay showed the same profile of activity improvement as in the Fab-capture assay, described above (see FIG. 2). The results from the two assays, the direct LTA assay and Fab-capture assay, show that the selected mutants enhanced binding activity specifically to LTA, not to biotin. The results also show that titration is not saturated at about 5 μg/ml Fab in this assay format. Importantly, papain Fab did not show saturation in this assay at a concentration of 50 μg/ml.

### Example 4

### Titration of HCDR3 Beneficial Clones on Live Bacteria

Select HCDR3 beneficial clones (A1, C10, D3, G10), identified as having superior binding affinity in the two assays described above (the direct LTA assay and Fab-capture assay), were further characterized by titration on live whole bacteria. The following four bacterial strains were used: SE4928 (S. epidermidis clinical isolate), SE360 (S. epidermidis clinical isolate), SE1175 (S. epidermidis clinical isolate), and S. epidermidis strain Hay.

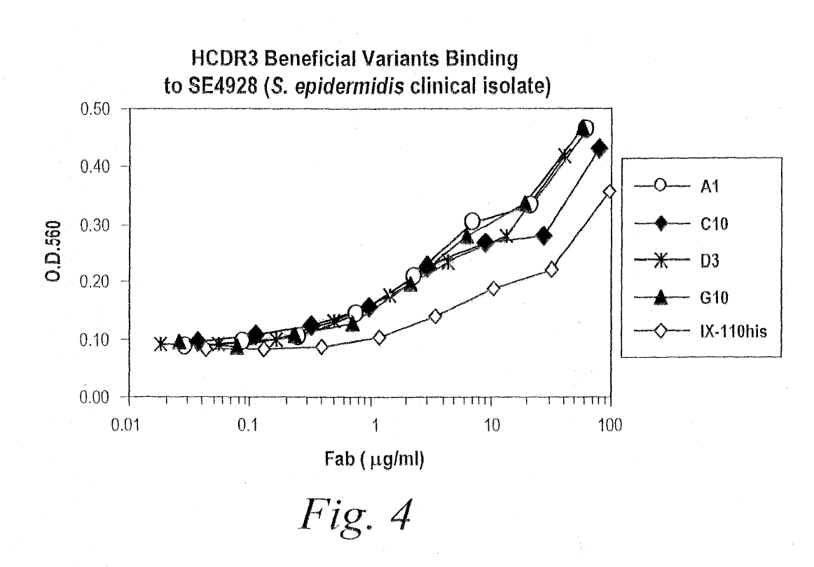

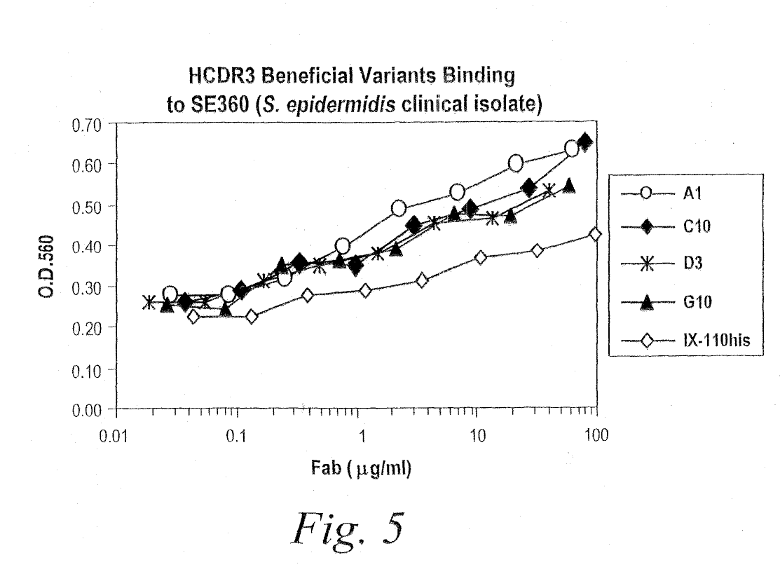

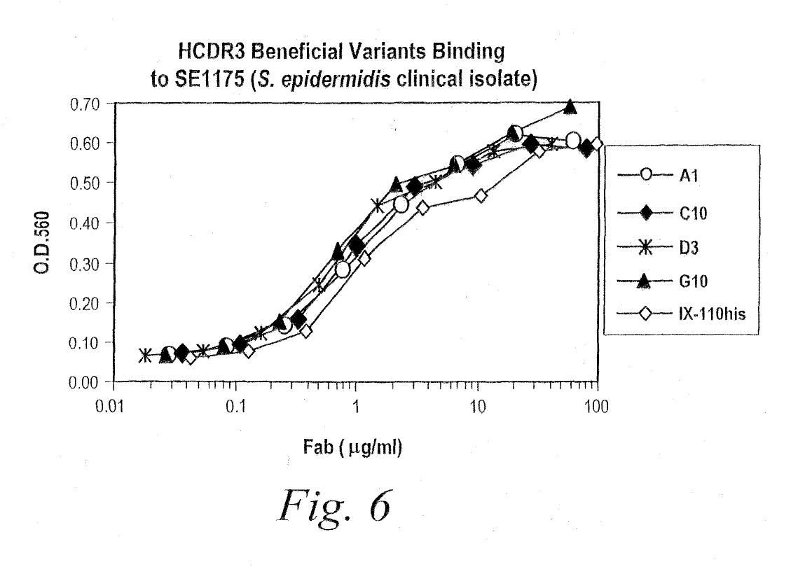

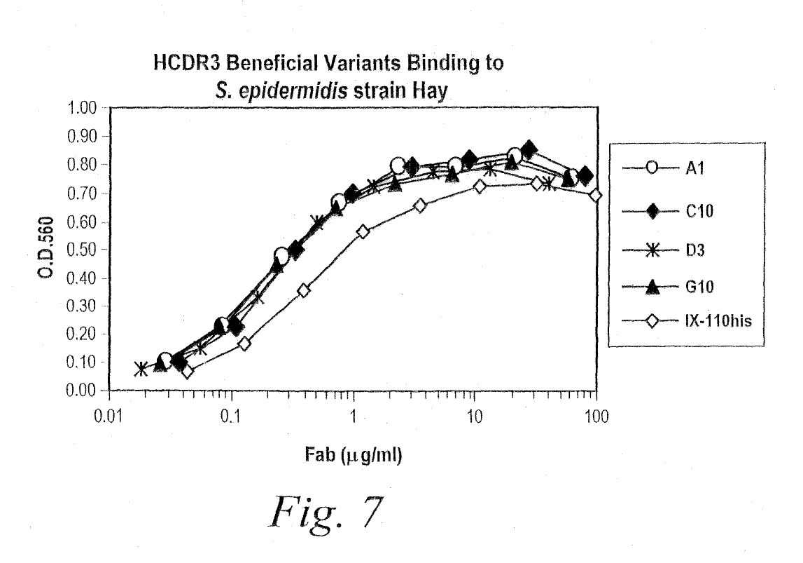

As shown in FIGS. 4-7, all four HCDR3 mutants showed better binding to all four bacterial strains as compared to the parent (IX-110his), e.g., showed better binding to bacterial cells as compared to the parent. Binding was saturated for two highly active strains, S. epidermidis strain Hay and SE1175, at about 2 and 20 μg/ml Fab concentration, respectively. Due to the sticky nature of SE360 cells and consequent difficulty in resuspension, the titrations were less ideal.

### Example 5

### Combinatorial, Variant Library and Affinity Optimized Clones

To engineer a combinatorial variant with further improvement in binding, all single amino acid changes listed in Table 10, which exhibited improved binding when compared to parental A110 by Activity ELISA, were combined to create a combinatorial library. Briefly, a new template with five CDRs deleted (L1, L3, H1, H2 and H3) was created, degenerated primers encoding both parental residue and beneficial mutations in Table 10 were synthesized, and mutagenesis was carried out on the template with a pool of all five primers. The resulting library was characterized by filter lift for Fab expression and by DNA sequencing for mutation distribution, as described supra. The resulting combinatorial library contained 384 different variants.

TABLE 10
Beneficial mutations included in combinatorial
library

| | CDR | | | | | | | |
|---|---|---|---|---|---|---|---|---|
| | L1 | L3 | | H1 | H2 | | H3 | |
| | Amino acid number | | | | | | | |
| | 31* | 92 | 93 | 31 | 52c | 61 | 98 | 100a |
| | Wild type | | | | | | | |
| | N | S | S | N | S | D | S | D |
| Beneficial Mutations | R | R | K | K | K | P | R | H |
| | | | Y | | | | | |

The combinatorial library was screened following the procedure described in Example 2, except using a 10-fold lower antigen concentration in the assays. The selected combinatorial variants are shown in Table 11 below.

TABLE 11
Select combinatorial variants

| | CDR | | | | | | | | | |
|---|---|---|---|---|---|---|---|---|---|---|
| | L1 | L3 | | H1 | H2 | | H3 | | | |
| | Amino acid number | | | | | | | | | |
| | 31* | 92 | 93 | 31 | 52c | 61 | 98 | 100a | | |
| | Wild type | | | | | | | | | |
| | N | S | S | N | S | D | S | D | | |
| | AAT | AGT | AGT | AAC | AGT | GAT | TCA | GAC | | |
| | Beneficial Mutations | | | | | | | | | |
| | | | K/Y | | | | | | LTA- | LBA |
| | R | R | AAG | K | K | P | R | H | bind- | (cell |
| | CGC | AGG | TAT | AAG | AAG | CCG | CGT | CAC | ing | binding) |
| IX110his | | | | | | | | | 1 | 1 |
| H2a-B3 | | | | | K | | | | 3 | 3 |
| H3-A1 | | | | | | | R | | 3 | 3 |
| Com1B12 | R | R | K | | K | | | | 5 | 5 |
| Com2B8 | R | | Y | | | | R | H | 6 | 6 |
| Com1B4 | R | | K | | | | R | | 6 | 7 |
| Com2A8 | | R | K | | K | | | H | 6 | 7 |
| Com1G2 | R | | Y | K | K | | | | 7 | 8 |
| Com2C2 | R | | K | | | | R | H | 8 | 8 |
| Com2C7 | R | | Y | | K | | R | | 8 | 8 |
| Com2H1 | R | | Y | K | K | | | H | 8 | 8 |
| Com2G4 | R | | Y | | K | | R | H | 9 | 8 |
| Com2C5 | R | | K | | K | | | H | 9 | 9 |
| Com2B11 | R | | K | K | | | R | H | 10 | 10 |
| Com2E1 | R | | Y | K | K | | R | | 10 | 10 |

*Numbering according to mouse VK gene alignment (Antibody Group (ABG) web page).
Only mutations are shown. Wild type residues were left as blank.
The numbers in LTA binding and LBA columns are the affinity ranking of the clone. The parental clone is ranked as “1” and the high affinity clones are ranked as “10”. Intermediate numbers indicate an increase in affinity over the parental clone.

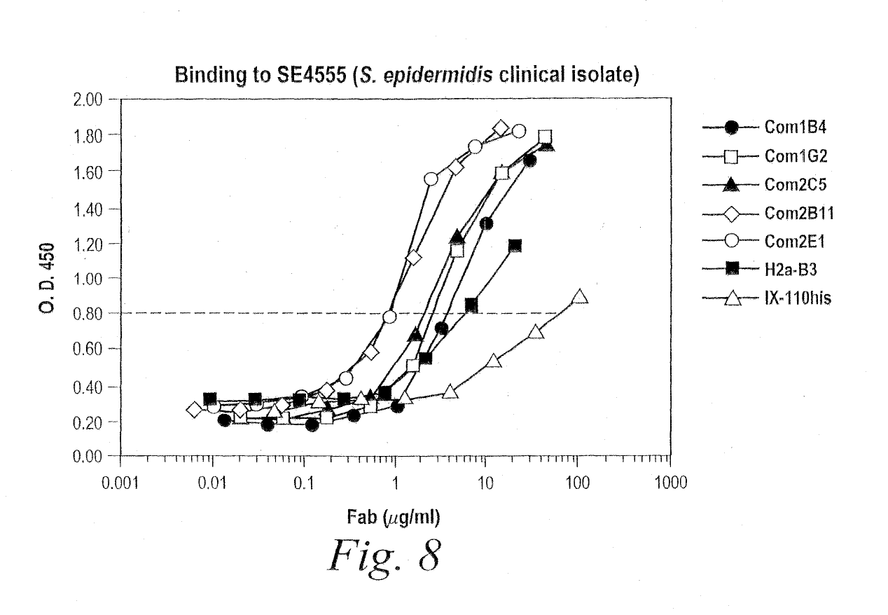

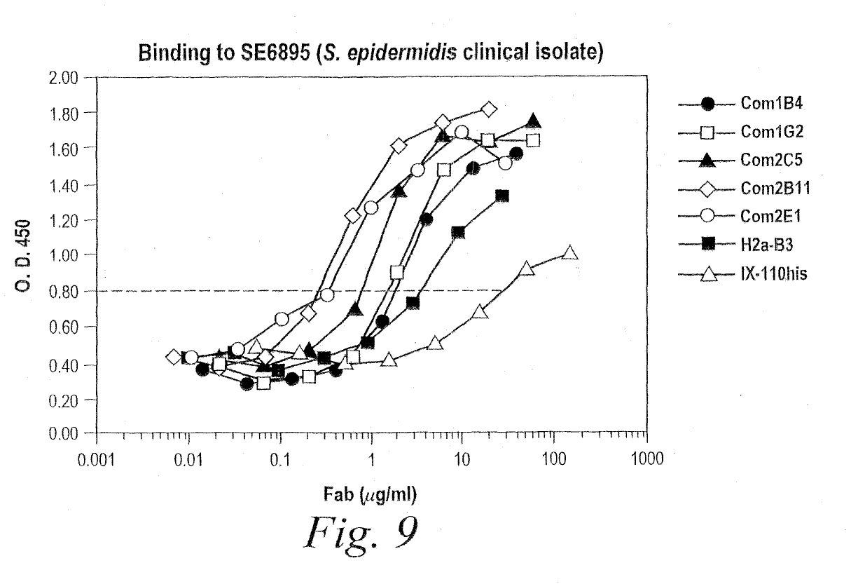

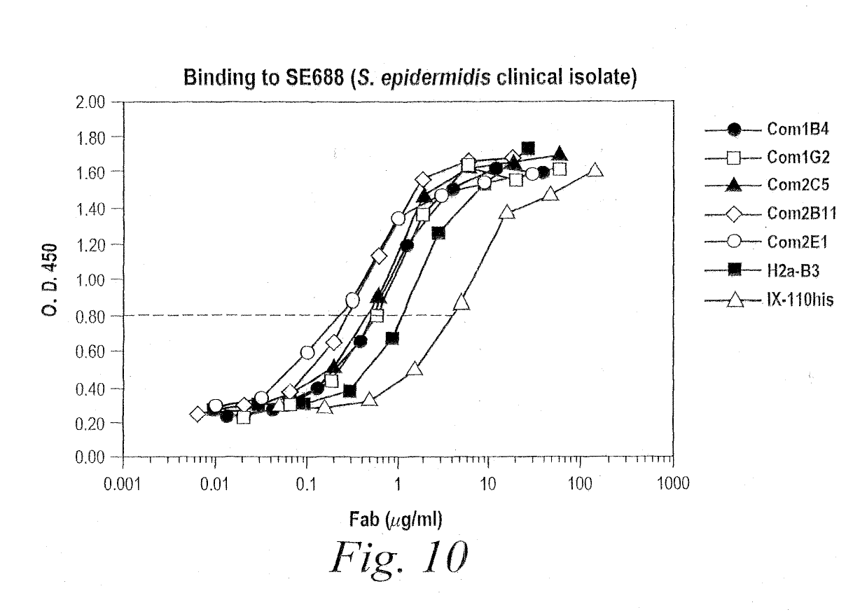

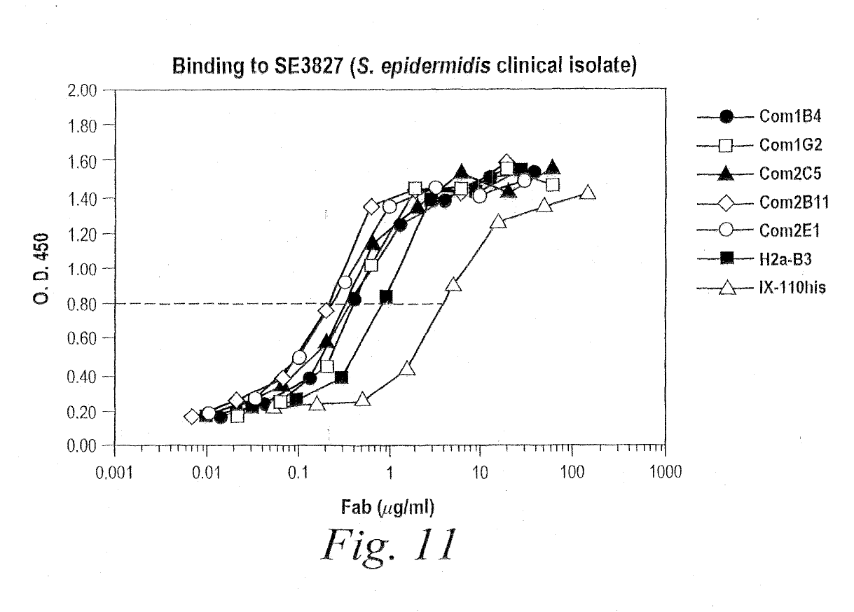

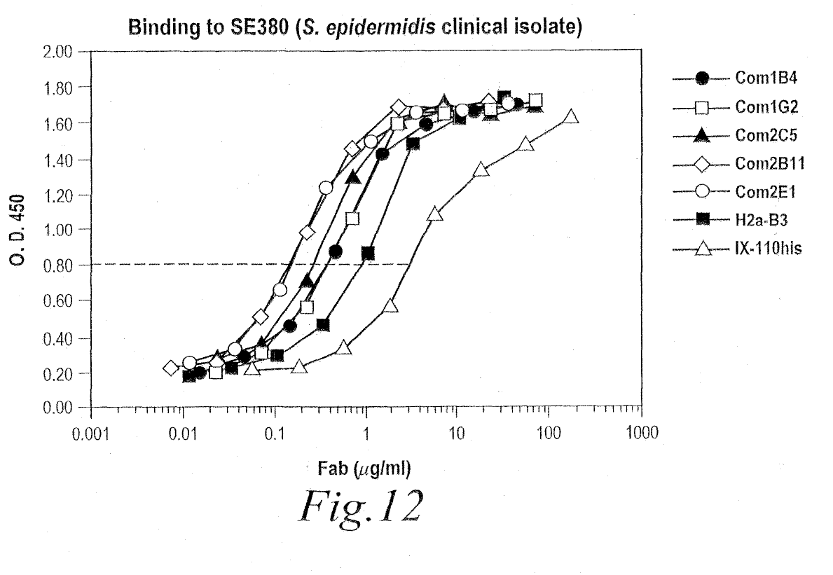

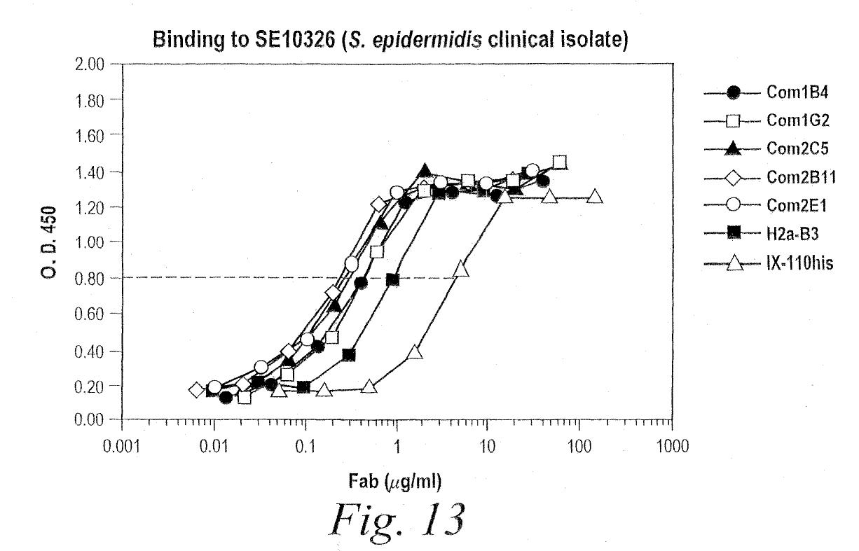

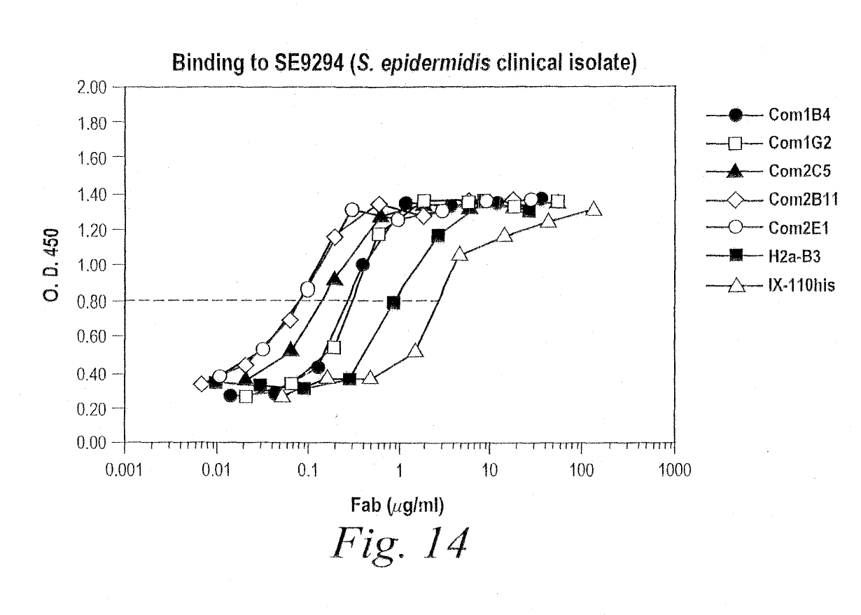

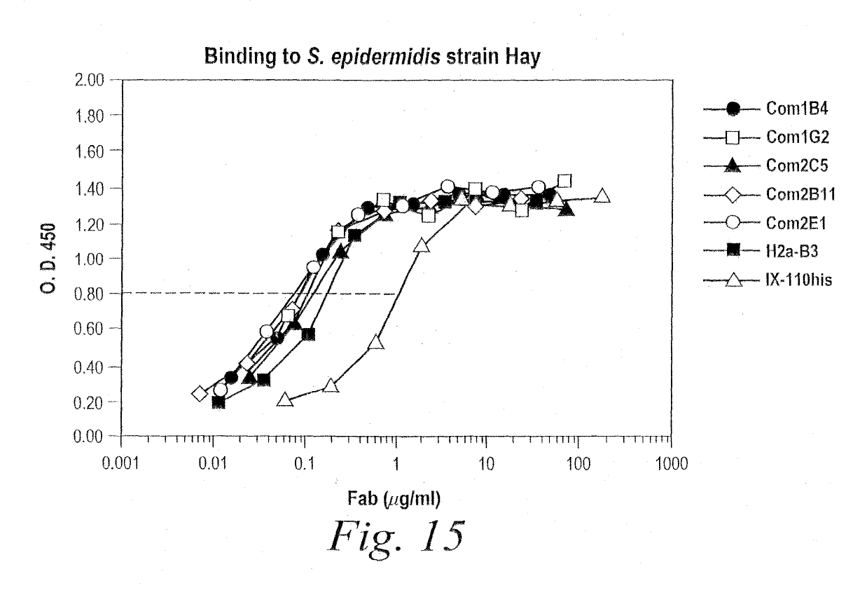

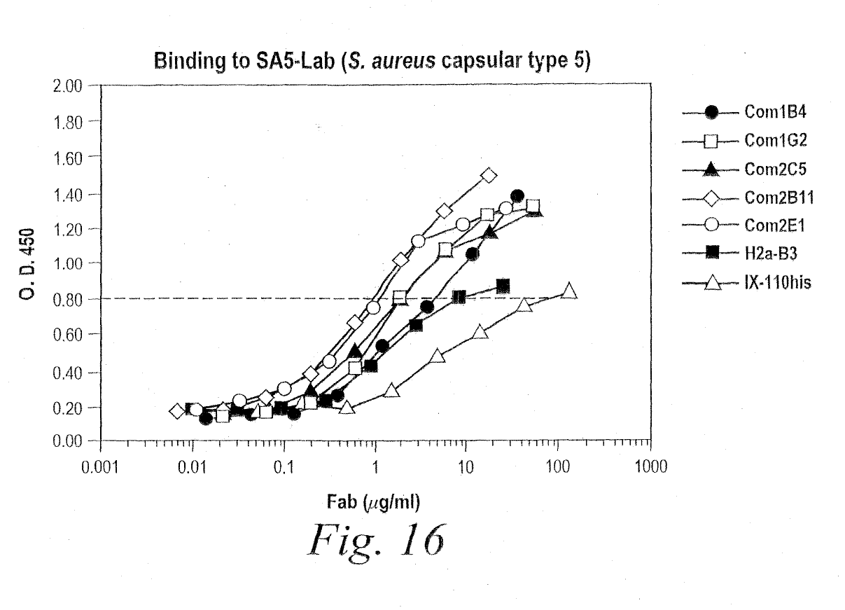

Titration results of combinatorial variants on live bacteria are shown in FIGS. 8-16, which demonstrate that a number of the combinatorial variants exhibit more than a 100-fold increase in binding affinity as compared with the parental A110 antibody. Moreover, the affinity enhancement is widely applicable to all nine different strains assayed.

From the foregoing it will be apparent that the invention provides for a number of uses. For example, the invention provides for the use of any of the antibodies to LTA described above in the treatment, prophylaxis or diagnosis of infections caused by Gram positive bacteria, or in the manufacture of a medicament or diagnostic composition for use in the same.

1. An optimized lipoteichoic acid (LTA) binding molecule, comprising:
a light chain variable region comprising three complementarity determining regions (CDRs), and
a heavy chain variable region comprising three complementarity determining regions (CDRs),
wherein at least one of the light chain variable region or heavy chain variable region comprises at least one modified amino acid residue within the light or heavy chain CDRs, or both, as compared to the CDRs set forth in SEQ ID NO:1 and SEQ ID NO:2, wherein said modified amino acid residue is selected from the group consisting of: 31L, 92L, 93L, 31H, 52cH, 61H, 98H and 100aH, according to Kabat numbering, and combinations thereof,
provided that said binding molecule does not comprise the amino acid sequence set forth as SEQ ID NO:45 or SEQ ID NO:46.

2. The binding molecule of claim 1, wherein the modified amino acid residue is 98H, 100aH or both 98H and 100aH.

3. The binding molecule of claim 2, further comprising a modified amino acid residue at 31L.

4. The binding molecule of claim 2, further comprising a modified amino acid residue at 31H.

5. The binding molecule of claim 3, further comprising a modified amino acid residue at 93L.

6. The binding molecule of claim 2, further comprising a modified amino acid residue at 92L and 52cH.

7. The binding molecule of claim 1, wherein the modified amino acid residues are 31L, 93L and 98H.

8. The binding molecule of claim 1, wherein the modified amino acid residues are 31L, 93L, 98H and 100aH.

9. The binding molecule of claim 1, wherein the modified amino acid residues are 31L, 92L, 93L and 52cH.

10. The binding molecule of claim 1, wherein the modified amino acid residues are 92L, 93L, 52cH and 100aH.

11. The binding molecule of claim 1, wherein the modified amino acid residues are 31L, 93L, 31H and 52cH.

12. The binding molecule of claim 1, wherein the modified amino acid residues are 31L, 93L, 52cH and 98H.

13. The binding molecule of claim 1, wherein the modified amino acid residues are 31L, 93L, 52cH and 100aH.

14. The binding molecule of claim 1, wherein the modified amino acid residues are 31L, 93L, 31H, 52cH and 98H.

15. The binding molecule of claim 1, wherein the modified amino acid residues are 31L, 93L, 31H, 52cH and 100aH.

16. The binding molecule of claim 1, wherein the modified amino acid residues are 31L, 93L, 52cH, 98H and 100aH.

17. The binding molecule of claim 1, wherein the modified amino acid residues are 31L, 93L, 31H, 98H and 100aH.

18. The binding molecule of claim 1, wherein the modified amino acid residue is a positively charged amino acid residue.

19. The binding molecule of claim 1, wherein amino acid residue 31L is Arg.

20. The binding molecule of claim 1, wherein amino acid residue 92L is Arg.

21. The binding molecule of claim 1, wherein amino acid residue 93L is Tyr or Lys.

22. The binding molecule of claim 1, wherein amino acid residue 31H is Lys.

23. The binding molecule of any of claim 1, wherein amino acid residue 52cH is Lys or Arg.

24. The binding molecule of any of claim 1, wherein amino acid residue 98H is Arg or Lys.

25. The binding molecule of claim 1, wherein amino acid residue 100aH is selected from the group consisting of His, Asn, Ala and Arg.

26. The binding molecule of claim 1, wherein amino acid residue 98H is Arg and amino acid residue 100aH is His.

27. The binding molecule of claim 1, wherein amino acid residue 92L is Arg and amino acid residue 93L is Tyr.

28. The binding molecule of claim 1, wherein amino acid residue 92L is Arg and amino acid residue 93L is Lys.

29. The binding molecule of claim 1, wherein the binding molecule has a 5-fold increased binding affinity for LTA as compared to the parent antibody.

30. The binding molecule of claim 1,
wherein at least one amino acid residue within the heavy chain CDR3 and at least one amino acid residue within the light chain CDR1 is modified.

31. The binding molecule of claim 30, wherein the modified amino acid residue within the heavy chain CDR3 is 98H, 100aH, or both 98H and 100aH.

32. The binding molecule of claim 30, wherein the modified amino acid residue within the light chain CDR1 is 31L.

33. The binding molecule of claim 31, wherein the modified amino acid residue 98H is Arg or Lys.

34. The binding molecule of claim 31, wherein the modified amino acid residue 100aH is selected from the group consisting of His, Asn, Ala and Arg.

35. The binding molecule of claim 32, wherein the modified amino acid residue 31L is Arg.

36. The binding molecule of claim 1,
wherein at least one amino acid residue within the heavy chain CDR3 and at least one amino acid residue within the heavy chain CDR1 is modified.

37. The binding molecule of claim 36, wherein the modified amino acid residue within the heavy chain CDR3 is 98H, 100aH, or both 98H and 100aH.

38. The binding molecule of claim 36, wherein the modified amino acid residue within the heavy chain CDR1 is 31H.

39. The binding molecule of claim 37, wherein the modified amino acid residue 98H is Arg or Lys.

40. The binding molecule of claim 37, wherein the modified amino acid residue 100aH is selected from the group consisting of His, Asn, Ala and Arg.

41. The binding molecule of claim 38, wherein the modified amino acid residue 31H is Lys.

42. The binding molecule of claim 1,
wherein at least one amino acid residue within the light chain CDR3, at least one amino acid residue within the heavy chain CDR2, and at least one amino acid residue within the heavy chain CDR3 is modified.

43. The binding molecule of claim 40, wherein the modified amino acid residue within the light chain CDR3 is 93L.

44. The binding molecule of claim 42, wherein the modified amino acid residue within the heavy chain CDR2 is 52cH, 61H, or both 52cH and 61H.

45. The binding molecule of claim 42, wherein the modified amino acid residue within the heavy chain CDR3 is 98H, 100aH, or both 98H and 100aH.

46. The binding molecule of claim 43, wherein the modified amino acid residue 93L is Lys or Tyr.

47. The binding molecule of claim 44, wherein the modified amino acid residue 52cH is Lys or Arg.

48. The binding molecule of claim 44, wherein the modified amino acid residue 61H is Pro.

49. The binding molecule of claim 45, wherein the modified amino acid residue 98H is Arg or Lys.

50. The binding molecule of claim 45, wherein the modified amino acid residue 100aH is selected from the group consisting of His, Asn, Ala and Arg.

51. The binding molecule of claim 1,
wherein at least one amino acid residue within the light chain CDR1, at least one amino acid residue within the light chain CDR3, at least one amino acid residue within the heavy chain CDR2, and at least one amino acid residue within the heavy chain CDR3 is modified.

52. The binding molecule of claim 51, wherein the modified amino acid residue within the light chain CDR1 is 31L.

53. The binding molecule of claim 51, wherein the modified amino acid residue within the light chain CDR3 is 93L.

54. The binding molecule of claim 51, wherein the modified amino acid residue within the heavy chain CDR2 is 52cH, 61H, or both 52cH and 61H.

55. The binding molecule of claim 51, wherein the modified amino acid residue within the heavy chain CDR3 is 98H, 100aH, or both 98H and 100aH.

56. The binding molecule of claim 52, wherein the modified amino acid residue 31L is Arg.

57. The binding molecule of claim 53, wherein the modified amino acid residue 93L is Lys or Tyr.

58. The binding molecule of claim 54, wherein the modified amino acid residue 52cH is Lys or Arg.

59. The binding molecule of claim 54, wherein the modified amino acid residue 61H is Pro.

60. The binding molecule of claim 55, wherein the modified amino acid residue 98H is Arg or Lys.

61. The binding molecule of claim 55, wherein the modified amino acid residue 100aH is selected from the group consisting of His, Asn, Ala and Arg.

62. The binding molecule of claim 1, further comprising at least one additional amino acid residue within the heavy chain CDR3 which is modified as compared to the parent.

63. The binding molecule of claim 62, wherein the at least one additional modified amino acid residue is selected from the group consisting of H54, H99 and H102.

64. The binding molecule of claim 63, wherein the modified amino acid residue H54 is Arg.

65. The binding molecule of claim 63, wherein the modified amino acid residue H99 is Ser or Lys.

66. The binding molecule of claim 63, wherein the modified amino acid residue H102 is Lys.

67. The binding molecule of claim 1, which is a monoclonal antibody or antigen-binding fragment thereof, wherein the antibody comprises a light chain comprising three complementarity determining regions (CDRs) set forth as SEQ ID NO:14, SEQ ID NO:4, and SEQ ID NO:15.

68. The binding molecule of claim 1, which is a monoclonal antibody which or antigen-binding fragment thereof, wherein the antibody comprises a light chain comprising three complementarity determining regions (CDRs) set forth as SEQ ID NO:14, SEQ ID NO:4, and SEQ ID NO:16.

69. The binding molecule of claim 1, which is a monoclonal antibody or antigen-binding fragment thereof, wherein the antibody comprises a light chain comprising three complementarity determining regions (CDRs) set forth as SEQ ID NO:3, SEQ ID NO:4, and SEQ ID NO:17.

70. The binding molecule of claim 1, which is a monoclonal antibody or antigen-binding fragment thereof, wherein the antibody comprises a light chain comprising three complementarity determining regions (CDRs) set forth as SEQ ID NO:14, SEQ ID NO:4, and SEQ ID NO:17.

71. The binding molecule of claim 1, which is a monoclonal antibody or antigen-binding fragment thereof, wherein the antibody comprises a light chain comprising three complementarity determining regions (CDRs) set forth as SEQ ID NO:3, SEQ ID NO:4, and SEQ ID NO:18.

72. The binding molecule of claim 1, which is a monoclonal antibody or antigen-binding fragment thereof, wherein the antibody comprises a light chain comprising three complementarity determining regions (CDRs) set forth as SEQ ID NO:14, SEQ ID NO:4, and SEQ ID NO:5.

73. The binding molecule of claim 1, which is a monoclonal antibody or antigen-binding fragment thereof, wherein the antibody comprises a heavy chain comprising three complementarity determining regions (CDRs) set forth as SEQ ID NO:11, SEQ ID NO:7, and SEQ ID NO:19.

74. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:7, and SEQ ID NO:19.

75. The binding molecule of claim 1, which is a monoclonal antibody or antigen-binding fragment thereof, wherein the antibody comprises a heavy chain comprising three complementarity determining regions (CDRs) set forth as SEQ ID NO:20, SEQ ID NO:21, and SEQ ID NO:8.

76. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:21, and SEQ ID NO:22.

77. The binding molecule of claim 1, which is a monoclonal antibody or antigen-binding fragment thereof, wherein the antibody comprises a heavy chain comprising three complementarity determining regions (CDRs) set forth as SEQ ID NO:20, SEQ ID NO:21, and SEQ ID NO:23.

78. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:21, and SEQ ID NO:19.

79. The binding molecule of claim 1, which is a monoclonal antibody or antigen-binding fragment thereof, wherein the antibody comprises a heavy chain comprising three complementarity determining regions (CDRs) set forth as SEQ ID NO:20, SEQ ID NO:21, and SEQ ID NO:22.

80. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:7, and SEQ ID NO:22.

81. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:21, and SEQ ID NO:23.

82. The binding molecule of claim 1, which is a monoclonal antibody or antigen-binding fragment thereof, wherein the antibody comprises a heavy chain comprising three complementarity determining regions (CDRs) set forth as SEQ ID NO:20, SEQ ID NO:7, and SEQ ID NO:19.

83. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:21, and SEQ ID NO:8.

84. The binding molecule of claim 63, which is a monoclonal antibody or antigen-binding fragment thereof, wherein the antibody comprises a heavy chain comprising three complementarity determining regions (CDRs) set forth as SEQ ID NO:6, SEQ ID NO:24, and SEQ ID NO:22.

85. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:7, and SEQ ID NO:25.

86. The binding molecule of claim 63, which is a monoclonal antibody or antigen-binding fragment thereof, wherein the antibody comprises a heavy chain comprising three complementarity determining regions (CDRs) set forth as SEQ ID NO:6, SEQ ID NO:7, and SEQ ID NO:26.

87. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:27, and SEQ ID NO:23.

88. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:7, and SEQ ID NO:28.

89. The binding molecule of claim 63, which is a monoclonal antibody or antigen-binding fragment thereof, wherein the antibody comprises a heavy chain comprising three complementarity determining regions (CDRs) set forth as SEQ ID NO:20, SEQ ID NO:7, and SEQ ID NO:29.

90. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:7, and SEQ ID NO:30.

91. The binding molecule of claim 1, which is a monoclonal antibody or antigen-binding fragment thereof, wherein the antibody comprises a heavy chain comprising three complementarity determining regions (CDRs) set forth as SEQ ID NO:6, SEQ ID NO:7, and SEQ ID NO:31.

92. The binding molecule of claim 63, which is a monoclonal antibody or antigen-binding fragment thereof, wherein the antibody comprises a heavy chain comprising three complementarity determining regions (CDRs) set forth as SEQ ID NO:6, SEQ ID NO:7, and SEQ ID NO:32.

93. The binding molecule of claim 1, wherein the binding molecule, monoclonal antibody or antigen binding fragment thereof specifically binds whole bacteria.

94. The binding molecule, of claim 1, wherein said binding molecule is selected from the group consisting of: a whole antibody, an antibody fragment, a humanized antibody, a human antibody, a single chain antibody, an immunoconjugate, a defucosylated antibody, an aglycosylated antibody, and a bispecific antibody.

95. The binding molecule of claim 94, wherein the antibody fragment is selected from the group consisting of a Fab fragment, a Fab′ fragment, a F(ab)₂ fragment, and a Fᵥ fragment.

96. A cell producing the binding molecule of claim 1.

97. A composition comprising a binding molecule of claim 1 and a pharmaceutically acceptable carrier.

98. A method of preventing a Staphylococcal infection in a human comprising administering the composition of claim 97 to the human.

99. An isolated nucleic acid molecule comprising-a nucleic acid selected from the group consisting of SEQ ID NO:108, 109, 110, 111, 112, 113, 114 and 115.

100. An expression vector comprising the nucleic acid of claim 99.

101. A cell comprising the expression vector of claim 100.

102. An isolated peptide, wherein the peptide comprises an amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31 and SEQ ID NO:32.
